SEQUENCE LISTING (115 sequences)

SEQ ID NO: 1 - DNA, 1522 bp, HUMAN
  CDS (13)..(1389)
  mat_peptide (124)..(1389)
acagtcagccgcatggctcccctgtgccccagcccctggctccctctgttgatcccggcccctgctccaggcctcactgtgcaactgctgctgtcactgctgcttctgatgcctgtccatccccagaggttgccccggatgcaggaggattcccccttgggaggaggctcttctggggaagatgacccactgggcgaggaggatctgcccagtgaagaggattcacccagagaggaggatccacccggagaggaggatctacctggagaggaggatctacctggagaggaggatctacctgaagttaagcctaaatcagaagaagagggctccctgaagttagaggatctacctactgttgaggctcctggagatcctcaagaaccccagaataatgcccacagggacaaagaaggggatgaccagagtcattggcgctatggaggcgacccgccctggccccgggtgtccccagcctgcgcgggccgcttccagtccccggtggatatccgcccccagctcgccgccttctgcccggccctgcgccccctggaactcctgggcttccagctcccgccgctcccagaactgcgcctgcgcaacaatggccacagtgtgcaactgaccctgcctcctgggctagagatggctctgggtcccgggcgggagtaccgggctctgcagctgcatctgcactggggggctgcaggtcgtccgggctcggagcacactgtggaaggccaccgtttccctgccgagatccacgtggttcacctcagcaccgcctttgccagagttgacgaggccttggggcgcccgggaggcctggccgtgttggccgcctttctggaggagggcccggaagaaaacagtgcctatgagcagttgctgtctcgcttggaagaaatcgctgaggaaggctcagagactcaggtcccaggactggacatatctgcactcctgccctctgacttcagccgctacttccaatatgaggggtctctgactacaccgccctgtgcccagggtgtcatctggactgtgtttaaccagacagtgatgctgagtgctaagcagctccacaccctctctgacaccctgtggggacctggtgactctcggctacagctgaacttccgagcgacgcagcctttgaatgggcgagtgattgaggcctccttccctgctggagtggacagcagtcctcgggctgctgagccagtccagctgaattcctgcctggctgctggtgacatcctagccctggtttttggcctcctttttgctgtcaccagcgtcgcgttccttgtgcagatgagaaggcagcacagaaggggaaccaaagggggtgtgagctaccgcccagcagaggtagccgagactggagcctagaggctggatcttggagaatgtgagaagccagccagaggcatctgagggggagccggtaactgtcctgtcctgctcattatgccacttccttttaactgccaagaaattttttaaaataaatatttataat

SEQ ID NO: 2 - protein, 459 aa, HUMAN
MAPLCPSPWLPLLIPAPAPGLTVQLLLSLLLLMPVHPQRLPRMQEDSPLGGGSSGEDDPLGEEDLPSEEDSPREEDPPGEEDLPGEEDLPGEEDLPEVKPKSEEEGSLKLEDLPTVEAPGDPQEPQNNAHRDKEGDDQSHWRYGGDPPWPRVSPACAGRFQSPVDIRPQLAAFCPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFPAGVDSSPRAAEPVQLNSCLAAGDILALVFGLLFAVTSVAFLVQMRRQHRRGTKGGVSYRPAEVAETGA

SEQ ID NO: 3 - DNA, 29 bp, HUMAN
cgcccagtgggtcatcttccccagaagag

SEQ ID NO: 4 - DNA, 19 bp, HUMAN
ggaatcctcctgcatccgg

SEQ ID NO: 5 - DNA, 10898 bp, HUMAN
  gene (1)..(10898): full-length MN genomic sequence
  unsure of base at position 1974 (1974): unsure of base at position 1974, which is in the 5′ region flanking the transcription initiation site (3507) as determined by RNase protection assay.
ggatcctgttgactcgtgaccttacccccaaccctgtgctctctgaaacatgagctgtgtccactcagggttaaatggattaagggcggtgcaagatgtgctttgttaaacagatgcttgaaggcagcatgctcgttaagagtcatcaccaatccctaatctcaagtaatcagggacacaaacactgcggaaggccgcagggtcctctgcctaggaaaaccagagacctttgttcacttgtttatctgaccttccctccactattgtccatgaccctgccaaatccccctctgtgagaaacacccaagaattatcaataaaaaaataaatttaaaaaaaaaatacaaaaaaaaaaaaaaaaaaaaaaaaagacttacgaatagttattgataaatgaatagctattggtaaagccaagtaaatgatcatattcaaaaccagacggccatcatcacagctcaagtctacctgatttgatctctttatcattgtcattctttggattcactagattagtcatcatcctcaaaattctcccccaagttctaattacgttccaaacatttaggggttacatgaagcttgaacctactaccttctttgcttttgagccatgagttgtaggaatgatgagtttacaccttacatgctggggattaatttaaactttacctctaagtcagttgggtagcctttggcttatttttgtagctaattttgtagttaatggatgcactgtgaatcttgctatgatagttttcctccacactttgccactaggggtaggtaggtactcagttttcagtaattgcttacctaagaccctaagccctatttctcttgtactggcctttatctgtaatatgggcatatttaatacaatataatttttggagtttttttgtttgtttgtttgtttgtttttttgagacggagtcttgcatctgtcatgcccaggctggagtagcagtggtgccatctcggctcactgcaagctccacctcccgagttcacgccattttcctgcctcagcctcccgagtagctgggactacaggcgcccgccaccatgcccggctaattttttgtatttttggtagagacggggtttcaccgtgttagccagaatggtctcgatctcctgacttcgtgatccacccgcctcggcctcccaaagttctgggattacaggtgtgagccaccgcacctggccaattttttgagtcttttaaagtaaaaatatgtcttgtaagctggtaactatggtacatttccttttattaatgtggtgctgacggtcatataggttcttttgagtttggcatgcatatgctactttttgcagtcctttcattacatttttctctcttcatttgaagagcatgttatatcttttagcttcacttggcttaaaaggttctctcattagcctaacacagtgtcattgttggtaccacttggatcataagtggaaaaacagtcaagaaattgcacagtaatacttgtttgtaagagggatgattcaggtgaatctgacactaagaaactcccctacctgaggtctgagattcctctgacattgctgtatataggcttttcctttgacagcctgtgactgcggactatttttcttaagcaagatatgctaaagttttgtgagcctttttccagagagaggtctcatatctgcatcaagtgagaacatataatgtctgcatgtttccatatttcaggaatgtttgcttgtgttttatgcttttatatagacagggaaacttgttcctcagtgacccaaaagaggtgggaattgttattggatatcatcattggcccacgctttctgaccttggaaacaattaagggttcataatctcaattctgtcagaattggtacaagaaatagctgctatgtttcttgacattccacttggtaggaaataagaatgtgaaactcttcagttggtgtgtgtccctngtttttttgcaatttccttcttactgtgttaaaaaaaagtatgatcttgctctgagaggtgaggcattcttaatcatgatctttaaagatcaataatataatcctttcaaggattatgtctttattataataaagataatttgtctttaacagaatcaataatataatcccttaaaggattatatctttgctgggcgcagtggctcacacctgtaatcccagcactttgggtggccaaggtggaaggatcaaatttgcctacttctatattatcttctaaagcagaattcatctctcttccctcaatatgatgatattgacagggtttgccctcactcactagattgtgagctcctgctcagggcaggtagcgttttttgtttttgtttttgtttttcttttttgagacagggtcttgctctgtcacccaggccagagtgcaatggtacagtctcagctcactgcagcctcaaccgcctcggctcaaaccatcatcccatttcagcctcctgagtagctgggactacaggcacatgccattacacctggctaatttttttgtatttctagtagagacagggtttggccatgttgcccgggctggtctcgaactcctggactcaagcaatccacccacctcagcctcccaaaatgagggaccgtgtcttattcatttccatgtccctagtccatagcccagtgctggacctatggtagtactaaataaatatttgttgaatgcaatagtaaatagcatttcagggagcaagaactagattaacaaaggtggtaaaaggtttggagaaaaaaataatagtttaatttggctagagtatgagggagagtagtaggagacaagatggaaaggtctcttgggcaaggttttgaaggaagttggaagtcagaagtacacaatgtgcatatcgtggcaggcagtggggagccaatgaaggcttttgagcaggagagtaatgtgttgaaaaataaatataggttaaacctatcagagcccctctgacacatacacttgcttttcattcaagctcaagtttgtctcccacatacccattacttaactcaccctcgggctcccctagcagcctgccctacctctttacctgcttcctggtggagtcagggatgtatacatgagctgctttccctctcagccagaggacatggggggccccagctcccctgcctttccccttctgtgcctggagctgggaagcaggccagggttagctgaggctggctggcaagcagctgggtggtgccagggagagcctgcatagtgccaggtggtgccttgggttccaagctagtccatggccccgataaccttctgcctgtgcacacacctgcccctcactccacccccatcctagctttggtatgggggagagggcacagggccagacaaacctgtgagactttggctccatctctgcaaaagggcgctctgtgagtcagcctgctcccctccaggcttgctcctcccccacccagctctcgtttccaatgcacgtacagcccgtacacaccgtgtgctgggacaccccacagtcagccgcatggctcccctgtgccccagcccctggctccctctgttgatcccggcccctgctccaggcctcactgtgcaactgctgctgtcactgctgcttctggtgcctgtccatccccagaggttgccccggatgcaggaggattcccccttgggaggaggctcttctggggaagatgacccactgggcgaggaggatctgcccagtgaagaggattcacccagagaggaggatccacccggagaggaggatctacctggagaggaggatctacctggagaggaggatctacctgaagttaagcctaaatcagaagaagagggctccctgaagttagaggatctacctactgttgaggctcctggagatcctcaagaaccccagaataatgcccacagggacaaagaaggtaagtggtcatcaatctccaaatccaggttccaggaggttcatgactcccctcccataccccagcctaggctctgttcactcagggaaggaggggagactgtactccccacagaagcccttccagaggtcccataccaatatccccatccccactctcggaggtagaaagggacagatgtggagagaaaataaaaagggtgcaaaaggagagaggtgagctggatgagatgggagagaagggggaggctggagaagagaaagggatgagaactgcagatgagagaaaaaatgtgcagacagaggaaaaaaataggtggagaaggagagtcagagagtttgaggggaagagaaaaggaaagcttgggaggtgaagtgggtaccagagacaagcaagaagagctggtagaagtcatctcatcttaggctacaatgaggaattgagacctaggaagaagggacacagcaggtagagaaacgtggcttcttgactcccaagccaggaatttggggaaaggggttggagaccatacaaggcagagggatgagtggggagaagaaagaagggagaaaggaaagatggtgtactcactcatttgggactcaggactgaagtgcccactcactttttttttttttttttttgagacaaactttcacttttgttgcccaggctggagtgcaatggcgcgatctcggctcactgcaacctccacctcccgggttcaagtgattctcctgcctcagcctctagccaagtagctgcgattacaggcatgcgccaccacgcccggctaatttttgtatttttagtagagacggggtttcgccatgttggtcaggctggtctcgaactcctgatctcaggtgatccaaccaccctggcctcccaaagtgctgggattataggcgtgagccacagcgcctggcctgaagcagccactcacttttacagaccctaagacaatgattgcaagctggtaggattgctgtttggcccacccagctgcggtgttgagtttgggtgcggtctcctgtgctttgcacctggcccgcttaaggcatttgttacccgtaatgctcctgtaaggcatctgcgtttgtgacatcgttttggtcgccaggaagggattggggctctaagcttgagcggttcatccttttcatttatacaggggatgaccagagtcattggcgctatggaggtgagacacccacccgctgcacagacccaatctgggaacccagctctgtggatctcccctacagccgtccctgaacactggtcccgggcgtcccacccgccgcccaccgtcccaccccctcaccttttctacccgggttccctaagttcctgacctaggcgtcagacttcctcactatactctcccaccccaggcgacccgccctggccccgggtgtccccagcctgcgcgggccgcttccagtccccggtggatatccgcccccagctcgccgccttctgcccggccctgcgccccctggaactcctgggcttccagctcccgccgctcccagaactgcgcctgcgcaacaatggccacagtggtgagggggtctccccgccgagacttggggatggggcggggcgcagggaagggaaccgtcgcgcagtgcctgcccgggggttgggctggccctaccgggcggggccggctcacttgcctctccctacgcagtgcaactgaccctgcctcctgggctagagatggctctgggtcccgggcgggagtaccgggctctgcagctgcatctgcactggggggctgcaggtcgtccgggctcggagcacactgtggaaggccaccgtttccctgccgaggtgagcgcggactggccgagaaggggcaaaggagcggggcggacgggggccagagacgtggccctctcctaccctcgtgtccttttcagatccacgtggttcacctcagcaccgcctttgccagagttgacgaggccttggggcgcccgggaggcctggccgtgttggccgcctttctggaggtaccagatcctggacaccccctactccccgctttcccatcccatgctcctcccggactctatcgtggagccagagaccccatcccagcaagctcactcaggcccctggctgacaaactcattcacgcactgtttgttcatttaacacccactgtgaaccaggcaccagcccccaacaaggattctgaagctgtaggtccttgcctctaaggagcccacagccagtgggggaggctgacatgacagacacataggaaggacatagtaaagatggtggtcacagaggaggtgacacttaaagccttcactggtagaaaagaaaaggaggtgttcattgcagaggaaacagaatgtgcaaagactcagaatatggcctatttagggaatggctacatacaccatgattagaggaggcccagtaaagggaagggatggtgagatgcctgctaggttcactcactcacttttatttatttatttatttttttgacagtctctctgtcgcccaggctggagtgcagtggtgtgatcttgggtcactgcaacttccgcctcccgggttcaagggattctcctgcctcagcttcctgagtagctggggttacaggtgtgtgccaccatgcccagctaatttttttttgtatttttagtagacagggtttcaccatgttggtcaggctggtctcaaactcctggcctcaagtgatccgcctgactcagcctaccaaagtgctgattacaagtgtgagccaccgtgcccagccacactcactgattctttaatgccagccacacagcacaaagttcagagaaatgcctccatcatagcatgtcaatatgttcatactcttaggttcatgatgttcttaacattaggttcataagcaaaataagaaaaaagaataataaataaaagaagtggcatgtcaggacctcacctgaaaagccaaacacagaatcatgaaggtgaatgcagaggtgacaccaacacaaaggtgtatatatggtttcctgtggggagtatgtacggaggcagcagtgagtgagactgcaaacgtcagaagggcacgggtcactgagagcctagtatcctagtaaagtgggctctctccctctctctccagcttgtcattgaaaaccagtccaccaagcttgttggttcgcacagcaagagtacatagagtttgaaataatacataggattttaagagggagacactgtctctaaaaaaaaaaacaacagcaacaacaaaaagcaacaaccattacaattttatgttccctcagcattctcagagctgaggaatgggagaggactatgggaacccccttcatgttccggccttcagccatggccctggatacatgcactcatctgtcttacaatgtcattcccccaggagggcccggaagaaaacagtgcctatgagcagttgctgtctcgcttggaagaaatcgctgaggaaggtcagtttgttggtctggccactaatctctgtggcctagttcataaagaatcaccctttggagcttcaggtctgaggctggagatgggctccctccagtgcaggagggattgaagcatgagccagcgctcatcttgataataaccatgaagctgacagacacagttacccgcaaacggctgcctacagattgaaaaccaagcaaaaaccgccgggcacggtggctcacgcctgtaatcccagcactttgggaggccaaggcaggtggatcacgaggtcaagagatcaagaccatcctggccaacatggtgaaaccccatctctactaaaaatacgaaaaaatagccaggcgtggtggcgggtgcctgtaatcccagctactcgggaggctgaggcaggagaatggcatgaacccgggaggcagaagttgcagtgagccgagatcgtgccactgcactccagcctgggcaacagagcgagactcttgtctcaaaaaaaaaaaaaaaaaagaaaaccaagcaaaaaccaaaatgagacaaaaaaaacaagaccaaaaaatggtgtttggaaattgtcaaggtcaagtctggagagctaaactttttctgagaactgtttatctttaataagcatcaaatattttaactttgtaaatacttttgttggaaatcgttctcttcttagtcactcttgggtcattttaaatctcacttactctactagaccttttaggtttctgctagactaggtagaactctgcctttgcatttcttgtgtctgttttgtatagttatcaatattcatatttatttacaagttattcagatcattttttcttttcttttttttttttttttttttttttacatctttagtagagacagggtttcaccatattggccaggctgctctcaaactcctgaccttgtgatccaccagcctcggcctcccaaagtgctgggattcattttttctttttaatttgctctgggcttaaacttgtggcccagcactttatgatggtacacagagttaagagtgtagactcagacggtctttcttctttccttctcttccttcctcccttccctcccaccttcccttctctccttcctttctttcttcctctcttgcttcctcaggcctcttccagttgctccaaagccctgtacttttttttgagttaacgtcttatgggaagggcctgcacttagtgaagaagtggtctcagagttgagttaccttggcttctgggaggtgaaactgtatccctataccctgaagctttaagggggtgcaatgtagatgagaccccaacatagatcctcttcacaggctcagagactcaggtcccaggactggacatatctgcactcctgccctctgacttcagccgctacttccaatatgaggggtctctgactacaccgccctgtgcccagggtgtcatctggactgtgtttaaccagacagtgatgctgagtgctaagcaggtgggcctggggtgtgtgtggacacagtgggtgcgggggaaagaggatgtaagatgagatgagaaacaggagaagaaagaaatcaaggctgggctctgtggcttacgcctataatcccaccacgttgggaggctgaggtgggagaatggtttgagcccaggagttcaagacaaggcggggcaacatagtgtgaccccatctctaccaaaaaaaccccaacaaaaccaaaaatagccgggcatggtggtatgcggcctagtcccagctactcaaggaggctgaggtgggaagatcgcttgattccaggagtttgagactgcagtgagctatgatcccaccactgcctaccatctttaggatacatttatttatttataaaagaaatcaagaggctggatggggaatacaggagctggagggtggagccctgaggtgctggttgtgagctggcctgggacccttgtttcctgtcatgccatgaacccacccacactgtccactgacctccctagctccacaccctctctgacaccctgtggggacctggtgactctcggctacagctgaacttccgagcgacgcagcctttgaatgggcgagtgattgaggcctccttccctgctggagtggacagcagtcctcgggctgctgagccaggtacagctttgtctggtttccccccagccagtagtcccttatcctcccatgtgtgtgccagtgtctgtcattggtggtcacagcccgcctctcacatctcctttttctctccagtccagctgaattcctgcctggctgctggtgagtctgcccctcctcttggtcctgatgccaggagactcctcagcaccattcagccccagggctgctcaggaccgcctctgctccctctccttttctgcagaacagaccccaaccccaatattagagaggcagatcatggtggggattcccccattgtccccagaggctaattgattagaatgaagcttgagaaatctcccagcatccctctcgcaaaagaatccccccccctttttttaaagatagggtctcactctgtttgccccaggctggggtgttgtggcacgatcatagctcactgcagcctcgaactcctaggctcaggcaatcctttcaccttagcttctcaaagcactgggactgtaggcatgagccactgtgcctggccccaaacggcccttttacttggcttttaggaagcaaaaacggtgcttatcttaccccttctcgtgtatccaccctcatcccttggctggcctcttctggagactgaggcactatggggctgcctgagaactcggggcaggggtggtggagtgcactgaggcaggtgttgaggaactctgcagacccctcttccttcccaaagcagccctctctgctctccatcgcaggtgacatcctagccctggtttttggcctcctttttgctgtcaccagcgtcgcgttccttgtgcagatgagaaggcagcacaggtattacactgaccctttcttcaggcacaagcttcccccacccttgtggagtcacttcatgcaaagcgcatgcaaatgagctgctcctgggccagttttctgattagcctttcctgttgtgtacacacagaaggggaaccaaagggggtgtgagctaccgcccagcagaggtagccgagactggagcctagaggctggatcttggagaatgtgagaagccagccagaggcatctgagggggagccggtaactgtcctgtcctgctcattatgccacttccttttaactgccaagaaattttttaaaataaatatttataataaaatatgtgttagtcacctttgttccccaaatcagaaggaggtatttgaatttcctattactgttattagcaccaatttagtggtaatgcatttattctattacagttcggcctccttccacacatcactccaatgtgttgctcc

SEQ ID NO: 6 - protein, 37 aa, HUMAN
MAPLCPSPWLPLLIPAPAPGLTVQLLLSLLLLMPVHP

SEQ ID NO: 7 - DNA, 25 bp, HUMAN
tggggttcttgaggatctccaggag

SEQ ID NO: 8 - DNA, 26 bp, HUMAN
ctctaacttcagggagccctcttctt

SEQ ID NO: 9 - DNA, 48 bp, HUMAN
  primer_bind (1)..(48): anchor primer that anneals to the homopolymeric tail.
  inosine (36)..(37) (41)..(42) (46)..(47): each of the modified_bases at positions (36), (37), (41), (42), (46) and (47) are inosine
cuacuacuacuaggccacgcgtcgactagtacgggaagggaagggaag

SEQ ID NO: 10 - protein, 6 aa, HUMAN
EEDLPS

SEQ ID NO: 11 - protein, 6 aa, HUMAN
GEDDPL

SEQ ID NO: 12 - protein, 21 aa, HUMAN
NNAHRDKEGDDQSHWRYGGDP

SEQ ID NO: 13 - protein, 16 aa, HUMAN
HPQRLPRMQEDSPLGG

SEQ ID NO: 14 - protein, 24 aa, HUMAN
EEDSPREEDPPGEEDLPGEEDLPG

SEQ ID NO: 15 - protein, 13 aa, HUMAN
LEEGPEENSAYEQ

SEQ ID NO: 16 - protein, 16 aa, HUMAN
MRRQHRRGTKGGVSYR

SEQ ID NO: 17 - DNA, 10 bp, HUMAN
ctccatctct

SEQ ID NO: 18 - DNA, 10 bp, HUMAN
ccacccccat

SEQ ID NO: 19 - DNA, 205 bp, HUMAN
acctgcccctcactccacccccatcctagctttggtatgggggagagggcacagggccagacaaacctgtgagactttggctccatctctgcaaaagggcgctctgtgagtcagcctgctcccctccaggcttgctcctcccccacccagctctcgtttccaatgcacgtacagcccgtacacaccgtgtgctgggacaccccac

SEQ ID NO: 20 - DNA, 10 bp, HUMAN
  misc_feature (1)..(10)
yyycayyyyy

SEQ ID NO: 21 - DNA, 10 bp, HUMAN
  ref <301>: AUTHORS: Locker and Buzard,
  ref <303>: JOURNAL: DNA Sequencing and Mapping
  ref <304>: VOLUME: 1
  ref <306>: PAGES: 3-11
  ref <307>: DATE: 1990
tgtgagactt

SEQ ID NO: 22 - DNA, 540 bp, HUMAN
  promoter (1)..(540)
cttgcttttcattcaagctcaagtttgtctcccacatacccattacttaactcaccctcgggctcccctagcagcctgccctacctctttacctgcttcctggtggagtcagggatgtatacatgagctgctttccctctcagccagaggacatggggggccccagctcccctgcctttccccttctgtgcctggagctgggaagcaggccagggttagctgaggctggctggcaagcagctgggtggtgccagggagagcctgcatagtgccaggtggtgccttgggttccaagctagtccatggccccgataaccttctgcctgtgcacacacctgcccctcactccacccccatcctagctttggtatgggggagagggcacagggccagacaaacctgtgagactttggctccatctctgcaaaagggcgctctgtgagtcagcctgctcccctccaggcttgctcctcccccacccagctctcgtttccaatgcacgtacagcccgtacacaccgtgtgctgggacaccccacag

SEQ ID NO: 23 - DNA, 445 bp, HUMAN
  exon (1): 1st MN exon
gcccgtacacaccgtgtgctgggacaccccacagtcagccgcatggctcccctgtgccccagcccctggctccctctgttgatcccggcccctgctccaggcctcactgtgcaactgctgctgtcactgctgcttctggtgcctgtccatccccagaggttgccccggatgcaggaggattcccccttgggaggaggctcttctggggaagatgacccactgggcgaggaggatctgcccagtgaagaggattcacccagagaggaggatccacccggagaggaggatctacctggagaggaggatctacctggagaggaggatctacctgaagttaagcctaaatcagaagaagagggctccctgaagttagaggatctacctactgttgaggctcctggagatcctcaagaaccccagaataatgcccacagggacaaagaag

SEQ ID NO: 24 - DNA, 30 bp, HUMAN
  exon (1): 2nd MN exon
gggatgaccagagtcattggcgctatggag

SEQ ID NO: 25 - DNA, 171 bp, HUMAN
  exon (1): 3rd MN exon
gcgacccgccctggccccgggtgtccccagcctgcgcgggccgcttccagtccccggtggatatccgcccccagctcgccgccttctgcccggccctgcgccccctggaactcctgggcttccagctcccgccgctcccagaactgcgcctgcgcaacaatggccacagtg

SEQ ID NO: 26 - DNA, 143 bp, HUMAN
  exon (1): 4th MN exon
tgcaactgaccctgcctcctgggctagagatggctctgggtcccgggcgggagtaccgggctctgcagctgcatctgcactggggggctgcaggtcgtccgggctcggagcacactgtggaaggccaccgtttccctgccgag

SEQ ID NO: 27 - DNA, 93 bp, HUMAN
  exon (1): 5th MN exon
atccacgtggttcacctcagcaccgcctttgccagagttgacgaggccttggggcgcccgggaggcctggccgtgttggccgcctttctggag

SEQ ID NO: 28 - DNA, 67 bp, HUMAN
  exon (1): 6th MN exon
gagggcccggaagaaaacagtgcctatgagcagttgctgtctcgcttggaagaaatcgctgaggaag

SEQ ID NO: 29 - DNA, 158 bp, HUMAN
  exon (1): 7th MN exon
gctcagagactcaggtcccaggactggacatatctgcactcctgccctctgacttcagccgctacttccaatatgaggggtctctgactacaccgccctgtgcccagggtgtcatctggactgtgtttaaccagacagtgatgctgagtgctaagcag

SEQ ID NO: 30 - DNA, 145 bp, HUMAN
  exon (1): 8th MN exon
ctccacaccctctctgacaccctgtggggacctggtgactctcggctacagctgaacttccgagcgacgcagcctttgaatgggcgagtgattgaggcctccttccctgctggagtggacagcagtcctcgggctgctgagccag

SEQ ID NO: 31 - DNA, 27 bp, HUMAN
  exon (1): 9th MN exon
tccagctgaattcctgcctggctgctg

SEQ ID NO: 32 - DNA, 82 bp, HUMAN
  exon (1): 10th MN exon
gtgacatcctagccctggtttttggcctcctttttgctgtcaccagcgtcgcgttccttgtgcagatgagaaggcagcacag

SEQ ID NO: 33 - DNA, 191 bp, HUMAN
  exon (1): 11th MN exon
aaggggaaccaaagggggtgtgagctaccgcccagcagaggtagccgagactggagcctagaggctggatcttggagaatgtgagaagccagccagaggcatctgagggggagccggtaactgtcctgtcctgctcattatgccacttccttttaactgccaagaaattttttaaaataaatatttataat

SEQ ID NO: 34 - DNA, 1174 bp, HUMAN
  intron (1)..(1174): 1st MN intron
gtaagtggtcatcaatctccaaatccaggttccaggaggttcatgactcccctcccataccccagcctaggctctgttcactcagggaaggaggggagactgtactccccacagaagcccttccagaggtcccataccaatatccccatccccactctcggaggtagaaagggacagatgtggagagaaaataaaaagggtgcaaaaggagagaggtgagctggatgagatgggagagaagggggaggctggagaagagaaagggatgagaactgcagatgagagaaaaaatgtgcagacagaggaaaaaaataggtggagaaggagagtcagagagtttgaggggaagagaaaaggaaagcttgggaggtgaagtgggtaccagagacaagcaagaagagctggtagaagtcatctcatcttaggctacaatgaggaattgagacctaggaagaagggacacagcaggtagagaaacgtggcttcttgactcccaagccaggaatttggggaaaggggttggagaccatacaaggcagagggatgagtggggagaagaaagaagggagaaaggaaagatggtgtactcactcatttgggactcaggactgaagtgcccactcactttttttttttttttttttgagacaaactttcacttttgttgcccaggctggagtgcaatggcgcgatctcggctcactgcaacctccacctcccgggttcaagtgattctcctgcctcagcctctagccaagtagctgcgattacaggcatgcgccaccacgcccggctaatttttgtatttttagtagagacggggtttcgccatgttggtcaggctggtctcgaactcctgatctcaggtgatccaaccaccctggcctcccaaagtgctgggattataggcgtgagccacagcgcctggcctgaagcagccactcacttttacagaccctaagacaatgattgcaagctggtaggattgctgtttggcccacccagctgcggtgttgagtttgggtgcggtctcctgtgctttgcacctggcccgcttaaggcatttgttacccgtaatgctcctgtaaggcatctgcgtttgtgacatcgttttggtcgccaggaagggattggggctctaagcttgagcggttcatccttttcatttatacag

SEQ ID NO: 35 - DNA, 193 bp, HUMAN
  intron (1)..(193): 2nd MN intron
gtgagacacccacccgctgcacagacccaatctgggaacccagctctgtggatctcccctacagccgtccctgaacactggtcccgggcgtcccacccgccgcccaccgtcccaccccctcaccttttctacccgggttccctaagttcctgacctaggcgtcagacttcctcactatactctcccaccccag

SEQ ID NO: 36 - DNA, 131 bp, HUMAN
  intron (1)..(131): 3rd MN intron
gtgagggggtctccccgccgagacttggggatggggcggggcgcagggaagggaaccgtcgcgcagtgcctgcccgggggttgggctggccctaccgggcggggccggctcacttgcctctccctacgcag

SEQ ID NO: 37 - DNA, 89 bp, HUMAN
  intron (1)..(89): 4th MN intron
gtgagcgcggactggccgagaaggggcaaaggagcggggcggacgggggccagagacgtggccctctcctaccctcgtgtccttttcag

SEQ ID NO: 38 - DNA, 1400 bp, HUMAN
  intron (1)..(1400): 5th MN intron
gtaccagatcctggacaccccctactccccgctttcccatcccatgctcctcccggactctatcgtggagccagagaccccatcccagcaagctcactcaggcccctggctgacaaactcattcacgcactgtttgttcatttaacacccactgtgaaccaggcaccagcccccaacaaggattctgaagctgtaggtccttgcctctaaggagcccacagccagtgggggaggctgacatgacagacacataggaaggacatagtaaagatggtggtcacagaggaggtgacacttaaagccttcactggtagaaaagaaaaggaggtgttcattgcagaggaaacagaatgtgcaaagactcagaatatggcctatttagggaatggctacatacaccatgattagaggaggcccagtaaagggaagggatggtgagatgcctgctaggttcactcactcacttttatttatttatttatttttttgacagtctctctgtcgcccaggctggagtgcagtggtgtgatcttgggtcactgcaacttccgcctcccgggttcaagggattctcctgcctcagcttcctgagtagctggggttacaggtgtgtgccaccatgcccagctaatttttttttgtatttttagtagacagggtttcaccatgttggtcaggctggtctcaaactcctggcctcaagtgatccgcctgactcagcctaccaaagtgctgattacaagtgtgagccaccgtgcccagccacactcactgattctttaatgccagccacacagcacaaagttcagagaaatgcctccatcatagcatgtcaatatgttcatactcttaggttcatgatgttcttaacattaggttcataagcaaaataagaaaaaagaataataaataaaagaagtggcatgtcaggacctcacctgaaaagccaaacacagaatcatgaaggtgaatgcagaggtgacaccaacacaaaggtgtatatatggtttcctgtggggagtatgtacggaggcagcagtgagtgagactgcaaacgtcagaagggcacgggtcactgagagcctagtatcctagtaaagtgggctctctccctctctctccagcttgtcattgaaaaccagtccaccaagcttgttggttcgcacagcaagagtacatagagtttgaaataatacataggattttaagagggagacactgtctctaaaaaaaaaaacaacagcaacaacaaaaagcaacaaccattacaattttatgttccctcagcattctcagagctgaggaatgggagaggactatgggaacccccttcatgttccggccttcagccatggccctggatacatgcactcatctgtcttacaatgtcattcccccag

SEQ ID NO: 39 - DNA, 1334 bp, HUMAN
  intron (1)..(1334): 6th MN intron
gtcagtttgttggtctggccactaatctctgtggcctagttcataaagaatcaccctttggagcttcaggtctgaggctggagatgggctccctccagtgcaggagggattgaagcatgagccagcgctcatcttgataataaccatgaagctgacagacacagttacccgcaaacggctgcctacagattgaaaaccaagcaaaaaccgccgggcacggtggctcacgcctgtaatcccagcactttgggaggccaaggcaggtggatcacgaggtcaagagatcaagaccatcctggccaacatggtgaaaccccatctctactaaaaatacgaaaaaatagccaggcgtggtggcgggtgcctgtaatcccagctactcgggaggctgaggcaggagaatggcatgaacccgggaggcagaagttgcagtgagccgagatcgtgccactgcactccagcctgggcaacagagcgagactcttgtctcaaaaaaaaaaaaaaaaaagaaaaccaagcaaaaaccaaaatgagacaaaaaaaacaagaccaaaaaatggtgtttggaaattgtcaaggtcaagtctggagagctaaactttttctgagaactgtttatctttaataagcatcaaatattttaactttgtaaatacttttgttggaaatcgttctcttcttagtcactcttgggtcattttaaatctcacttactctactagaccttttaggtttctgctagactaggtagaactctgcctttgcatttcttgtgtctgttttgtatagttatcaatattcatatttatttacaagttattcagatcattttttcttttcttttttttttttttttttttttttacatctttagtagagacagggtttcaccatattggccaggctgctctcaaactcctgaccttgtgatccaccagcctcggcctcccaaagtgctgggattcattttttctttttaatttgctctgggcttaaacttgtggcccagcactttatgatggtacacagagttaagagtgtagactcagacggtctttcttctttccttctcttccttcctcccttccctcccaccttcccttctctccttcctttctttcttcctctcttgcttcctcaggcctcttccagttgctccaaagccctgtacttttttttgagttaacgtcttatgggaagggcctgcacttagtgaagaagtggtctcagagttgagttaccttggcttctgggaggtgaaactgtatccctataccctgaagctttaagggggtgcaatgtagatgagaccccaacatagatcctcttcacag

SEQ ID NO: 40 - DNA, 512 bp, HUMAN
  intron (1)..(512): 7th MN intron
gtgggcctggggtgtgtgtggacacagtgggtgcgggggaaagaggatgtaagatgagatgagaaacaggagaagaaagaaatcaaggctgggctctgtggcttacgcctataatcccaccacgttgggaggctgaggtgggagaatggtttgagcccaggagttcaagacaaggcggggcaacatagtgtgaccccatctctaccaaaaaaaccccaacaaaaccaaaaatagccgggcatggtggtatgcggcctagtcccagctactcaaggaggctgaggtgggaagatcgcttgattccaggagtttgagactgcagtgagctatgatcccaccactgcctaccatctttaggatacatttatttatttataaaagaaatcaagaggctggatggggaatacaggagctggagggtggagccctgaggtgctggttgtgagctggcctgggacccttgtttcctgtcatgccatgaacccacccacactgtccactgacctccctag

SEQ ID NO: 41 - DNA, 114 bp, HUMAN
  intron (1)..(114): 8th MN intron
gtacagctttgtctggtttccccccagccagtagtcccttatcctcccatgtgtgtgccagtgtctgtcattggtggtcacagcccgcctctcacatctcctttttctctccag

SEQ ID NO: 42 - DNA, 617 bp, HUMAN
  intron (1)..(617): 9th MN intron
gtgagtctgcccctcctcttggtcctgatgccaggagactcctcagcaccattcagccccagggctgctcaggaccgcctctgctccctctccttttctgcagaacagaccccaaccccaatattagagaggcagatcatggtggggattcccccattgtccccagaggctaattgattagaatgaagcttgagaaatctcccagcatccctctcgcaaaagaatccccccccctttttttaaagatagggtctcactctgtttgccccaggctggggtgttgtggcacgatcatagctcactgcagcctcgaactcctaggctcaggcaatcctttcaccttagcttctcaaagcactgggactgtaggcatgagccactgtgcctggccccaaacggcccttttacttggcttttaggaagcaaaaacggtgcttatcttaccccttctcgtgtatccaccctcatcccttggctggcctcttctggagactgaggcactatggggctgcctgagaactcggggcaggggtggtggagtgcactgaggcaggtgttgaggaactctgcagacccctcttccttcccaaagcagccctctctgctctccatcgcag

SEQ ID NO: 43 - DNA, 130 bp, HUMAN
  intron (1)..(130): 10th MN intron
gtattacactgaccctttcttcaggcacaagcttcccccacccttgtggagtcacttcatgcaaagcgcatgcaaatgagctgctcctgggccagttttctgattagcctttcctgttgtgtacacacag

SEQ ID NO: 44 - DNA, 1401 bp, HUMAN
caaactttcacttttgttgcccaggctggagtgcaatggcgcgatctcggctcactgcaacctccacctcccgggttcaagtgattctcctgcctcagcctctagccaagtagctgcgattacaggcatgcgccaccacgcccggctaatttttgtatttttagtagagacggggtttcgccatgttggtcaggctggtctcgaactcctgatctcaggtgatccaaccaccctggcctcccaaagtgctgggattataggcgtgagccacagcgcctggcctgaagcagccactcacttttacagaccctaagacaatgattgcaagctggtaggattgctgtttggcccacccagctgcggtgttgagtttgggtgcggtctcctgtgctttgcacctggcccgcttaaggcatttgttacccgtaatgctcctgtaaggcatctgcgtttgtgacatcgttttggtcgccaggaagggattggggctctaagcttgagcggttcatccttttcatttatacaggggatgaccagagtcattggcgctatggaggtgagacacccacccgctgcacagacccaatctgggaacccagctctgtggatctcccctacagccgtccctgaacactggtcccgggcgtcccacccgccgcccaccgtcccaccccctcaccttttctacccgggttccctaagttcctgacctaggcgtcagacttcctcactatactctcccaccccaggcgacccgccctggccccgggtgtccccagcctgcgcgggccgcttccagtccccggtggatatccgcccccagctcgccgccttctgcccggccctgcgccccctggaactcctgggcttccagctcccgccgctcccagaactgcgcctgcgcaacaatggccacagtggtgagggggtctccccgccgagacttggggatggggcggggcgcagggaagggaaccgtcgcgcagtgcctgcccgggggttgggctggccctaccgggcggggccggctcacttgcctctccctacgcagtgcaactgaccctgcctcctgggctagagatggctctgggtcccgggcgggagtaccgggctctgcagctgcatctgcactggggggctgcaggtcgtccgggctcggagcacactgtggaaggccaccgtttccctgccgaggtgagcgcggactggccgagaaggggcaaaggagcggggcggacgggggccagagacgtggccctctcctaccctcgtgtccttttcagatccacgtggttcacctcagcaccgcctttgccagagttgacgaggccttggggcgcccgggaggcctggccgtgttggccgcctttctggaggtaccagatcctggacaccccctac

SEQ ID NO: 45 - protein, 59 aa, HUMAN
SSGEDDPLGEEDLPSEEDSPREEDPPGEEDLPGEEDLPGEEDLPEVKPKSEEEGSLKLE

SEQ ID NO: 46 - protein, 257 aa, HUMAN
GDDQSHWRYGGDPPWPRVSPACAGRFQSPVDIRPQLAAFCPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFP

SEQ ID NO: 47 - protein, 20 aa, HUMAN
ILALVFGLLFAVTSVAFLVQ

SEQ ID NO: 48 - protein, 25 aa, HUMAN
MRRQHRRGTKGGVSYRPAEVAETGA

SEQ ID NO: 49 - protein, 59 aa, HUMAN
SASEEPSPSEVPFPSEEPSPSEEPFPSVRPFPSVVLFPSEEPFPSKEPSPSEEPSASEE

SEQ ID NO: 50 - RNA, 470 nt, HUMAN
cauggccccgauaaccuucugccugugcacacaccugccccucacuccacccccauccuagcuuugguaugggggagagggcacagggccagacaaaccugugagacuuuggcuccaucucugcaaaagggcgcucugugagucagccugcuccccuccaggcuugcuccucccccacccagcucucguuuccaaugcacguacagcccguacacaccgugugcugggacaccccacagucagccgcauggcuccccugugccccagccccuggcucccucuguugaucccggccccugcuccaggccucacugugcaacugcugcugucacugcugcuucuggugccuguccauccccagagguugccccggaugcaggaggauucccccuugggaggaggcucuucuggggaagaugacccacugggcgaggaggaucugcccagugaagaggauucacccagagagg

SEQ ID NO: 51 - DNA, 292 bp, HUMAN
gtttttttgagacggagtcttgcatctgtcatgcccaggctggagtagcagtggtgccatctcggctcactgcaagctccacctcccgagttcacgccattttcctgcctcagcctcccgagtagctgggactacaggcgcccgccaccatgcccggctaattttttgtatttttggtagagacggggtttcaccgtgttagccagaatggtctcgatctcctgacttcgtgatccacccgcctcggcctcccaaagttctgggattacaggtgtgagccaccgcacctggc

SEQ ID NO: 52 - DNA, 262 bp, HUMAN
tttcttttttgagacagggtcttgctctgtcacccaggccagagtgcaatggtacagtctcagctcactgcagcctcaaccgcctcggctcaaaccatcatcccatttcagcctcctgagtagctgggactacaggcacatgccattacacctggctaatttttttgtatttctagtagagacagggtttggccatgttgcccgggctggtctcgaactcctggactcaagcaatccacccacctcagcctcccaaaatgag

SEQ ID NO: 53 - DNA, 2501 bp, HUMAN
  misc_feature (1)..(2501): region 5′ to transcription initiation site as determined by RNase protection assay (nucleotide 3507 of Figures 2A-2F and of SEQ ID NO:  5), corresponding to region of SEQ ID NO: 5 an region some regulatory elements are probably situated.
  unsure what base is at position 1968 (1968): unsure of base at position 1968, which is the same unknown base as that at position 1974 of SEQ ID NO. 5, i.e., the full-length MN genomic sequence, and of that unknown at position 1910.  That unknown base is in the 5′ region flanking the transcription initiation site (3507) as determined by RNase protection assay.
tgttgactcgtgaccttacccccaaccctgtgctctctgaaacatgagctgtgtccactcagggttaaatggattaagggcggtgcaagatgtgctttgttaaacagatgcttgaaggcagcatgctcgttaagagtcatcaccaatccctaatctcaagtaatcagggacacaaacactgcggaaggccgcagggtcctctgcctaggaaaaccagagacctttgttcacttgtttatctgaccttccctccactattgtccatgaccctgccaaatccccctctgtgagaaacacccaagaattatcaataaaaaaataaatttaaaaaaaaaatacaaaaaaaaaaaaaaaaaaaaaaaaagacttacgaatagttattgataaatgaatagctattggtaaagccaagtaaatgatcatattcaaaaccagacggccatcatcacagctcaagtctacctgatttgatctctttatcattgtcattctttggattcactagattagtcatcatcctcaaaattctcccccaagttctaattacgttccaaacatttaggggttacatgaagcttgaacctactaccttctttgcttttgagccatgagttgtaggaatgatgagtttacaccttacatgctggggattaatttaaactttacctctaagtcagttgggtagcctttggcttatttttgtagctaattttgtagttaatggatgcactgtgaatcttgctatgatagttttcctccacactttgccactaggggtaggtaggtactcagttttcagtaattgcttacctaagaccctaagccctatttctcttgtactggcctttatctgtaatatgggcatatttaatacaatataatttttggagtttttttgtttgtttgtttgtttgtttttttgagacggagtcttgcatctgtcatgcccaggctggagtagcagtggtgccatctcggctcactgcaagctccacctcccgagttcacgccattttcctgcctcagcctcccgagtagctgggactacaggcgcccgccaccatgcccggctaattttttgtatttttggtagagacggggtttcaccgtgttagccagaatggtctcgatctcctgacttcgtgatccacccgcctcggcctcccaaagttctgggattacaggtgtgagccaccgcacctggccaattttttgagtcttttaaagtaaaaatatgtcttgtaagctggtaactatggtacatttccttttattaatgtggtgctgacggtcatataggttcttttgagtttggcatgcatatgctactttttgcagtcctttcattacatttttctctcttcatttgaagagcatgttatatcttttagcttcacttggcttaaaaggttctctcattagcctaacacagtgtcattgttggtaccacttggatcataagtggaaaaacagtcaagaaattgcacagtaatacttgtttgtaagagggatgattcaggtgaatctgacactaagaaactcccctacctgaggtctgagattcctctgacattgctgtatataggcttttcctttgacagcctgtgactgcggactatttttcttaagcaagatatgctaaagttttgtgagcctttttccagagagaggtctcatatctgcatcaagtgagaacatataatgtctgcatgtttccatatttcaggaatgtttgcttgtgttttatgcttttatatagacagggaaacttgttcctcagtgacccaaaagaggtgggaattgttattggatatcatcattggcccacgctttctgaccttggaaacaattaagggttcataatctcaattctgtcagaattggtacaagaaatagctgctatgtttcttgacattccacttggtaggaaataagaatgtgaaactcttcagttggtgtgtgtccctngtttttttgcaatttccttcttactgtgttaaaaaaaagtatgatcttgctctgagaggtgaggcattcttaatcatgatctttaaagatcaataatataatcctttcaaggattatgtctttattataataaagataatttgtctttaacagaatcaataatataatcccttaaaggattatatctttgctgggcgcagtggctcacacctgtaatcccagcactttgggtggccaaggtggaaggatcaaatttgcctacttctatattatcttctaaagcagaattcatctctcttccctcaatatgatgatattgacagggtttgccctcactcactagattgtgagctcctgctcagggcaggtagcgttttttgtttttgtttttgtttttcttttttgagacagggtcttgctctgtcacccaggccagagtgcaatggtacagtctcagctcactgcagcctcaaccgcctcggctcaaaccatcatcccatttcagcctcctgagtagctgggactacaggcac

SEQ ID NO: 54 - DNA, 292 bp, HUMAN
  misc_feature (1)
tttttttgagacggagtcttgcatctgtcatgcccaggctggagtagcagtggtgccatctcggctcactgcaagctccacctcccgagttcacgccattttcctgcctcagcctcccgagtagctgggactacaggcgcccgccaccatgcccggctaattttttgtatttttggtagagacggggtttcaccgtgttagccagaatggtctcgatctcctgacttcgtgatccacccgcctcggcctcccaaagttctgggattacaggtgtgagccaccgcacctggcc

SEQ ID NO: 55 - DNA, 262 bp, HUMAN
ttcttttttgagacagggtcttgctctgtcacccaggccagagtgcaatggtacagtctcagctcactgcagcctcaaccgcctcggctcaaaccatcatcccatttcagcctcctgagtagctgggactacaggcacatgccattacacctggctaatttttttgtatttctagtagagacagggtttggccatgttgcccgggctggtctcgaactcctggactcaagcaatccacccacctcagcctcccaaaatgagg

SEQ ID NO: 56 - DNA, 294 bp, HUMAN
tttttttttgagacaaactttcacttttgttgcccaggctggagtgcaatggcgcgatctcggctcactgcaacctccacctcccgggttcaagtgattctcctgcctcagcctctagccaagtagctgcgattacaggcatgcgccaccacgcccggctaatttttgtatttttagtagagacggggtttcgccatgttggtcaggctggtctcgaactcctgatctcaggtgatccaaccaccctggcctcccaaagtgctgggattataggcgtgagccacagcgcctggc

SEQ ID NO: 57 - DNA, 276 bp, HUMAN
tgacagtctctctgtcgcccaggctggagtgcagtggtgtgatcttgggtcactgcaacttccgcctcccgggttcaagggattctcctgcctcagcttcctgagtagctggggttacaggtgtgtgccaccatgcccagctaatttttttttgtatttttagtagacagggtttcaccatgttggtcaggctggtctcaaactcctggcctcaagtgatccgcctgactcagcctaccaaagtgctgattacaagtgtgagccaccgtgcccagc

SEQ ID NO: 58 - DNA, 289 bp, HUMAN
cgccgggcacggtggctcacgcctgtaatcccagcactttgggaggccaaggcaggtggatcacgaggtcaagagatcaagaccatcctggccaacatggtgaaaccccatctctactaaaaatacgaaaaaatagccaggcgtggtggcgggtgcctgtaatcccagctactcgggaggctgaggcaggagaatggcatgaacccgggaggcagaagttgcagtgagccgagatcgtgccactgcactccagcctgggcaacagagcgagactcttgtctcaaaaaaa

SEQ ID NO: 59 - DNA, 298 bp, HUMAN
aggctgggctctgtggcttacgcctataatcccaccacgttgggaggctgaggtgggagaatggtttgagcccaggagttcaagacaaggcggggcaacatagtgtgaccccatctctaccaaaaaaaccccaacaaaaccaaaaatagccgggcatggtggtatgcggcctagtcccagctactcaaggaggctgaggtgggaagatcgcttgattccaggagtttgagactgcagtgagctatgatcccaccactgcctaccatctttaggatacatttatttatttataaaagaa

SEQ ID NO: 60 - DNA, 105 bp, HUMAN
ttttttacatctttagtagagacagggtttcaccatattggccaggctgctctcaaactcctgaccttgtgatccaccagcctcggcctcccaaagtgctgggat

SEQ ID NO: 61 - DNA, 83 bp, HUMAN
cctcgaactcctaggctcaggcaatcctttcaccttagcttctcaaagcactgggactgtaggcatgagccactgtgcctggc

SEQ ID NO: 62 - DNA, 11 bp, HUMAN
agaaggtaagt

SEQ ID NO: 63 - DNA, 11 bp, HUMAN
tggaggtgaga

SEQ ID NO: 64 - DNA, 11 bp, HUMAN
cagtcgtgagg

SEQ ID NO: 65 - DNA, 11 bp, HUMAN
ccgaggtgagc

SEQ ID NO: 66 - DNA, 11 bp, HUMAN
tggaggtacca

SEQ ID NO: 67 - DNA, 11 bp, HUMAN
ggaaggtcagt

SEQ ID NO: 68 - DNA, 11 bp, HUMAN
agcaggtgggc

SEQ ID NO: 69 - DNA, 11 bp, HUMAN
gccaggtacag

SEQ ID NO: 70 - DNA, 11 bp, HUMAN
tgctggtgagt

SEQ ID NO: 71 - DNA, 11 bp, HUMAN
cacaggtatta

SEQ ID NO: 72 - DNA, 11 bp, HUMAN
atacaggggat

SEQ ID NO: 73 - DNA, 11 bp, HUMAN
ccccaggcgac

SEQ ID NO: 74 - DNA, 11 bp, HUMAN
acgcagtgcaa

SEQ ID NO: 75 - DNA, 11 bp, HUMAN
tttcagatcca

SEQ ID NO: 76 - DNA, 11 bp, HUMAN
ccccaggaggg

SEQ ID NO: 77 - DNA, 11 bp, HUMAN
tcacaggctca

SEQ ID NO: 78 - DNA, 11 bp, HUMAN
ccctagctcca

SEQ ID NO: 79 - DNA, 11 bp, HUMAN
ctccagtccag

SEQ ID NO: 80 - DNA, 12 bp, HUMAN
tcgcaggtgaca

SEQ ID NO: 81 - DNA, 11 bp, HUMAN
acacagaaggg

SEQ ID NO: 82 - protein, 377 aa, HUMAN
QRLPRMQEDSPLGGGSSGEDDPLGEEDLPSEEDSPREEDPPGEEDLPGEEDLPGEEDLPEVKPKSEEEGSLKLEDLPTVEAPGDPQEPQNNAHRDKEGDDQSHWRYGGDPPWPRVSPACAGRFQSPVDIRPQLAAFCPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFPAGVDSSPRAAEPVQLNSCLAAGD

SEQ ID NO: 83 - DNA, 3532 bp, HUMAN
  misc_feature which includes the MN gene promoter (1)..(3532): region including the transcription initiation site (nucleotide 3507 of SEQ ID NO:  5 and of Figures 2A-2F) as determined by RNase protection assay, which region is inclusive of the MN of SEQ ID NO:  5 and of Figures 2A-2F.
  unsure what base is at position 1968 (1968): unsure of the base at position 1968, which is the same unknown base at position 1974 of SEQ ID NO:  5 (the full-length MN genomic sequence), position 1968 of SEQ ID NO:  58 and positit includes the transcription initiation site (nucleotide 3507 of SEQ ID NO:  5 and of Figures 2A-2F) as determined by RN ase protection assay, which region is inclusive of the MN gene promoter.
tgttgactcgtgaccttacccccaaccctgtgctctctgaaacatgagctgtgtccactcagggttaaatggattaagggcggtgcaagatgtgctttgttaaacagatgcttgaaggcagcatgctcgttaagagtcatcaccaatccctaatctcaagtaatcagggacacaaacactgcggaaggccgcagggtcctctgcctaggaaaaccagagacctttgttcacttgtttatctgaccttccctccactattgtccatgaccctgccaaatccccctctgtgagaaacacccaagaattatcaataaaaaaataaatttaaaaaaaaaatacaaaaaaaaaaaaaaaaaaaaaaaaagacttacgaatagttattgataaatgaatagctattggtaaagccaagtaaatgatcatattcaaaaccagacggccatcatcacagctcaagtctacctgatttgatctctttatcattgtcattctttggattcactagattagtcatcatcctcaaaattctcccccaagttctaattacgttccaaacatttaggggttacatgaagcttgaacctactaccttctttgcttttgagccatgagttgtaggaatgatgagtttacaccttacatgctggggattaatttaaactttacctctaagtcagttgggtagcctttggcttatttttgtagctaattttgtagttaatggatgcactgtgaatcttgctatgatagttttcctccacactttgccactaggggtaggtaggtactcagttttcagtaattgcttacctaagaccctaagccctatttctcttgtactggcctttatctgtaatatgggcatatttaatacaatataatttttggagtttttttgtttgtttgtttgtttgtttttttgagacggagtcttgcatctgtcatgcccaggctggagtagcagtggtgccatctcggctcactgcaagctccacctcccgagttcacgccattttcctgcctcagcctcccgagtagctgggactacaggcgcccgccaccatgcccggctaattttttgtatttttggtagagacggggtttcaccgtgttagccagaatggtctcgatctcctgacttcgtgatccacccgcctcggcctcccaaagttctgggattacaggtgtgagccaccgcacctggccaattttttgagtcttttaaagtaaaaatatgtcttgtaagctggtaactatggtacatttccttttattaatgtggtgctgacggtcatataggttcttttgagtttggcatgcatatgctactttttgcagtcctttcattacatttttctctcttcatttgaagagcatgttatatcttttagcttcacttggcttaaaaggttctctcattagcctaacacagtgtcattgttggtaccacttggatcataagtggaaaaacagtcaagaaattgcacagtaatacttgtttgtaagagggatgattcaggtgaatctgacactaagaaactcccctacctgaggtctgagattcctctgacattgctgtatataggcttttcctttgacagcctgtgactgcggactatttttcttaagcaagatatgctaaagttttgtgagcctttttccagagagaggtctcatatctgcatcaagtgagaacatataatgtctgcatgtttccatatttcaggaatgtttgcttgtgttttatgcttttatatagacagggaaacttgttcctcagtgacccaaaagaggtgggaattgttattggatatcatcattggcccacgctttctgaccttggaaacaattaagggttcataatctcaattctgtcagaattggtacaagaaatagctgctatgtttcttgacattccacttggtaggaaataagaatgtgaaactcttcagttggtgtgtgtccctngtttttttgcaatttccttcttactgtgttaaaaaaaagtatgatcttgctctgagaggtgaggcattcttaatcatgatctttaaagatcaataatataatcctttcaaggattatgtctttattataataaagataatttgtctttaacagaatcaataatataatcccttaaaggattatatctttgctgggcgcagtggctcacacctgtaatcccagcactttgggtggccaaggtggaaggatcaaatttgcctacttctatattatcttctaaagcagaattcatctctcttccctcaatatgatgatattgacagggtttgccctcactcactagattgtgagctcctgctcagggcaggtagcgttttttgtttttgtttttgtttttcttttttgagacagggtcttgctctgtcacccaggccagagtgcaatggtacagtctcagctcactgcagcctcaaccgcctcggctcaaaccatcatcccatttcagcctcctgagtagctgggactacaggcacatgccattacacctggctaatttttttgtatttctagtagagacagggtttggccatgttgcccgggctggtctcgaactcctggactcaagcaatccacccacctcagcctcccaaaatgagggaccgtgtcttattcatttccatgtccctagtccatagcccagtgctggacctatggtagtactaaataaatatttgttgaatgcaatagtaaatagcatttcagggagcaagaactagattaacaaaggtggtaaaaggtttggagaaaaaaataatagtttaatttggctagagtatgagggagagtagtaggagacaagatggaaaggtctcttgggcaaggttttgaaggaagttggaagtcagaagtacacaatgtgcatatcgtggcaggcagtggggagccaatgaaggcttttgagcaggagagtaatgtgttgaaaaataaatataggttaaacctatcagagcccctctgacacatacacttgcttttcattcaagctcaagtttgtctcccacatacccattacttaactcaccctcgggctcccctagcagcctgccctacctctttacctgcttcctggtggagtcagggatgtatacatgagctgctttccctctcagccagaggacatggggggccccagctcccctgcctttccccttctgtgcctggagctgggaagcaggccagggttagctgaggctggctggcaagcagctgggtggtgccagggagagcctgcatagtgccaggtggtgccttgggttccaagctagtccatggccccgataaccttctgcctgtgcacacacctgcccctcactccacccccatcctagctttggtatgggggagagggcacagggccagacaaacctgtgagactttggctccatctctgcaaaagggcgctctgtgagtcagcctgctcccctccaggcttgctcctcccccacccagctctcgtttccaatgcacgtacagcccgtacacaccgtgtgctgggacaccccac

SEQ ID NO: 84 - DNA, 204 bp, HUMAN
cctgcccctcactccacccccatcctagctttggtatgggggagagggcacagggccagacaaacctgtgagactttggctccatctctgcaaaagggcgctctgtgagtcagcctgctcccctccaggcttgctcctcccccacccagctctcgtttccaatgcacgtacagcccgtacacaccgtgtgctgggacaccccac

SEQ ID NO: 85 - DNA, 132 bp, HUMAN
ggatcctgttgactcgtgaccttacccccaaccctgtgctctctgaaacatgagctgtgtccactcagggttaaatggattaagggcggtgcaagatgtgctttgttaaacagatgcttgaaggcagcatgc

SEQ ID NO: 86 - DNA, 275 bp, HUMAN
gcatagtgccaggtggtgccttgggttccaagctagtccatggccccgataaccttctgcctgtgcacacacctgcccctcactccacccccatcctagctttggtatgggggagagggcacagggccagacaaacctgtgagactttggctccatctctgcaaaagggcgctctgtgagtcagcctgctcccctccaggcttgctcctcccccacccagctctcgtttccaatgcacgtacagcccgtacacaccgtgtgctgggacaccccac

SEQ ID NO: 87 - DNA, 89 bp, HUMAN
ctgctcccctccaggcttgctcctcccccacccagctctcgtttccaatgcacgtacagcccgtacacaccgtgtgctgggacacccca

SEQ ID NO: 88 - DNA, 61 bp, HUMAN
cacccagctctcgtttccaatgcacgtacagcccgtacacaccgtgtgctgggacacccca

SEQ ID NO: 89 - DNA, 116 bp, HUMAN
acctgcccctcactccacccccatcctagctttggtatgggggagagggcacagggccagacaaacctgtgagactttggctccatctctgcaaaagggcgctctgtgagtcagcc

SEQ ID NO: 90 - protein, 36 aa, HUMAN
GEEDLPSEEDSPREEDPPGEEDLPGEEDLPGEEDLP

SEQ ID NO: 91 - protein, 6 aa, HUMAN
GEEDLP

SEQ ID NO: 92 - protein, 4 aa, HUMAN
EEDL

SEQ ID NO: 93 - protein, 5 aa, HUMAN
EEDLP

SEQ ID NO: 94 - protein, 6 aa, HUMAN
EDLPSE

SEQ ID NO: 95 - protein, 7 aa, HUMAN
EEDLPSE

SEQ ID NO: 96 - protein, 6 aa, HUMAN
DLPGEE

SEQ ID NO: 97 - DNA, 1247 bp, HUMAN
  misc_feature (1)..(1247): region 5′ to the transcription initiation site as determined by RNase protection assay (nucleotide 3507 of SEQ ID NO:  5 and of Figures 2A-2F) in which an activating element is localiz 4 of SEQ ID NO:  5 and of Figures 2A-2F.
  unsure what base is at position 647 (647): unsure of the base at position 647, which is the same unknown base as that at position 1974 of SEQ ID NO:  5, and as that at position 1968 of SEQ ID NOS:  58 and 90.  That unknown ba localized and is 5′ to the transcription initiation site.
tatgctactttttgcagtcctttcattacatttttctctcttcatttgaagagcatgttatatcttttagcttcacttggcttaaaaggttctctcattagcctaacacagtgtcattgttggtaccacttggatcataagtggaaaaacagtcaagaaattgcacagtaatacttgtttgtaagagggatgattcaggtgaatctgacactaagaaactcccctacctgaggtctgagattcctctgacattgctgtatataggcttttcctttgacagcctgtgactgcggactatttttcttaagcaagatatgctaaagttttgtgagcctttttccagagagaggtctcatatctgcatcaagtgagaacatataatgtctgcatgtttccatatttcaggaatgtttgcttgtgttttatgcttttatatagacagggaaacttgttcctcagtgacccaaaagaggtgggaattgttattggatatcatcattggcccacgctttctgaccttggaaacaattaagggttcataatctcaattctgtcagaattggtacaagaaatagctgctatgtttcttgacattccacttggtaggaaataagaatgtgaaactcttcagttggtgtgtgtccctngtttttttgcaatttccttcttactgtgttaaaaaaaagtatgatcttgctctgagaggtgaggcattcttaatcatgatctttaaagatcaataatataatcctttcaaggattatgtctttattataataaagataatttgtctttaacagaatcaataatataatcccttaaaggattatatctttgctgggcgcagtggctcacacctgtaatcccagcactttgggtggccaaggtggaaggatcaaatttgcctacttctatattatcttctaaagcagaattcatctctcttccctcaatatgatgatattgacagggtttgccctcactcactagattgtgagctcctgctcagggcaggtagcgttttttgtttttgtttttgtttttcttttttgagacagggtcttgctctgtcacccaggccagagtgcaatggtacagtctcagctcactgcagcctcaaccgcctcggctcaaaccatcatcccatttcagcctcctgagtagctgggactacaggcacatgccattacacctggctaatttttttgtatttctagtagagacagggtttggccatgttgcccggg

SEQ ID NO: 98 - DNA, 17 bp, HUMAN
ctctgtgagtcagcctg

SEQ ID NO: 99 - DNA, 23 bp, HUMAN
aggcttgctcctcccccacccag

SEQ ID NO: 100 - DNA, 18 bp, HUMAN
agactttggctccatctc

SEQ ID NO: 101 - DNA, 20 bp, HUMAN
cactccacccccatcctagc

SEQ ID NO: 102 - DNA, 26 bp, HUMAN
gggagagggcacagggccagacaaac

SEQ ID NO: 103 - DNA, 12 bp, HUMAN
agggcacagggc

SEQ ID NO: 104 - DNA, 549 bp, HUMAN
cttgcttttcattcaagctcaagtttgtctcccacatacccattacttaactcaccctcgggctcccctagcagcctgccctacctctttacctgcttcctggtggagtcagggatgtatacatgagctgctttccctctcagccagaggacatggggggccccagctcccctgcctttccccttctgtgcctggagctgggaagcaggccagggttagctgaggctggctggcaagcagctgggtggtgccagggagagcctgcatagtgccaggtggtgccttgggttccaagctagtccatggccccgataaccttctgcctgtgcacacacctgcccctcactccacccccatcctagctttggtatgggggagagggcacagggccagacaaacctgtgagactttggctccatctctgcaaaagggcgctctgtgagtcagcctgctcccctccaggcttgctcctcccccacccagctctcgtttccaatgcacgtacagcccgtacacaccgtgtgctgggacaccccacagtcagccgca

SEQ ID NO: 105 - DNA, 8 bp, HUMAN
tacgtgca

SEQ ID NO: 106 - DNA, 50 bp, HUMAN
ctcccccacccagctctcgtttccaatgcacgtacagcccgtacacaccg

SEQ ID NO: 107 - DNA, 4 bp, HUMAN
cgtg

SEQ ID NO: 108 - DNA, 6 bp, HUMAN
acgtgc

SEQ ID NO: 109 - DNA, 10 bp, HUMAN
tacgtgcatt

SEQ ID NO: 110 - DNA, 8 bp, HUMAN
bacgtgck

SEQ ID NO: 111 - DNA, 216 bp, HUMAN
acctgcccctcactccacccccatcctagctttggtatgggggagagggcacagggccagacaaacctgtgagactttggctccatctctgcaaaagggcgctctgtgagtcagcctgctcccctccaggcttgctcctcccccacccagctctcgtttccaatgcacgtacagcccgtacacaccgtgtgctgggacaccccacagtcagccgca

SEQ ID NO: 112 - DNA, 54 bp, HUMAN
cgcgctcccccacccagctctcgtttccaatgcacgtacagcccgtacacaccg

SEQ ID NO: 113 - DNA, 54 bp, HUMAN
cgcgctcccccacccagctctcgtttccaatgcttttacagcccgtacacaccg

SEQ ID NO: 114 - DNA, 54 bp, HUMAN
cgcgctcccccacccagctctcgtttccaatgcaagtacagcccgtacacaccg

SEQ ID NO: 115 - protein, 20 aa, HUMAN
GGGGSGGGGSGGGGSGGGGS

(19) United States
(12) Patent Application Publication
(10) Pub. No.: US 2006/0084123 A1
(43) Pub. Date: Apr. 20, 2006

(54) MN and hypoxia

(76) Inventors: Adrian L. Harris, Oxford (GB); Peter J. Ratcliffe, Oxford (GB); Dirk Vordermark, Wurzburg (DE)

Correspondence Address: LEONA L. LAUDER, 235 MONTGOMERY STREET, SUITE 1026, SAN FRANCISCO, CA, 94104-0332, US

(21) Appl. No.: 11/166,997

(22) Filed: Jun. 24, 2005

Related U.S. Application Data
(63) Continuation-in-part of application No. 10/319,003, filed on Dec. 13, 2002.
(60) Provisional application No. 60/341,036, filed on Dec. 13, 2001.
(60) Provisional application No. 60/598,941, filed on Aug. 5, 2004.
(60) Provisional application No. 60/649,661, filed on Feb. 3, 2005.

(51) Int. Cl. G01N 33/574

(52) U.S. Cl. 435/7.23

(57) ABSTRACT

The MN/CA IX protein is identified herein as a hypoxia marker. The MN/CA9 gene promoter is characterized, and the location of the HIF-1 binding site within the MN/CA9 promoter is identified. Further, the hypoxia inducibility of the MN/CA9 gene and the uses of such inducibility are disclosed. In one aspect, the invention provides diagnostic/prognostic tools for determining the presence of hypoxia in a tissue in a vertebrate, preferably a human, and for measuring the relative degree of hypoxia in said vertebrate. In another aspect, the invention provides methods using tumor biopsies to predict the radioresistance of a preneoplastic/neoplastic tissue in a vertebrate subject, preferably a human patient, for diseases in which MN/CA IX levels can be used to indicate radiobiologically relevant tumor hypoxia. Such predictive methods can be used as an aid in patient therapy selection.

This application claims priority from U.S. Provisional Application Nos. 60/341,036 (filed Dec. 13, 2001), 60/598,941 (filed Aug. 5, 2004) and 60/649,661 (filed Feb. 3, 2005), and is a continuation-in-part of Zavada et al., U.S. Ser. No. 10/319,003 (filed Dec. 13, 2002).

### FIELD OF THE INVENTION

The present invention is in the general area of medical genetics and in the fields of biochemical engineering, immunochemistry and oncology. More specifically, it relates to the MN gene—a cellular gene considered to be an oncogene, known alternatively as MN/CA9, CA9, or carbonic anhydrase 9, which gene encodes the oncoprotein now known alternatively as the MN protein, the MN/CA IX isoenzyme, MN/CA IX, carbonic anhydrase IX, or the MN/G250 protein.

### BACKGROUND OF THE INVENTION

As indicated above, the MN gene and protein are known by a number of alternative names, which names are used herein interchangeably. The MN protein was found to bind zinc and have carbonic anhydrase (CA) activity and is now considered to be the ninth carbonic anhydrase isoenzyme—MN/CA IX or CA IX. [Opavsky et al., Genomics, 33: 480-487 (May 1996).] According to the carbonic anhydrase nomenclature, human CA isoenzymes are written in capital roman letters and numbers, while their genes are written in italic letters and arabic numbers. Alternatively, “MN” is used herein to refer either to carbonic anhydrase isoenzyme IX (CA IX) proteins/polypeptides, or carbonic anhydrase isoenzyme 9 (CA9) gene, nucleic acids, cDNA, mRNA etc. as indicated by the context.

The MN protein has also been identified with the G250 antigen. Uemura et al., “Expression of Tumor-Associated Antigen MN/G250 in Urologic Carcinoma: Potential Therapeutic Target,” J. Urol., 157 (4 Suppl.): 377 (Abstract 1475; 1997) states: “Sequence analysis and database searching revealed that G250 antigen is identical to MN, a human tumor-associated antigen identified in cervical carcinoma (Pastorek et al., 1994).”

Zavada et al., International Publication Number WO 93/18152 (published 16 Sep. 1993) and U.S. Pat. No. 5,387,676 (issued Feb. 7, 1995), describe the discovery and biological and molecular nature of the MN gene and protein. The MN gene was found to be present in the chromosomal DNA of all vertebrates tested, and its expression to be strongly correlated with tumorigenicity.

The MN protein was first identified in HeLa cells, derived from a human carcinoma of cervix uteri. It is found in many types of human carcinomas (notably uterine cervical, ovarian, endometrial, renal, bladder, breast, colorectal, lung, esophageal, and prostate, among others). Very few normal tissues have been found to express MN protein to any significant degree. Those MN-expressing normal tissues include the human gastric mucosa and gallbladder epithelium, and some other normal tissues of the alimentary tract. Paradoxically, MN gene expression has been found to be lost or reduced in carcinomas and other preneoplastic/neoplastic diseases in some tissues that normally express MN, e.g., gastric mucosa.

In general, oncogenesis may be signified by the abnormal expression of MN protein. For example, oncogenesis may be signified: (1) when MN protein is present in a tissue which normally does not express MN protein to any significant degree; (2) when MN protein is absent from a tissue that normally expresses it; (3) when MN gene expression is at a significantly increased level, or at a significantly reduced level from that normally expressed in a tissue; or (4) when MN protein is expressed in an abnormal location within a cell.

Zavada et al., WO 93/18152 and Zavada et al., WO 95/34650 (published 21 Dec. 1995) disclose how the discovery of the MN gene and protein and the strong association of MN gene expression and tumorigenicity led to the creation of methods that are both diagnostic/prognostic and therapeutic for cancer and precancerous conditions. Methods and compositions were provided therein for identifying the onset and presence of neoplastic disease by detecting or detecting and quantitating abnormal MN gene expression in vertebrates. Abnormal MN gene expression can be detected or detected and quantitated by a variety of conventional assays in vertebrate samples, for example, by immunoassays using MN-specific antibodies to detect or detect and quantitate MN antigen, by hybridization assays or by PCR assays, such as RT-PCR, using MN nucleic acids, such as, MN cDNA, to detect or detect and quantitate MN nucleic acids, such as, MN mRNA.

Zavada et al, WO 93/18152 and WO 95/34650 describe the production of MN-specific antibodies. A representative and preferred MN-specific antibody, the monoclonal antibody M75 (Mab M75), was deposited at the American Type Culture Collection (ATCC) in Manassus, Va. (USA) under ATCC Number HB 11128. The M75 antibody was used to discover and identify the MN protein and can be used to identify readily MN antigen in Western blots, in radioimmunoassays and immunohistochemically, for example, in tissue samples that are fresh, frozen, or formalin-, alcohol-, acetone- or otherwise fixed and/or paraffin-embedded and deparaffinized. Another representative and preferred MN-specific antibody, Mab MN12, is secreted by the hybridoma MN 12.2.2, which was deposited at the ATCC under the designation HB 11647.

Many studies have confirmed the diagnostic/prognostic utility of MN. The following articles, among others, discuss the use of the MN-specific MAb M75 in diagnosing/prognosing precancerous and cancerous cervical lesions: Leff, D. N., “Half a Century of HeLa Cells: Transatlantic Antigen Enhances Reliability of Cervical Cancer Pap Test, Clinical Trials Pending,” BioWorld® Today: The Daily Biotechnology Newspaper, 9(55) (Mar. 24, 1998); Stanbridge, E. J., “Cervical marker can help resolve ambigous Pap smears,” Diagnostics Intelligence, 10(5): 11 (1998); Liao and Stanbridge, “Expression of the MN Antigen in Cervical Papanicolaou Smears Is an Early Diagnostic Biomarker of Cervical Dysplasia,” Cancer Epidemiology, Biomarkers & Prevention, 5: 549-557 (1996); Brewer et al., “A Study of Biomarkers in Cervical Carcinoma and Clinical Correlation of the Novel Biomarker MN,” Gynecologic Oncology, 63: 337-344 (1996); and Liao et al., “Identification of the MN Antigen as a Diagnostic Biomarker of Cervical Intraepithelial Squamous and Glandular Neoplasia and Cervical Carcinomas,” American Journal of Pathology, 145(3): 598-609 (1994).

Premalignant and Malignant Colorectal Lesions. MN has been detected in normal gastric, intestinal, and biliary mucosa. [Pastorekova et al., Gastroenterology, 112: 398-408 (1997).] Immunohistochemical analysis of the normal large intestine revealed moderate staining in the proximal colon, with the reaction becoming weaker distally. The staining was confined to the basolateral surfaces of the cryptal epithelial cells, the area of greatest proliferative capacity. As MN is much more abundant in the proliferating cryptal epithelium than in the upper part of the mucosa, it may play a role in control of the proliferation and differentiation of intestinal epithelial cells. Cell proliferation increases abnormally in premalignant and malignant lesions of the colorectal epithelium, and therefore, is considered an indicator of colorectal tumor progression. [Risio, M., J. Cell Biochem, 16G: 79-87 (1992); and Moss et al., Gastroenterology, 111: 1425-1432 (1996).]

The MN protein is now considered to be the first tumor-associated carbonic anhydrase (CA) isoenzyme that has been described. Carbonic anhydrases (CAs) form a large family of genes encoding zinc metalloenzymes of great physiological importance. As catalysts of reversible hydration of carbon dioxide, these enzymes participate in a variety of biological processes, including respiration, calcification, acid-base balance, bone resorption, formation of aqueous humor, cerebrospinal fluid, saliva and gastric acid [reviewed in Dodgson et al., The Carbonic Anhydrases, Plenum Press, New York-London, pp. 398 (1991)]. CAs are widely distributed in different living organisms.

In mammals, at least seven isoenzymes (CA I-VII) and a few CA-related proteins (CARP/CA VIII, RPTP-β, RPTP-τ) had been identified [Hewett-Emmett and Tashian, Mol. Phyl. Evol., 5: 50-77 (1996)], when analysis of the MN deduced amino acid sequence revealed a striking homology between the central part of the MN protein and carbonic anhydrases, with the conserved zinc-binding site as well as the enzyme's active center. Then MN protein was found to bind zinc and to have CA activity. Based on that data, the MN protein is now considered to be the ninth carbonic anhydrase isoenzyme—MN/CA IX. [Opavsky et al., Genomics, 33: 480-487 (May 1996)]. [See also, Hewett-Emmett, supra, wherein CA IX is suggested as a nomenclatural designation.]

CAs and CA-related proteins show extensive diversity in both their tissue distribution and in their putative or established biological functions [Tashian, R. E., Adv. in Genetics, 30: 321-356 (1992)]. Some of the CAs are expressed in almost all tissues (CA II), while the expression of others appears to be more restricted (CA VI and CA VII in salivary glands). In cells, they may reside in the cytoplasm (CA I, CA II, CA III, and CA VII), in mitochondria (CA V), in secretory granules (CA VI), or they may associate with membrane (CA IV). Occasionally, nuclear localization of some isoenzymes has been noted [Parkkila et al., Gut, 35: 646-650 (1994); Parkkilla et al., Histochem. J., 27: 133-138 (1995); Mori et al., Gastroenterol., 105: 820-826 (1993)].

The CAs and CA-related proteins also differ in kinetic properties and susceptibility to inhibitors [Sly and Hu, Annu. Rev. Biochem., 64: 375-401 (1995)]. In the alimentary tract, carbonic anhydrase activity is involved in many important functions, such as saliva secretion, production of gastric acid, pancreatic juice and bile, intestinal water and ion transport, fatty acid uptake and biogenesis in the liver. At least seven CA isoenzymes have been demonstrated in different regions of the alimentary tract. However, biochemical, histochemical and immunocytochemical studies have revealed a considerable heterogeneity in their levels and distribution [Swensen, E. R., “Distribution and functions of carbonic anhydrase in the gastrointestinal tract,” In: The Carbonic Anhydrases. Cellular Physiology and Molecular Genetics, (Dodgson et al. eds.) Plenum Press, New York, pages 265-287 (1991); and Parkkila and Parkkila, Scan J. Gastroenterol., 31: 305-317 (1996)]. While CA II is found along the entire alimentary canal, CA IV is linked to the lower gastrointestinal tract, CA I, III and V are present in only a few tissues, and the expression of CA VI and VII is restricted to salivary glands [Parkkila et al., Gut, 35: 646-650 (1994); Fleming et al., J. Clin. Invest., 96: 2907-2913 (1995); Parkkila et al., Hepatology, 24: 104 (1996)].

MN/CA IX has a number of properties that distinguish it from other known CA isoenzymes and evince its relevance to oncogenesis. Those properties include its density dependent expression in cell culture (e.g., HeLa cells), its correlation with the tumorigenic phenotype of somatic cell hybrids between HeLa and normal human fibroblasts, its close association with several human carcinomas and its absence from corresponding normal tissues [e.g., Zavada et al., Int. J. Cancer, 54: 268-274 (1993); Pastorekova et al., Virology, 187: 620-626 (1992); Liao et al., Am. J. Pathol., 145: 598-609 (1994); Pastorek et al., Oncogene, 9: 2788-2888 (1994); Cote, Women's Health Weekly: News Section, p. 7 (Mar. 30, 1998); Liao et al., Cancer Res., 57: 2827 (1997); Vermylen et al., “Expression of the MN antigen as a biomarker of lung carcinoma and associated precancerous conditions,” Proceedings AACR, 39: 334 (1998); McKiernan et al., Cancer Res., 57: 2362 (1997); and Turner et al., Hum. Pathol., 28(6): 740 (1997)]. In addition, the in vitro transformation potential of MN/CA IX cDNA has been demonstrated in NIH 3T3 fibroblasts [Pastorek et al., id.].

MN and Hypoxia

MN/CA IX has been identified as a novel hypoxia regulated marker in invasive breast cancer as reported in Chia et al., “Prognostic Significance of a Novel Hypoxia Regulated Marker, Carbonic Anhydrase IX (MN/CAIX) in Invasive Breast Cancer,” Breast Cancer Research and Treatment, 64(1): 43 (November 2000). Chia et al. stated in that abstract “that MN/CA IX expression is significantly increased in hypoxic conditions across various cell lines.” MN/CA IX expression was “found to be significantly associated with a higher tumor grade (p=0.003), a negative estrogen receptor status (p<0.001) and tumor necrosis (p<0.001) . . . [and] associated with significantly worst relapse-free survival (p=0.004) and a worse overall survival (p=0.001).” [Id.]

Hypoxia is a reduction in the normal level of tissue oxygen tension. It occurs during acute and chronic vascular disease, pulmonary disease and cancer, and produces cell death if prolonged. Pathways that are regulated by hypoxia include angiogenesis, glycolysis, growth-factor signaling, immortalization, genetic instability, tissue invasion and metastasis, apoptosis and pH regulation. [Harris, A. L., Nature Reviews, 2: 38-47 (January 2002).]

Tumors become hypoxic because new blood vessels that develop in the tumors are aberrant and have poor blood flow. Although hypoxia is toxic to both tumor cells and normal cells, tumor cells undergo genetic and adaptive changes that allow them to survive and even proliferate in a hypoxic environment. These processes contribute to the malignant phenotype and to aggressive tumor behavior. Hypoxia is associated with resistance to radiation therapy and chemotherapy, but is also associated with poor outcome regardless of treatment modality, indicating that it might be an important therapeutic target. Additionally, there is a need to find an alternative to the current Eppendorf pO₂ histograph method for assessing tumor hypoxia in patients. Although the Eppendorf method provides prognostic information in a variety of tumor types, it is limited to tumors acceptable for microneedle insertion [Harris, A. L., Id.] and is an invasive technique.

The central mediator of transcriptional up-regulation of a number of genes during hypoxia is the transcription factor HIF-1. HIF-1 is a heterodimer that consists of the hypoxic response factor HIF-1α and the constitutively expressed aryl hydrocarbon receptor nuclear translocator (ARNT, also known as HIF-1β). In the absence of oxygen, HIF-1 binds to HIF-binding sites within hypoxia-response elements (HREs) of oxygen-regulated genes, thereby activating the expression of numerous hypoxia-response genes, such as erythropoietin (EPO), and the proangiogenic growth factor vascular endothelial growth factor (VEGF).

Semenza et al. PNAS (USA), 88: 5680-5684 (1991) first identified cis-activating DNA sequences that function as tissue-specific hypoxia-inducible enhancers of human erythropoietin expression. Pugh et al., PNAS (USA), 88: 10533-71 (1991) isolated such a DNA sequence 3′ to the mouse erythropoietin gene which acts as a hypoxia-inducible enhancer for a variety of heterologous promoters. Maxwell et al., PNAS (USA), 90: 2423-2427 (1993) have shown that the oxygen-sensing system which controls erythropoietin expression is widespread in mammalian cells.

McBurney et al., Nucleic Acids Res., 19: 5755-61 (1991) found that repeating the hypoxia response element (HRE) sequence, located 5′ to the hypoxia-inducible mouse phosphoglycerate kinase gene (PGK), leads to increased induction of the gene, and that using the interleukin-2 gene under tissue-specific promoters can be used for specific targeting of tumors.

Hypoxia can be used to activate therapeutic gene delivery to specific areas of tissue. Dachs et al. “Targeting gene expression to hypoxic tumor cells,” Nat. Med., 3: 515-20 (1997) has used the HRE from the mouse PGK gene promoter to drive expression of heterologous genes both in vitro and in vivo with controlled hypoxia.

For some HIF targets such as VEGF, a clear function in promoting tumor growth is established. [Kim et al., “Inhibition of vascular endothelial growth factor-induced angiogenesis suppresses tumour growth in vivo,” Nature (Lond.), 362: 841-844 (1993).] However, the full range of HIF target genes has not yet been defined, and identification of additional genes responding to this pathway is likely to provide further insights into the consequences of tumor hypoxia and HIF activation.

Indirect support for the importance of microenvironmental activation of HIF has also been provided by recent demonstrations of constitutive activation of HIF after inactivation of the VHL tumor suppressor gene. [Maxwell et al., “The tumour suppressor protein VHL targets hypoxia-inducible factors for oxygen-dependent proteolysis,” Nature (Lond.), 399: 271-275 (1999)] and amplification of the HIF response by other oncogenic mutations. [Jiang et al., “V-SRC induces expression of hypoxia-inducible factor 1 (HIF-1) and transcription of genes encoding vascular endothelial growth factor and enolase 1: involvement of HIF-1 in tumor progression,” Cancer Res., 57: 5328-5335 (1997); Blagosklonny et al., “p53 inhibits hypoxia-inducible factor-stimulated transcription,” J. Biol. Chem., 273: 11995-11998 (1998); Ravi et al., “Regulation of tumor angiogenesis by p53-induced degradation of hypoxia-inducible factor 1α,” Genes Dev., 14: 34-44 (2000); Zundel et al., “Loss of PTEN facilitates HIF-1 mediated gene expression,” Genes Dev., 14: 391-396 (2000).]

Mutations in VHL cause the familial syndrome and are also found in the majority of sporadic RCCs. [Gnarra et al., “Mutations of the VHL tumour suppressor gene in renal carcinoma,” Nat Genet., 7: 85-90 (1994).] The gene product pVHL forms part of ubiquitin-ligase complex, [Lisztwan et al., “The von Hippel-Landau tumor suppressor protein is a component of an E3 ubiquitin-protein ligase activity,” Genes Dev., 13: 1822-1833 (1999); Iwai et al., “Identification of the von Hippel-Lindau tumor-suppressor protein as part of an active E3 ubiquitin ligase complex,” Proc. Natl. Acad. Sci (USA) 96: 12436-12441 (1999)] that targets HIF-α subunits for oxygen-dependent proteolysis. [Maxwell et al., (1999) supra; Cockman et al., “Hypoxia inducible factor-α binding and ubiquitination by the von Hippel-Landau tumor suppressor protein,” J. Biol. Chem., 275: 25733-25741 (2000).]

In VHL-defective cells, HIF-α is stabilized constitutively, resulting in up-regulation of hypoxia-inducible genes such as VEGF. [Maxwell et al., (1999) supra.] Although the pVHL ubiquitin-ligase complex may have other targets [Iwai et al., supra] and other functions of pVHL have been proposed that may contribute to tumor suppressor effects [Pause et al., “The von Hippel-Lindau tumor suppressor gene is required for cell cycle exit on serum withdrawal,” Proc. Natl. Acad. Sci. (USA) 95: 993-998 (1998); Ohh et al., “The von Hippel-Landau tumor suppressor protein is required for proper assembly of an extracellular fibronectin matrix,” Mol. Cell, 1: 959-968 (1998)], these recent findings raise important questions as to the range of genes affected by constitutive HIF activation and role of such genes in oncogenesis.

In one aspect of this invention MN/CA IX is shown to be one of the most strongly hypoxia-inducible proteins. In its relationship to hypoxia, MN/CA9, considered to be an oncogene, has an interesting position as a transmembrane carbonic anhydrase (CA). CAs catalyze the reversible hydration of carbon dioxide to carbonic acid [Sly et al., Annu. Rev. Biochem., 64: 375-401 (1995)], providing a potential link between metabolism and pH regulation.

Hypoxia has been found to correlate with radioresistance of tumors, as well as tumor aggressiveness and poor prognosis [Hockel et al., Radiother. Oncol. 26: 45-50 (1993); Hockel et al., Cancer Res., 56: 4509-4515 (1996); Brizel et al., Cancer Res., 56: 941-943 (1996)]. The impact of tumor hypoxia on prognosis is most clear in head and neck tumors [Nordsmark and Overgaard, Radiother. Oncol., 57: 39-43 (2000)]. Radiobiologically relevant hypoxic cells are variously defined, but often considered to contain less than about 1% oxygen, with a half-maximal response close to 0.5% oxygen [Olive and Aquino-Parsons, Seminars in Radiation Oncology, 14(3): 241-248 (2004)]. The degree of hypoxia and the fraction of cells that are hypoxic are variable within a tumor. Hypoxic cell resistance to both chemotherapy and radiation therapy is attributed to both limited accessibility of hypoxic cells and the probability that hypoxic cells are noncycling [id.].

Evans and Koch, Cancer Letters, 195: 1-16 (2003) at page 2 graded oxygenation conditions as follows: “physiologic oxygenation as >10% oxygen, modest hypoxia as approximately 2.5% oxygen, moderate hypoxia as approximately 0.5% and severe hypoxia as approximately 0.1% oxygen. Cells that are in the moderate—modest oxygen range would be considered intermediately hypoxic.”

Hypoxia not only exists at many levels in tumors, but also may be either chronic or acute. Although it has been widely believed that acute (transient) hypoxia is more important for radiation resistance than chronic hypoxia [Denekamp and Dasu, Acta Oncol., 38: 903-918 (1999); Olive and Aquino-Parsons, (2004), supra], that hypothesis has been challenged. [See, for example, Busskink et al., Radiotherapy and Oncology 67: 3-15, 2003; Vordermark et al., Neoplasia, 3: 527-534 (2001); Vordermark et al., Radiat. Res., 159: 94-101 (2003)].

While invasive techniques (such as using Eppendorf microelectrodes) have been used to directly measure oxygen levels in tumors in order to predict radiation resistance, it would be preferable to use non-invasive techniques, such as quantitating exogenous or endogenous hypoxia markers in the initial biopsy of tumors to determine oxygen levels in tumors. Further, many tumor sites, such as intraabdominal or intracranial tumor sites, are not easily accessible by such electrodes. Evans et al. [Cancer Res., 56(2): 405-411 (1996)] studied the correlation of binding of the chemical EF5 (2-nitroimidazole) with radiation resistance of cells derived from subcutaneous rat tumors, and found a good correlation between the relative radiation resistance or hypoxic survival and EF5 binding of “moderately” hypoxic cells. A negative aspect of using exogenous markers such as EF5 or pimonidazole to predict radiation resistance of a tumor is that it requires injection of a chemical into a patient, again requiring an invasive technique beyond the initial biopsy.

Recent reviews of the search for such predictive markers of hypoxia include: Vordermark and Brown, Strahlenther. Onkol., 12: 801-811 (2003); Busskink et al. (2003), supra; Evans and Koch (2003), supra; Potter and Harris, Br. J. Cancer, 89: 2-7 (2003); Olive and Aquino-Parsons (2004), supra; and Potter and Harris, Cell Cycle, 3(2): 164-167 (2004). Most previous immunohistochemical studies correlating MN/CA IX and tissue oxygen tension [pO₂] in tumors have been retrospective patient studies. For example, in head and neck squamous cell carcinoma tissues, Beasley et al. [Cancer Res., 61: 5262-5267 (2001)] showed a median distance of 80 μm between a vessel and MN/CA IX, which corresponds to a pO₂ of about 1%. Also in 2001, Loncaster et al. [Cancer Res., 61: 6394-6399 (2001)] demonstrated Eppendorf microelectrode correlation between the hypoxic fraction of cervical carcinoma and the extent of MN/CA IX staining. Those studies did not determine whether the MN/CA IX-positive cells retained their proliferative ability, nor examined their radiation resistance.

In another retrospective study, Koukourakis et al. [Clin. Cancer Res., 7: 3399-3403 (2001)] showed a correlation between MN/CA IX expression and resistance to chemoradiotherapy in head and neck cancer patients, suggesting that MN/CA IX might be a marker of clinically important hypoxia. More recently Kaanders et al. [Cancer Res., 62: 7066-7074 (2002)] compared pimonidazole and MN/CA IX as predictors for outcome of ARCON treatment in head and neck cancer patients, and found that although the distribution of the two markers was similar, only pimonidazole correlated with patient benefit from ARCON treatment.

In 2001, Olive et al. [Cancer Res., 61: 8924-8929 (2001)] reported that single cells isolated by flow cytometry (from cervical carcinoma xenografts growing in mice) with a strong MN/CA IX signal were more radiation-resistant than cells with a weak MN/CA IX signal. However, Olive et al. [id. at page 8928] concluded that “it may not be possible to use flow cytometry to identify a ‘hypoxic’ population based on CA9 antibody binding in tumors with high hypoxic fractions . . . . CA9 expression is indicative of cells that are maximally resistant to ionizing radiation as well as those of intermediate sensitivity.” Whereas Olive et al. (Id.) measured hypoxic cells on a binary basis, Evans and Koch, Cancer Letters, 195: 1-16 (2003) at page 1 in the abstract proposed that “hypoxia should be measured as a continuum, not a binary measurement and that moderate, not severe hypoxia is of great biological consequence . . . ” (because severely hypoxic cells are destined to die).

It would be helpful to be able to identify hypoxic tumors before treatment, not only in order to identify patients with a poorer prognosis, but also to be able to predict patient response to treatments, especially radiation therapy. As there is substantial inter- and intra-tumoral heterogeneity within tumors of similar histology and site Evans and Koch [Cancer Letters, 195: 1-16 (2003)] opined that it would be important to measure hypoxia in individual patients.

The instant invention addresses those needs in the art by identifying MN as a hypoxia marker. In one aspect, MN can be used in non-invasive methods to determine the degree of hypoxia in an individual patient.

### SUMMARY OF THE INVENTION

In one aspect, the instant invention concerns the identification of MN/CA IX as one of the most strongly hypoxia-inducible proteins. Hypoxia-related MN/CA IX expression patterns indicate that it can serve as an intrinsic hypoxic marker, adding to the understanding of MN/CA IX's diagnostic and prognostic value.

Identified herein is the location of the HIF-1 consensus binding site within the MN/CA9 promoter shown in FIG. 6 (−506/+34) [SEQ ID NO: 22] and (−506/+43) [SEQ ID NO: 104] at the beginning of FIG. 1A. That HIF-1 consensus binding site within the MN/CA9 promoter is herein specified as beginning 3 bp 5′ to the transcriptional start site, oriented on the antisense strand, reading 5′-TACGTGCA-3′ [SEQ ID NO: 105] shown in FIG. 9 on the sense strand within the minimal promoter fragment (−36/+14) [SEQ ID NO: 106]. SEQ ID NO: 105 is also known as putative MN/CA9 hypoxia response element (HRE). [Wykoff et al., Cancer Res., 60: 7075-7083 (Dec. 15, 2000).]
Hypoxia-inducible factors (HIFs) locate to HIF-binding sites (HBSs) within the hypoxia-response elements (HREs) of oxygen-regulated genes . . . .
Limited O₂ supply (hypoxia) can alter the expression pattern of a specific set of genes involved in mammalian O₂ homeostasis, such as those encoding erythropoietin (EPO), transferrin or vascular endothelial growth factor (VEGF).
   [Camenisch et al., Pflügers Arch—Eur J Physiol, 443: 240-249 at 240 (2001).]

Camenisch et al. (2001) list in Table 1 at page 243 the HBSs for all genes that had been identified as direct targets of HIF-1 function including that for MN/CA IX and state at page 242: “The HIF-1 consensus DNA binding site contains CGTG [SEQ ID NO: 107] as the conserved core sequence, usually preceded by an adenosine and followed by a cytosine residue.” Such a described “usual” conserved core sequence would then read ACGTGC [SEQ ID NO: 108] which sequence is found in the HBS for MN/CA9, which is specified in Camenisch et al. to be TACGTGCATT [SEQ ID NO: 109].

Musson et al., Genetics and Epidemiology, 19(823): 62-63 (Oct. 15, 2002) point out that the transcription factor HIF-1 is known to bind to the consensus sequence 5′ (G/C/T)-ACGTGC (G/T) [SEQ ID NO: 110] within the promoter of gene which are up-regulated by HIF-1 during hypoxia. Musson et al. points out:
Hypoxia induction further requires the formation of a complex between HIF-1 and other transcription factors bound to adjacent sites . . . . In VEGF at least two sequences adjacent to the HIF-1 binding site are essential for enhanced function in hypoxia . . . . Together these sequences are known as the hypoxia response element (HRE). An AP-1 site located further downstream from the HIF-1 site has also been implicated in hypoxic VEGF regulation.

Experiments described herein delineate the nature of the MN/CA9 HRE. FIG. 6 shows some of the identified transcription factors within the MN/CA9 promoter (SEQ ID NOS: 22 and 104), and other downstream transcription sites are herein disclosed which may be significant to enhancing hypoxia induction. Ones of skill in the art will recognize transcription sites within the MN/CA9 promoter and flanking regions in view of the detailed MN/CA9 sequence information provided herein.

The MN/CA9 HRE can be considered in one sense to comprise the HIF-1 consensus binding site within the MN/C 9 promoter preferably as shown above to be SEQ ID NO: 105 and alternatively as SEQ ID NO: 109 as shown in Table 1 of Camenisch et al., supra. Variations in such HIF-1 consensus binding sites can be visualized as maintaining or promoting the hypoxia-inducible activity of the MN/CA9 promoter. For example, one of skill might visualize a nt sequence comprising the HIF-1 consensus binding site CGTG [SEQ ID NO: 107] or as ACGTGC [SEQ ID NO: 108] or as 5′(G/C/T)-ACGTGC (G/T) [SEQ ID NO: 110], among other known HIF-1 consensus binding sequences, as for example, those set forth in Table 1 of Camenisch et al., supra.

In another sense, the MN/CA9 HRE can be considered to comprise a HIF-1 consensus binding site and flanking sequences, preferably immediately adjacent [see, e.g. the MN/CA9 genomic sequence (SEQ ID NO: 5) shown in FIG. 2A-F] within which are located the binding sites of other transcription factors with which HIF-1 could form a complex thereby enhancing hypoxia induction. Preferred candidates for the location of the MN/CA9 HRE in the expanded sense of comprising additional transcription factor sites to which HIF-1 could complex include the MN/CA9 promoter [SEQ ID NOS: 22 and 104] and fragments of said promoter that comprise the HIF-1 consensus binding site, variations as described above, but preferably SEQ ID NOS: 105 and 109, more preferably SEQ ID NO: 105.

Exemplary and preferred MN/CA9 promoter fragments include the MN5 promoter fragment (−172/+31) [SEQ ID NO: 84], nearly identical to MN 5 promoter fragment (−173/+31) [SEQ ID NO: 19], closely related promoter fragment (−173/+43) [SEQ ID NO: 111], MN4 promoter fragment (−243/+31) [SEQ ID NO: 86], MN6 promoter fragment (−58/+31) [SEQ ID NO: 87], MN7 (−30/+31) [SEQ ID NO: 88], and a related minimal promoter fragment (−36/+14) [SEQ ID NO: 106]. Particularly preferred MN/CA9 promoter fragments in the HRE sense include SEQ ID NOS: 19, 84, 87, 106 and 111. The determination of the complex of HIF-1 for the MN/CA9 promoter will clarify the nature of the MN/CA9 HRE in the expanded sense.

The particularly tight regulation of MN/CA9 by hypoxia indicates that its promoter [(−506/+34) SEQ ID NO: 22 and (−506/+43) SEQ ID NO: 104] or MN promoter fragments containing a MN/CA9 HBS, wherein such MN/CA9 promoter fragments are exemplified by MN5 (−172/+31) [SEQ ID NO: 84], (−173/+31) [SEQ ID NO: 19], (−173/+43) [SEQ ID NO: 111], MN4 (−243/+31) [SEQ ID NO: 86], MN6 promoter fragment (−58/+31) [SEQ ID NO: 87], MN 7 (−30/+31) [SEQ ID NO: 88], and the related minimal promoter (−36/+14) [SEQ ID NO: 106], among many other such MN/CA9 promoter fragments, would be useful in target specific delivery systems of conditionally lethal drugs (such as enzyme converted prodrugs) in hypoxic cells. As indicated above, particularly preferred MN/CA9 promoter fragments include SEQ ID NOS: 19, 84, 87, 106 and 111, as well as related and varied promoter fragments as indicated above. The MN/CA9 promoter or MN/CA9 promoter fragments comprising the HIF-1 consensus binding sequence (varied as indicated above as long as the hypoxia inducible activity is maintained, and preferably enhanced) can be used to drive hypoxia inducibility in heterologous promoters.

Another aspect of this invention are therapeutic methods to inhibit the growth of vertebrate, preferably mammalian, more preferably human, preneoplastic or neoplastic cells in hypoxic regions of tumors, or of cells in hypoxic conditions caused other than by cancer, preferably in such cells expressing MN/CA IX at an abnormally high level. Such methods comprise transfecting such a cell with a vector comprising a nucleic acid that encodes a cytotoxic protein/polypeptide, such as HSVtk, operatively linked to the MN gene promoter or a MN gene promoter fragment that comprises the HIF-1 consensus binding site as described above. Such a MN/CA9 promoter fragment is preferably as described above and can comprise a nt sequence selected from the group consisting of, for example, SEQ ID NOS: 19, 84, 86, 87, 88, 106 and 111, and preferably the nt sequence is selected from the group consisting of SEQ ID NOS: 19, 84, 87, 106 and 111.

Such a therapeutic vector may also comprise a nucleic acid encoding a cytokine, such as, IL-2 or IFN. A variety of vectors can be visualized for therapeutic purposes including retroviral vectors among many other constructs.

A further aspect of the instant invention concerns such vectors themselves that comprise a nucleic acid that encodes a cytotoxic protein or cytotoxic polypeptide operatively linked to the MN gene promoter or a MN/CA9 promoter fragment that comprises the HIF-1 consensus binding sequence as described above, wherein said vector, when transfected into a vertebrate preneoplastic or neoplastic cell or such a cell under hypoxic conditions caused other than by cancer, preferably such a cell expressing MN/CA9 at an abnormally high level, inhibits the growth of said cell. In one preferred embodiment said cytotoxic protein is HSV thymidine kinase. Preferably, said vector further comprises a nucleic acid encoding a cytokine operatively linked to said MN gene promoter or MN/CA9 promoter fragment. In alternative and preferred embodiments, said cytokine is interferon or interleukin-2.

More specifically, one aspect of the instant invention includes: A vector comprising a nucleic acid that encodes a cytotoxic protein or cytotoxic polypeptide operatively linked to a MN/CA9 promoter or MN/CA9 promoter fragment which comprises a HIF-1 consensus binding sequence, wherein said vector, when transfected into a vertebrate cell that abnormally expresses MN/CA IX protein, such as a preneoplastic or neoplastic cell, inhibits the growth of said cell, wherein said MN/CA9 gene promoter or MN/CA9 gene promoter fragment has a nucleotide sequence selected from the group consisting of:

(a) SEQ ID NOS: 22 and 104;

(b) nucleotide sequences that are fully complementary to the nucleotide sequences of (a); and

(c) nucleotide sequences which specifically hybridize under stringent hybridization conditions of 50% formamide at 42° C. to any of the nucleotide sequences of (a) and (b). Exemplary and preferred MN/CA9 promoter fragments are set forth above.

MN/CA IX as a hypoxia marker is useful in making therapeutic decisions. For example, a cancer patient whose tumor is shown to express MN/CA IX at an abnormally high level would not be a candidate for certain kinds of chemotherapy and radiotherapy, but would be a candidate for hypoxia-selective chemotherapy.

Brown, J. M., “Exploiting the hypoxic cancer cell: mechanisms and therapeutic strategies,” Molecular Medicine Today, 6: 157-162 (April 2000) points out at page 157 that “solid tumours are considerably less well oxygenated than normal tissues. This leads to resistance to radiotherapy and anticancer chemotherapy, as well as predisposing to increased tumour metastases.” Brown explains how tumor hypoxia can be exploited in cancer treatment.

One strategy to exploit tumor hypoxia for cancer treatment proposed by Brown, id. is to use drugs that are toxic only under hypoxic conditions. Exemplary and preferred drugs that could be used under that strategy include tirapazamine and AQ4N, a di-N-oxide analogue of mitozantrome.

A second mode of exploiting hypoxia proposed by Brown, id. is by gene therapy strategies developed to take advantage of the selective induction of HIF-1. Brown notes that a tumor-specific delivery system can be developed wherein a promoter that is highly responsive to HIF-1 would drive the expression of a conditionally lethal gene under hypoxic but not normoxic conditions. “Expression of an enzyme not normally found in the human body could, under the control of a hypoxia-responsive promoter, convert a nontoxic pro-drug into a toxic drug in the tumour.” [Brown, id., page 160.] Exemplary is the use of the bacterial cytosine deaminase, which converts the nontoxic 5-fluorocytosine to the anticancer drug 5-fluorouracil (5FU) cited by Brown to Trinh et al., Cancer Res., 55: 4808-4812 (1995).

Ratcliffe et al., U.S. Pat. Nos. 5,942,434 and 6,265,390 explain how anti-cancer drugs become activated under hypoxia [Workman and Stafford, Cancer and Metastasis Reviews, 12: 73-82 (1993)], but that the use of a drug activation system, wherein the enzyme that activates the drug is significantly increased under hypoxia, results in much enhanced therapeutic effect. Ratcliffe et al., supra in the last five paragraphs in the Summary of the Invention states:
The invention provides a nucleic acid construct comprising at least one gene encoding a species having activity against disease, operatively linked to a hypoxically inducible expression control sequence. When the construct is present in a suitable host cell, expression of the gene will thus be regulated according to the level of oxygenation. Preferably the expression control sequence is a promoter or enhancer. In a host cell under hypoxic conditions, expression of the gene will be initiated or upregulated, while under conditions of normoxia (normal oxygen level) the gene will be expressed at a lower level or not expressed at all. The expression level may vary according to the degree of hypoxia. Thus, a gene product which has therapeutic activity can be targeted to cells affected by disease, eg. tumour cells. The species encoded by the gene in the construct according to the invention may be for example a cytokine, such as interleukin-2 (IL-2) which is known to be active in the immune response against tumours. Genes encoding other molecules which have an anti-tumour effect may also be used. In a preferred embodiment of the construct according to the invention, the species encoded by the gene is a pro-drug activation system, for example the thymidine phosphorylase enzyme, which converts a relatively inactive drug into a much more potent one. Transfection of the thymidine phosphorylase gene into human breast cancer cells has been shown to greatly increase the sensitivity of the cancer cells to 5-deoxy-5FU . . . . The thymidine phosphorylase gene has not previously been reported as an agent for gene therapy. Another pro-drug activation system which can be used is cytosine deaminase, which activates the pro-drug 5-fluorocytosine (5-FC) to form the antitumour agent 5-fluorouracil (5-FU). A further example of a pro-drug activation system for use in the invention is cytochrome p450 to activate the drug SR4233 (Walton et al, [Biochem. Pharmacol, 44: 251-259] 1992).
The construct according to the invention may contain more than one gene and more than one type of gene. Additional genes may encode further species having activity against disease, or they may have gene products with other activities.

In one aspect, the present invention provides diagnostic/prognostic tools for determining the presence of hypoxia in a tissue in an animal, preferably a vertebrate, more preferably a mammal, still more preferably a human, and for measuring the relative degree of hypoxia in said animal.

In still another aspect, the present invention provides for immunoassays to determine the degree of hypoxia in, and/or predict the radioresistance of, a preneoplastic/neoplastic tissue in a vertebrate subject. Related methods to detect cells that are both hypoxic and metabolically active in such preneoplastic/neoplastic tissue are also provided.

In another aspect, the present invention provides tools for gene therapy designed to exploit hypoxic conditions therapeutically.

In still another aspect, the present invention provides prognostic tools for patients with diseases associated with hypoxic conditions.

In one embodiment, the present invention provides for an expression vector to determine the presence of hypoxia in a tissue in an animal. In another embodiment, the present invention provides for an expression vector to determine the relative degree of hypoxia in a tissue of an animal.

In one aspect, the invention is directed to the MN/CA9 hypoxia-response element (HRE) and MN/CA9 promoter fragments comprising said HRE including the MN/CA9 HIF-1 consensus binding sequence or a variation thereof, preferably also comprising elements to enhance hypoxia inducibility. The MN/CA9 HRE has several utilities. For example, the MN/CA9 HRE or MN/CA9 promoter fragments comprising said MN/CA9 HRE or a fragment of said MN/CA9 HRE, for example, at least the MN/CA9 HIF-1 consensus binding sequence (HBS), can be inserted into a suitable expression vector, in combination with, preferably within, a promoter or promoter fragment operatively linked to a gene, preferably a gene's coding region. Cells can be transformed with such an expression vector, and the protein expressed therein will be regulated according to the degree of oxygenation. Under hypoxia, gene expression will be initiated or increased; under conditions of normoxia, gene expression will be reduced or eliminated.

This invention also concerns recombinant nucleic acid molecules that comprise a MN/CA9 HRE or a MN/CA9 promoter fragment comprising said MN/CA9 HRE or a MN/CA9 HIF-1 consensus binding sequence. Said recombinant nucleic acid molecules may also comprise a nucleic acid sequence that encodes a non-MN/CA IX protein or polypeptide, and/or a non-MN/CA9 HRE, a non-MN/CA9 HBS, a non-MN/CA9 promoter or promoter fragment, and one or more enhancer elements (that enhance hypoxia inducibility). Examples of a coding sequence for a non-MN/CA9 protein/polypeptide include the DNA sequence coding for the luciferase gene, the alpha-peptide coding region of beta-galactosidase, and a sequence coding for glutathione S-transferase. Further, claimed herein are such recombinant fusion proteins/polypeptides which are substantially pure and non-naturally occurring.

According to one aspect of the invention, a gene regulated by the MN/CA9 HRE, or by a MN/CA9 promoter fragment containing a MN/CA9 HRE or HBS, in the vector may encode for a cytokine, such as interleukin-2, or other molecules with known anti-tumor effects.

In a preferred embodiment, the gene regulated by the MN/CA9 HRE or by MN/CA9 promoter fragment comprising a MN/CA9 HRE or HBS encodes for a pro-drug activation system, such as the thymidine phosphorylase enzyme, which converts an inactive drug into an active one. Other pro-drug activation systems according to the invention are cytosine deaminase, which activates the pro-drug 5-flyorocytosine (5-FC) to form the antitumor drug 5-fluorouracil (5-FU), and cytochrome p450 to activate the drug SR4233.

Host cells transformed with the constructs of this invention are also encompassed within the scope of the invention.

Also disclosed herein are methods to use the MN/CA9 gene and nucleic acid fragments thereof, including the herein described MN/CA9 promoter and promoter fragments, particularly those comprising the MN/CA9 HRE (preferably enhanced) and/or HIF-1 consensus binding sequence, MN/CA IX proteins/polypeptides, MN/CA IX-specific antibodies, whether monoclonal, polyclonal and/or antibody fragments, to identify hypoxic conditions, whether chronic or acute, particularly chronic, and/or to target therapeutic drugs, including for example, enzyme activated pro-drugs, cytotoxic proteins/polypeptides, lethal drugs (preferably conditionally lethal that is, for example, lethal under hypoxic conditions, or only expressed under hypoxic conditions) to hypoxic tissues or cells.

Further provided are other therapeutic methods wherein the growth of a vertebrate, preferably mammalian, more preferably human, preneoplastic or neoplastic cell that abnormally expresses MN protein is inhibited. Said methods comprise transfecting said cell with a vector comprising an expression control sequence operatively linked to a nucleic acid encoding the variable domains of an MN-specific antibody, wherein said domains are separated by a flexible linker peptide, preferably SEQ ID NO: 115. Preferably said expression control sequence comprises the MN gene promoter or a MN/CA9 promoter fragment comprising a HIF-1 consensus sequence as described above.

Still another aspect of the instant invention is a vector comprising an expression control sequence operatively linked to a nucleic acid encoding the variable domains of a MN-specific antibody, wherein said domains are separated by a flexible linker polypeptide, and wherein said vector, when transfected into a vertebrate preneoplastic or neoplastic cell that abnormally expresses MN protein, inhibits the growth of said cell. Preferably said expression control sequence comprises the MN gene promoter or a MN/CA9 promoter fragment, preferably comprising the HIF-1 consensus binding sequence as described above, operatively linked to said nucleic acid. Further preferably, said flexible linker polypeptide has the amino acid sequence of SEQ ID NO: 115, and even further preferably, said MN gene promoter has the nucleotide sequence of SEQ ID NO: 22.

A further aspect of the instant invention is a method to determine the degree of hypoxia in a tissue, preferably a preneoplastic/neoplastic tissue, in a subject vertebrate, preferably mammalian, still more preferably human. Said method comprises isolating a sample from said tissue from said vertebrate, and immunologically detecting and quantifying the level of MN/CA IX protein/polypeptide in said tissue, wherein the level of said MN/CA IX protein/polypeptide found in said tissue, relative to the level of MN/CA IX protein/polypeptide found at 0.1% O₂ in comparable cells isolated from an organism of the same taxonomic classification as the subject vertebrate, indicates the degree of hypoxia in said tissue.

A related aspect of the invention is a method to detect cells that are both hypoxic and metabolically active, comprising immunologically detecting and quantifying the level of MN/CA IX protein/polypeptide in a preneoplastic/neoplastic tissue derived from a subject vertebrate, wherein if detectable MN/CA IX protein/polypeptide is found, concluding that the cells in the vertebrate sample are both hypoxic and metabolically active; or if no detectable MN/CA IX protein/polypeptide is found, concluding that the cells in the vertebrate sample are not hypoxic and/or not metabolically active. If a second hypoxic marker indicates that the cells in the vertebrate sample are hypoxic, the conclusion can be made that said cells are hypoxic but not metabolically active.

Further provided are methods to predict the radioresistance of an affected tissue in a subject vertebrate, preferably mammalian, still more preferably human, with a preneoplastic/neoplastic disease. Such methods comprise two assays: 1) a preliminary in vitro screening assay which correlates MN/CA IX levels with levels of hypoxia and degrees of radioresistance found in preneoplastic/neoplastic cells comparable to the patient tissue; and 2) a subsequent quantitative immunoassay of MN/CA IX levels in a patient tissue sample which is used to extrapolate a predicted radioresistance from the correlation found in the preliminary assay.

One method of predicting the degree of radioresistance of an affected tissue in a subject vertebrate with a preneoplastic/neoplastic disease, wherein MN/CA IX protein/polypeptide levels in said preneoplastic/neoplastic tissue can be used to indicate radiobiologically relevant tumor hypoxia in said tissue, comprises the steps of:

(a) performing an in vitro test of comparable preneoplastic/neoplastic cells, correlating the MN/CA IX protein/polypeptide levels in said cells with degrees of cellular radioresistance, wherein said cells are isolated from an organism of the same taxonomic classification as the subject vertebrate;

(b) isolating a sample from the affected tissue in said subject vertebrate;

(c) immunologically detecting and quantitating the MN/CA IX protein/polypeptide level in said vertebrate sample; and

(d) predicting the degree of radioresistance of the subject vertebrate tissue by comparing the MN/CA IX protein/polypeptide level found in step (c) with the MN/CA IX protein/polypeptide levels of step (a), and extrapolating therefrom a predicted degree of radioresistance of the subject vertebrate tissue. Said correlating step (a) comprises correlating MN/CA IX protein/polypeptide levels with hypoxic radiation resistance in said comparable preneoplastic/neoplastic cells, preferably by determining the oxygen enhancement ratio (OER), still more preferably, by determining the modified oxygen enhancement ratio (OER′) in said comparable cells.

The prenoplastic/neoplastic cells comparable to the patient tissue used in the correlating step (a) can be cells isolated from an organism of the same taxonomic classification as the subject vertebrate, or they can be cells isolated from the subject vertebrate. For example, the preneoplastic/neoplastic disease can be head and neck cancer, and said comparable cells can be FaDu human pharyngeal carcinoma cells, or they can be from a biopsy from the patient tumor.

Further, the patient tissue sample used in the detecting and quantitating step (c) can be a biopsy from the patient, preferably a formalin-fixed, paraffin-embedded tissue sample. Said patient tissue sample or biopsy can be taken a patient tumor and/or from a metastatic lesion derived from said tumor.

Said methods can be used as an aid in the selection of treatment for said preneoplastic/neoplastic disease in the subject vertebrate. For example, if the predicted degree of radioresistance of the affected vertebrate tissue is high, the decision can be made not to use radiation therapy; or if the predicted degree of radioresistance of the affected vertebrate tissue is low, the decision can be made to use radiation therapy.

Said preneoplastic/neoplastic disease afflicting said subject vertebrate can be, among other preneoplastic/neoplastic diseases, preneoplastic/neoplastic diseases of head and neck, mammary, urinary tract, kidney, bladder, ovarian, uterine, cervical, endometrial, vaginal, vulvar, prostate, liver, lung, skin, thyroid, pancreatic, testicular, brain, gastrointestinal, colon, colorectal and mesodermal tissues; preferably, said preneoplastic/neoplastic disease is head and neck cancer or precancer.

Methods to predict the radioresistance of an affected tissue in a subject vertebrate also comprise the use of immunoassays to quantitative MN/CA IX levels, both in the preliminary in vitro screening assay of prenoplastic/neoplastic cells comparable to the patient tissue, and the subsequent quantitative immunoassay of MN/CA IX levels in the patient tissue. Such immunoassays can be, among other immunoassay formats, Western blots, enzyme-inked immunosorbent assays, radioimmunoassays, competition immunoassays, dual antibody sandwich assays, immunohistochemical staining assays, agglutination assays, and fluorescent immunoassays. Preferably, said immunoassays comprise the use of the M75 monoclonal antibody secreted by the hybridoma VU-M75 which has Accession No. ATCC HB 11128. Still more preferably, said correlating step comprises the use of a Western blot, and said detecting and quantitating step comprises the use of an immunohistochemical staining assay. More preferably, said detecting and quantitating step further comprises determining the percentage of MN/CA IX immunoreactive cells and/or the intensity of immunostaining of immunoreactive cells.

### Abbreviations

The following abbreviations are used herein:
5-FC—5-flyorocytosine
5-FU—5-fluorouracil
aa—amino acid
ARCON—accelerated radiotherapy with carbogen and nicotinamide
ATCC—American Type Culture Collection
bp—base pairs
BLV—bovine leukemia virus
BSA—bovine serum albumin
BRL—Bethesda Research Laboratories
CA—carbonic anhydrase
CAM—cell adhesion molecule
CARP—carbonic anhydrase related protein
CAT—chloramphenicol acetyltransferase
Ci—curie
cm—centimeter
CMV—cytomegalovirus
cpm—counts per minute
C-terminus—carboxyl-terminus
CTL—cytotoxic T lymphocytes
° C.—degrees centigrade
DEAE—diethylaminoethyl
DFO—desferrioxamine
DMEM—Dulbecco modified Eagle medium
ds—double-stranded
EDTA—ethylenediaminetetraacetate
EGF—epidermal growth factor
EIA—enzyme immunoassay
ELISA—enzyme-linked immunosorbent assay
EMSA—electrophoretic mobility shift assay
EPO—erythropoietin
F—fibroblasts
FACS—fluorescence-activated cell sorting
FCS—fetal calf serum
FITC—fluorescein isothiocyanate
FTP—DNase 1 footprinting analysis
GFP—green fluorescent protein
GST-MN—fusion protein MN glutathione S-transferase
GVC—ganciclovir
h—hour
H—HeLa cells
HBS—HIF-binding site
H-E—haematoxylin-eosin
HEF—human embryo fibroblasts
HeLa K—standard type of HeLa cells
HeLa S—Stanbridge's mutant HeLa D98/AH.2
H/F-T—hybrid HeLa fibroblast cells that are tumorigenic; derived from HeLa D98/AH.2
H/F-N—hybrid HeLa fibroblast cells that are nontumorigenic; derived from HeLa D98/AH.2
HIF—hypoxia-inducible factor
HPV—Human papilloma virus
HRE—hypoxia response element
HRP—horseradish peroxidase
HSV—Herpes simplex virus
IC—intracellular
IFN—interferon
IL-2—interleukin-2
Inr—initiator
IPTG—isopropyl-beta-D-thiogalacto-pyranoside
kb—kilobase
kbp—kilobase pairs
kd or kDa—kilodaltons
KS—keratan sulphate
LCMV—lymphocytic choriomeningitis virus
LTR—long terminal repeat
M—molar
mA—milliampere
MAb—monoclonal antibody
MCSF—macrophage colony stimulating factor
ME—mercaptoethanol
MEM—minimal essential medium
min.—minute(s)
mg—milligram
ml—milliliter
mM—millimolar
MMC—mitomycin C
mmol—millimole
MLV—murine leukemia virus
N—normal concentration
NEG—negative
ng—nanogram
nm—nanometer
nt—nucleotide
N-terminus—amino-terminus
ODN—oligodeoxynucleotide
OER—oxygen enhancement ratio
OER′—modified oxygen enhancement ratio
ORF—open reading frame
PA—Protein A
PBS—phosphate buffered saline
PCR—polymerase chain reaction
PEST—combination of one-letter abbreviations for proline, glutamic acid, serine, threonine
PG—proteoglycan
PGK—phosphoglycerate kinase
pI—isoelectric point
PMA—phorbol 12-myristate 13-acetate
POS—positive
Py—pyrimidine
RACE—rapid amplification of cDNA ends
RCC—renal cell carcinoma
RIA—radioimmunoassay
RIP—radioimmunoprecipitation
RIPA—radioimmunoprecipitation assay
RNP—RNase protection assay
RT-PCR—reverse transcription polymerase chain reaction
SAC—Staphylococcus aureus cells
S. aureus—Staphylococcus aureus
sc—subcutaneous
SDRE—serum dose response element
SDS—sodium dodecyl sulfate
SDS-PAGE—sodium dodecyl sulfate-polyacrylamide gel electrophoresis
SINE—short interspersed repeated sequence
SP—signal peptide
SP-RIA—solid-phase radioimmunoassay
SSDS—synthetic splice donor site
SSH—subtractive suppressive PCR
SSPE—NaCl (0.18 M), sodium phosphate (0.01 M), EDTA (0.001 M)
SV40—simian virus 40
TBE—Tris-borate/EDTA electrophoresis buffer
TC—tissue culture
TCA—trichloroacetic acid
TC media—tissue culture media
TC—tissue culture
tk—thymidine kinase
TM—transmembrane
TMB—tetramethylbenzidine
Tris—tris (hydroxymethyl) aminomethane
μCi—microcurie
μg—microgram
μl—microliter
μM—micromolar
VEGF—vascular endothelial growth factor
VSV—vesicular stomatitis virus
VV—vaccinia virus
X-MLV—xenotropic murine leukemia virus

### Cell Lines

AGS—cell line derived from a primary adenogastric carcinoma [Barranco and Townsend, Cancer Res., 43: 1703 (1983) and Invest. New Drugs, 1: 117 (1983)]; available from the ATCC under CRL-1739;
BL-3—bovine B lymphocytes [ATCC CRL-8037; leukemia cell suspension; J. Natl. Cancer Inst. (Bethesda) 40: 737 (1968)];
C33—a cell line derived from a human cervical carcinoma biopsy [Auersperg, N., J. Nat'l. Cancer Inst. (Bethesda), 32: 135-148 (1964)]; available from the ATCC under HTB-31;
C33A—human cervical carcinoma cells [ATCC HTB-31; J. Natl. Cancer Inst. (Bethesda) 32: 135 (1964)];
C4.5—CHO wild-type, parental to Ka13, the same cell line as that described in Wood et al., J. Biol. Chem., 273: 8360-8368 (1998);
COS—simian cell line [Gluzman, Y., Cell, 23: 175 (1981)];
HeLa—from American Type Culture Collection (ATCC)
HeLa K—standard type of HeLa cells; aneuploid, epithelial-like cell line isolated from a human cervical adenocarcinoma [Gey et al., Cancer Res., 12: 264 (1952); Jones et al., Obstet. Gynecol., 38: 945-949 (1971)] obtained from Professor B. Korych, [Institute of Medical Microbiology and Immunology, Charles University; Prague, Czech Republic];
HeLa D98/AH.2 (also HeLa s)—Mutant HeLa clone that is hypoxanthine guanine phosphoribosyl transferase-deficient (HGPRT) kindly provided by Eric J. Stanbridge [Department of Microbiology, College of Medicine, University of California, Irvine, Calif. (USA)] and reported in Stanbridge et al., Science, 215: 252-259 (15 Jan. 1982); parent of hybrid cells H/F-N and H/F-T, also obtained from E. J. Stanbridge;
Ka13—CHO mutant cell functionally defective for the HIF-1α subunit, the same cell line as that described in Wood et al. (1998), supra;
KATO III—cell line prepared from a metastatic form of a gastric carcinoma [Sekiguichi et al., Japan J. Exp. Med., 48: 61 (1978)]; available from the ATCC under HTB-103;
NIH-3T3—murine fibroblast cell line reported in Aaronson, Science, 237: 178 (1987);
QT35—quail fibrosarcoma cells [ECACC: 93120832; Cell, 11: 95 (1977)];
Raj—human Burkitt's lymphoma cell line [ATCC CCL-86; Lancet, 1: 238 (1964)];
Rat2TK⁻—cell line (rat embryo, thymidine kinase mutant) was derived from a subclone of a 5′-bromo-deoxyuridine resistant strain of the Fischer rat fibroblast 3T3-like cell line Rat1; the cells lack appreciable levels of nuclear thymidine kinase [Ahrens, B., Virology, 113: 408 (1981)];
SiHa—human cervical squamous carcinoma cell line [ATCC HTB-35; Friedl et al., Proc. Soc. Exp. Biol. Med. 135: 543 (1990)];
XC—cells derived from a rat rhabdomyosarcoma induced with Rous sarcoma virus-induced rat sarcoma [Svoboda, J., Natl. Cancer Center Institute Monograph No. 17, IN: “International Conference on Avian Tumor Viruses” (J. W. Beard ed.), pp. 277-298 (1964)], kindly provided by Jan Svoboda [Institute of Molecular Genetics, Czechoslovak Academy of Sciences; Prague, Czech Republic]; and
CGL1—H/F-N hybrid cells (HeLa D98/AH.2 derivative);
CGL2—H/F-N hybrid cells (HeLa D98/AH.2 derivative);
CGL3—H/F-T hybrid cells (HeLa D98/AH.2 derivative);
CGL4—H/F-T hybrid cells (HeLa D98/Ah.2 derivative).

### Nucleotide and Amino Acid Sequence Symbols

The following symbols are used to represent nucleotides herein:

Base

| Symbol | Meaning |
|---|---|
| A | adenine |
| C | cytosine |
| G | guanine |
| T | thymine |
| U | uracil |
| I | inosine |
| M | A or C |
| R | A or G |
| W | A or T/U |
| S | C or G |
| Y | C or T/U |
| K | G or T/U |
| V | A or C or G |
| H | A or C or T/U |
| D | A or G or T/U |
| B | C or G or T/U |
| N/X | A or C or G or T/U |

There are twenty main amino acids, each of which is specified by a different arrangement of three adjacent nucleotides (triplet code or codon), and which are linked together in a specific order to form a characteristic protein. A three-letter or one-letter convention is used herein to identify said amino acids, as, for example, in FIG. 1 as follows:

| | 3 Ltr. | 1 Ltr. |
|---|---|---|
| Amino acid name | Abbrev. | Abbrev. |
| Alanine | Ala | A |
| Arginine | Arg | R |
| Asparagine | Asn | N |
| Aspartic Acid | Asp | D |
| Cysteine | Cys | C |
| Glutamic Acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Unknown or other | | X |

### BRIEF DESCRIPTION OF THE FIGURES

FIG. 1A-C provides the nucleotide sequence for a MN cDNA [SEQ ID NO: 1] clone isolated as described herein. FIG. 1 A-C also sets forth the predicted amino acid sequence [SEQ ID NO: 2] encoded by the cDNA.

FIG. 2A-F provides a 10,898 bp complete genomic sequence of MN [SEQ ID NO: 5]. The base count is as follows: 2654 A; 2739 C; 2645 G; and 2859 T. The 11 exons are in general shown in capital letters, but exon 1 is considered to begin at position 3507 as determined by RNase protection assay.

FIG. 3 is a restriction map of the full-length MN cDNA. The open reading frame is shown as an open box. The thick lines below the restriction map illustrate the sizes and positions of two overlapping cDNA clones. The horizontal arrows indicate the positions of primers R1 [SEQ ID NO: 7] and R2 [SEQ ID NO: 8] used for the 5′ end RACE. Relevant restriction sites are BamHI (B), EcoRV (V), EcoRI (E), PstI (Ps), PvuII (Pv).

FIG. 4 schematically represents the 5′ MN genomic region of a MN genomic clone wherein the numbering corresponds to transcription initiation sites estimated by RACE.

FIG. 5 provides an exon-intron map of the human MN/CA IX gene. The positions and sizes of the exons (numbered, cross-hatched boxes), Alu repeat elements (open boxes) and an LTR-related sequence (first unnumbered stippled box) are adjusted to the indicated scale. The exons corresponding to individual MN/CA IX protein domains are separated by dashed lines designated PG (proteoglycan-like domain), CA (carbonic anhydrase domain), TM (transmembrane anchor) and IC (intracytoplasmic tail). Below the map, the alignment of amino acid sequences illustrates the extent of homology between the MN/CA IX protein PG region (aa 53-111) [SEQ ID NO: 45] and the human aggrecan (aa 781-839) [SEQ ID NO: 49].

FIG. 6 is a nucleotide sequence for the proposed promoter of the human MN gene [SEQ ID NO: 22]. The nucleotides are numbered from the transcription initiation site according to RNase protection assay. Potential regulatory elements are overlined. Transcription start sites are indicated by asterisks (RNase protection) and dots (RACE) above the corresponding nucleotides. The sequence of the 1st exon begins under the asterisks. FTP analysis of the MN4 promoter fragment revealed 5 regions (I-V) protected at both the coding and noncoding strands, and two regions (VI and VII) protected at the coding strand but not at the noncoding strand.

FIG. 7 provides a schematic of the alignment of MN genomic clones according to their position related to the transcription initiation site. All the genomic fragments except Bd3 were isolated from a lambda FIX II genomic library derived from HeLa cells. Clone Bd3 was derived from a human fetal brain library.

FIG. 8 schematically represents the MN protein structure. The abbreviations are the same as used in FIG. 5. The scale indicates the number of amino acids.

FIG. 9 describes the functional analysis of human MN/CA9 5′-flanking sequences in transient expression assays. Left panel, schematic diagram of reporter genes; the indicated MN/CA9 wild-type and mutant sequences were inserted 5′ to a promoterless luciferase reporter gene. Arrow, 5′ transcriptional initiation site. Underlined sequence, the MN/CA9 HIF-1 consensus binding sequence [SEQ ID NO: 105] within the MN/CA9 HRE or considered to be the putative MN/CA9 HRE [Wykoff et al., Cancer Res., 60: 7075-7083 (Dec. 15, 2000)], whereas MN/CA9 promoter fragments may comprise enhancer elements with which the HIF-1 transcription factor can complex. Right panels, reporter gene activities in transiently transfected cells. The MN/CA9 promoter sequences are indicated to the left of each column. SV-40, control minimal SV-40 promoter. A, activities in normoxic and hypoxic HeLa cells. B and C, activities in wild-type CHO(C4.5) cells (columns 1) and HIF-1 α-deficient CHO (Ka13) cells (columns 2). A, hypoxia-inducible activity of the MN/CA9 promoter. B, hypoxia-inducible activity of the MN/CA9 promoter is ablated in Ka13 cells. Cotransfection of HIF-1 α restores induction by hypoxia in Ka13 cells and augments MN/CA9 promoter activity in both wild-type and Ka13 cells. In comparison, minimal effects are seen on the SV40 promoter. C, a minimal MN/CA9 promoter [SEQ ID NO: 106] retains HIF-1α-dependent, hypoxia inducible activity. Two mutations within the putative MN/CA9 HRE or MN/CA9 HIF-1 consensus binding sequence, MUT1 and MUT2, completely ablate hypoxia-inducible activity, whereas basal transcription is preserved. Columns, mean luciferase activities corrected for transfection efficiency from a typical experiment performed in duplicate. Each duplicate experiment was repeated two to six times. Numbers to the right are the ratios of hypoxic to normoxic expression of the indicated reporter construct. Transfected cells were incubated at 20% O₂ for 8 h and then incubated at 20% O₂ (normoxia) or 0.1% O₂ (hypoxia) for 16 h. [FIG. 2 of Wykoff et al., Cancer Res., 60: 7075-7083 (Dec. 15, 2000).]

FIG. 10A-B describes the time course of carbonic anhydrase IX (MN/CA IX) protein accumulation under hypoxic conditions in vitro. Human HT 1080 fibrosarcoma and FaDu pharyngeal carcinoma cells were subjected to 5%, 1% or 0.1% O₂ for various durations, and MN/CA IX protein was determined in whole-cell lysates using Western blot analysis (β-actin served as loading control for quantification of MN/CA IX). Treatment with 100 μM desferrioxamine (DFO) served as positive control. Following 24 h of hypoxia, stability upon reoxygenation was also examined. Comparison of different O₂ concentrations indicates maximal MN/CA IX protein already at 5% O₂ in HT 1080 and half-maximal MN/CA IX at this concentration in FaDu (n=3, means±SEM).

FIG. 11A-B depicts the effect of long reoxygenation times following 24 h of hypoxia at 0.1% O₂ in HT 1080 human fibrosarcoma and FaDu human pharyngeal carcinoma cells. MN/CA IX protein was determined in whole-cell lysates, using Western blot analysis with β-actin as loading control. Control cells were analyzed at the respective time points following 24 h in normoxia. Quantitative analysis indicates stability of MN/CA IX protein during 96 h of reoxygenation after hypoxia and increasing MN/CA IX protein levels under permanent normoxia (n=3-4, means±SEM).

FIG. 12 shows the association of MN/CA IX protein levels after 24 h of hypoxia (0.1% O₂) with modified oxygen enhancement ratio (OER′), a measure of hypoxic radiation resistance obtained from clonogenic survival curves of cells irradiated at different O₂ concentrations, for HT 1080 human fibrosarcoma and FaDu human pharyngeal carcinoma cells [Vordermark et al., Int. J. Radiat. Oncol. Biol. Phys., 58: 1242-1250 (2004)]. Each data point represents one O₂ concentration (air, 5%, 1%, 0.1%). A correlation of hypoxic radiation resistance and increasing MN/CA IX protein was seen only in FaDu but not in HT 1080, resulting from the similar MN/CA IX curves at different O₂ concentrations shown in FIG. 10A for HT 1080.

FIG. 13A-B describes the impact of the availability of glucose (5.5 mM vs. no glucose), serum (10% fetal calf serum vs. no serum) and pH (6.7 vs. 7.4) on aerobic and hypoxic (24 h, 0.1% O₂) MN/CA IX protein levels in HT 1080 human fibrosarcoma and FaDu human pharyngeal carcinoma cells, using Western blot analysis with β-actin as loading control. Quantitative analysis indicates a requirement of glucose for hypoxic accumulation of MN/CA IX protein in HT 1080 and of both serum and glucose in FaDu. Modification of pH was only tested in fully supplemented medium and had a minor effect (n=3, means±SEM).

FIG. 14A-D illustrates the role of cell density for normoxic and hypoxic MN/CA IX and hypoxia inducible factor-1α (HIF-1α) protein accumulation. HT 1080 human fibrosarcoma and FaDu human pharyngeal carcinoma cells were plated at 200,000, 1,000,000 or 5,000,000 cells per 8-cm-diameter glass dish. Treatment with hypoxia (24 h, 0.1% O₂) or aerobic control conditions was initiated one day after plating. MN/CA IX protein was measured in whole-cell lysates or HIF-1α in nuclear extracts, using Western blot analysis (β-actin as loading control for CA IX, β-tubulin as loading control for HIF-1α). 14A-B: Quantitative analysis indicates normoxic induction of MN/CA IX at high cell density particularly in HT 1080. The loss of MN/CA IX protein after hypoxia in FaDu may be due to extremely dense culture conditions (n=3, means±SEM). 14C-D: Quantitative analysis showing a similar induction of HIF-1α in HT 1080 under dense-culture aerobic conditions as for MN/CA IX (FIG. 14A). In both cell lines, high density abolished hypoxic HIF-1α accumulation.

FIG. 15A-B shows representative MN/CA IX flow cytometry histograms of mixed cell suspensions with known percentage of aerobic and hypoxic (0.1% O₂, 24 h) HT 1080 human fibrosarcoma (A) and FaDu human pharyngeal carcinoma (B) cells. Unfixed cell suspensions were incubated with the anti-MN/CA IX antibody M75 and a FITC-conjugated secondary antibody. In HT 1080, the mean fluorescence intensity of MN/CA IX-negative and MN/CA IX-positive cells differed by a factor of about 70. In FaDu this factor was about 30 and apparently some hypoxic cells remained MN/CA IX-negative.

FIG. 16A-C describes the association of cellular MN/CA IX with cellular hypoxia and radiosensitivity. (A) Linear correlation of the known percentage of hypoxic cells and the percentage of MN/CA IX-positive cells measured by flow cytometry in mixed suspensions of normoxic and hypoxic (0.1% O₂, 24 h) HT 1080 human fibrosarcoma and FaDu human pharyngeal carcinoma cells, as shown in FIG. 15A-B (n=3, means±SEM). (B) Radiosensitivity of mixed suspensions with known percentages of normoxic and hypoxic (24 h, 0.1% O₂) HT 1080 human fibrosarcoma and FaDu human pharyngeal carcinoma cells. Cells were irradiated with 10 Gy under the respective conditions before mixing and surviving fraction determined by clonogenic survival assay. (C) Association of percentage of MN/CA IX-positive cells and surviving fraction after 10 Gy in the mixed cell suspensions with known percentages of hypoxic cells, as described in Example 7 below.

FIG. 17A-B shows the effect of different glucose concentrations under aerobic and hypoxic conditions (24 h, 0.1% O₂) on CA IX protein accumulation in (A) HT 1080 human fibrosarcoma or (B) FaDu human pharyngeal carcinoma cells (no serum, pH 7.4; representative Western blots).

### DETAILED DESCRIPTION

The following references provide updated information concerning the MN/CA9 gene and the MN/CA IX protein, which references are specifically incorporated by reference herein as well as references cited therein and are useful to clarify any inconsistent details concerning the MN gene and protein:
Barto\check{s}ová et al., “Expression of carbonic anhydrase IX in breast is associated with malignant tissues and is related to overexpression of c-erbB2,” Journal of Pathology, 197: 314-321 (2002)
Beasley et al., “Carbonic anhydrase IX, an endogenous hypoxia marker, expression in head and neck squamous cell carcinoma and its relationship to hypoxia, necrosis, and microvessel density,” Cancer Res., 61(13): 5262-5267 (Jul. 1, 2001)
Chia et al., “Prognostic Significance of a Novel Hypoxia Regulated Marker, Carbonic Anhydrase IX (MN/CA IX), in Invasive Breast Cancer,” Breast Cancer Research and Treatment, 64(1): pp. 43 (2000)
Chia et al., “Prognostic significance of a novel hypoxia-regulated marker, carbonic anhydrase IX, in invasive breast carcinoma,” J. Clin. Oncol., 19(16): 3660-3668 (Aug. 15, 2001)
Giatromanolaki et al., “Expression of Hypoxia-inducible Carbonic Anhydrase-9 Relates to Angiogenic Pathways and Independently to Poor Outcome in Non-Small Cell Lung Cancer,” Cancer Research, 61(21): 7992-7998 (Nov. 1, 2001)
Ivanov et al., “Expression of Hypoxia-Inducible Cell-Surface Transmembrane Carbonic Anhydrases in Human Cancer,” American Journal of Pathology, 158(3): 905-919 (March 2001)
Kaluz et al., “Transcriptional Regulation of the MN/CA9 Gene Coding for the Tumor-associated Carbonic Anhydrase IX,” The Journal of Biological Chemistry, 274(46): 32588-32595 (Nov. 12, 1999)
Kaluzova et al., “P53 tumour suppressor modulates transcription of the TATA-less gene coding for the tumour-associated carbonic anhydrase MN/CA IX in MaTu Cells,” Biochemica et Biophysica Acta, 1491: 20-26 (2000)
Kaluzova et al., “Characterization of the MN/CA9 promoter proximal region: a role for specificity protein (SP) and activator protein 1 (AP1) factors,” Biochemical Journal, 359(Pt 3): 669-677 (Nov. 1, 2001)
Kaluzova et al., “High cell density induces expression from the carbonic anhydrase 9 promoter,” Biotechniques, 36: 228-234 (2004)
Kivela et al., “Expression of transmembrane carbonic anhydrase isoenzymes IX and XII in normal human pancreas and pancreatic tumours,” Histochemistry and Cell Biology, 114(3): 197-204 (2000)
Koukourakis et al., “Hypoxia-regulated Carbonic Anhydrase-9 (CA9) Relates to Poor Vascularization and Resistance of Squamous Cell Head and Neck Cancer to Chemoradiotherapy,” Clinical Cancer Research, 7(11): 3399-3403 (November 2001)
Liao et al., “Identification of MN/CA (protein as a reliable diagnostic biomarker of clear cell carcinoma of the kidney,” Cancer Res., 57: 2827-2831 (1997)
Lieskovska et al., “Up-regulation of p53 by antisense expression of HPV18 E6 oncogene does not influence the level of MN/CA IX tumor-associated protein in HeLa cervical carcinoma cells,” International Journal of Oncology, 13: 1081-1086 (1998)
Lieskovska et al., “Study of in vitro conditions modulating expression of MN/CA IX protein in human cell lines derived from cervical carcinoma,” Neoplasma, 46:17-24 (1999)
Loncaster et al., “Carbonic Anhydrase (CAIX) Expression, a Potential New Intrinsic Marker of Hypoxia: Correlations with Tumor Oxygen Measurements and Prognosis in Locally Advanced Carcinoma of the Cervix,” Cancer Res, 61(17): 6394-6399 (Sep. 1, 2001)
Maseide et al., “Carbonic anhydrase IX as a marker for poor prognosis in soft tissue sarcoma,” Clin. Cancer Res., 10: 4464-4471 (2004)
Ortova Gut et al., “Gastric Hyperplasia in Mice With Targeted Disruption of the Carbonic Anhydrase Gene Car9,” Gastroenterology, 123: 1889-1903 (2002)
Parkkila et al., “Carbonic anhydrase inhibitor suppresses invasion of renal cancer cells in vitro,” PNAS (USA), 97(5): 2220-2224 (Feb. 29, 2000)
Pastorekova et al., “Carbonic anhydrase IX, a new player in a HIF-directed orchestra implicated in cell adhesion,” Abstract submitted to International Conference on Hypoxia/HIF Mediated Responses in Tumor Biology, Univ. of Manchester, United Kingdom (Nov. 27-29, 2002)
Saarnio et al., “Immunohistochemistry of Carbonic Anhydrase Isozyme IX (MN/CA IX) in Human Gut Reveals Polarized Expression in the Epithelial Cells with the Highest Proliferative Capacity,” Journal of Histochemistry & Cytochemistry, 46(4): 497-504 (1998)
Saarnio et al., “Immunohistochemical Study of Colorectal Tumors for Expression of a Novel Transmembrane Carbonic Anhydrase, MN/CA IX, with Potential Value as a Marker of Cell Proliferation,” Am. J. Pathol., 153(1): 279-285 (July 1998)
Stouracova et al., “Preliminary crystallographic study of an anti-MN/CA IX monoclonal antibody M75 Fab fragment complexed with its epitope peptide,” Abstract submitted to 20th European Crystallographic Meeting ECM 20 in Kraków (Aug. 25-31, 2001)
Turner et al., “The hypoxia-inducible genes VEGF and CA9 are differentially regulated in superficial vs invasive bladder cancer,” British Journal of Cancer, 86: 1276-1282 (2002)
Vermylen et al., “Carbonic anhydrase IX antigen differentiates between preneoplastic malignant lesions in non-small cell lung carcinoma,” Eur Respir J, 14: 806-811 (1999)
Wykoff et al., “Hypoxia-inducible Expression of Tumor-associated Carbonic Anhydrases,” Cancer Research, 60: 7075-7083 (Dec. 15, 2000)
Wykoff et al., “Expression of the hypoxia-inducible and tumor-associated carbonic anhydrases in ductal carcinoma in situ of the breast,” Am. J. Pathol., 158(3): 1011-1019 (March 2001)
Zavada et al., “Tumor-associated cell adhesion molecule MN/CA9: Identification of the binding site,” Cancer Detection and Prevention, 22 (Suppl. 1): 72 (Abstract #203) (1998)
Zavada et al., “Biological Activity of MN/CA IX Protein: Inhibition with Monoclonal Antibody or with Synthetic Oligopeptides,” Abstract submitted to European Association of Cancer Research Meeting in Halkidiki, Greece, May 30-Jun. 3, 2000
Zavada et al., “Human tumour-associated cell adhesion protein MN/CA IX: identification of M75 epitope and of the region mediating cell adhesion,” British Journal of Cancer, 82(11): 1808-1813 (2000)
Zavadova et al., “Two Functions of Tumor-Associated MN/CA IX Protein,” Abstract submitted to European Association of Cancer Research Meeting in Halkidiki, Greece, May 30-Jun. 3, 2000

### MN and Hypoxia

Particularly relied upon herein in regard to aspects of this invention that relate to MN and hypoxia, and MN/CA9's HRE are the following articles incorporated in U.S. Provisional Application 60/341,036 (filed Dec. 13, 2001), from which the instant application claims priority:
Wykoff et al., “Hypoxia-inducible Expression of Tumor-associated Carbonic Anhydrases,” Cancer Research, 60(24): 7075-7083 (Dec. 15, 2000)
Turner et al., “The hypoxia induced genes VEGF (vascular endothelial growth factor) and CA9 (carbonic anhydrase 9) are differentially regulated in superficial vs invasive human bladder cancer,” European Urology, 39(Supp. 5): pp. 171 (March 2001)
Wykoff et al., “Expression of the Hypoxia-Inducible and Tumor-Associated Carbonic Anhydrases in Ductal Carcinoma in Situ of the Breast,” American Journal of Pathology, 158(3): 1011-1019 (March 2001)
Beasley et al., “Carbonic Anhydrase IX, an Endogenous Hypoxia Marker, Expression in Head and Neck Squamous Cell Carcinoma and its Relationship to Hypoxia, Necrosis, and Microvessel Density,” Cancer Research, 61(13): 5262-5267 (Jul. 1, 2001)
Harris, A. L., “Hypoxia regulated transcriptome: Implications for tumour angiogenesis and therapy,” British Journal of Cancer, 85(Supp. 1): pp. 4 (July 2001)
Chia et al., “Prognostic Significance of a Novel Hypoxia-Regulated Marker, Carbonic Anhydrase IX, in Invasive Breast Carcinoma,” Journal of Clinical Oncology, 19(16): 3660-3668 (Aug. 15, 2001)
Loncaster et al., “Carbonic Anhydrase (CA IX) Expression, a Potential New Intrinsic Marker of Hypoxia: Correlations with Tumor Oxygen Measurements and Prognosis in Locally Advanced Carcinoma of the Cervix,” Cancer Research, 61(17): 6394-6399 (Sep. 1, 2001)
Giatromanolaki et al., “Expression of Hypoxia-inducible Carbonic Anhydrase-9 Relates to Angiogenic Pathways and Independently to Poor Outcome in Non-Small Cell Lung Cancer,” Cancer Research, 61(21): 7992-7998 (Nov. 1, 2001)
Koukourakis et al., “Hypoxia-regulated Carbonic Anhydrase-9 (CA9) Relates to Poor Vascularization and Resistance of Squamous Cell Head and Neck Cancer to Chemoradiotherapy,” Clinical Cancer Research, 7(11): 3399-3403 (November 2001)
O'Byrne et al., “Towards a biological staging model for operable non-small cell lung cancer,” Lung Cancer, 34(Supp. 2): S83-S89 (December 2001)

Further references concerning MN and hypoxia, or hypoxia more generally and/or radiobiology include the following:
Airley et al., “GLUT-1 and CA IX as intrinsic markers of hypoxia in carcinoma of the cervix: relationship to pimonidazole binding,” Int. J. Cancer, 104: 85-91 (2003)
Chrastina A., “High cell density-mediated pericellular hypoxia is a crucial factor inducing expression of the intrinsic hypoxia marker CA IX in vitro in HeLa cells,” Neoplasma, 50: 251-256 (2003)
Denekamp and Dasu, “Inducible repair and the two forms of tumour hypoxia—time for a paradigm shift,” Acta. Oncol., 38: 903-918 (1999)
Fyles et al., “Tumor hypoxia has independent predictor impact only in patients with node-negative cervix cancer,” J. Clin. Oncol., 20: 680-687 (2002).
Gross et al., “Calibration of misonidazole labeling by simultaneous measurement of oxygen tension and labeling density in multicellular spheroids,” Int. J. Cancer, 61: 567-573 (1995)
Hedley et al., “Carbonic anhydrase IX expression, hypoxia, and prognosis in patients with uterine cervical carcinomas,” Clin. Cancer Res., 9: 5666-5674 (2003)
Hui et al., “Coexpression of hypoxia-inducible factors 1alpha and 2alpha, carbonic anhydrase IX, and vascular endothelial growth factor in nasopharyngeal carcinoma and relationship to survival,” Clin. Cancer Res., 8: 2595-2604 (2002)
Jewell et al., “Induction of HIF-1 alpha in response to hypoxia is instantaneous,” FASEB J., 15: 1312-1314 (2001)
Kaanders et al., “Pimonidazole binding and tumor vascularity predict for treatment outcome in head and neck cancer,” Cancer Res., 62: 7066-7074 (2002)
Kaluz et al., “Lowered oxygen tension induces expression of the hypoxia marker MN/carbonic anhydrase IX in the absence of hypoxia-inducible factor 1 alpha stabilization: a role for phosphatidylinositol 3′-kinase,” Cancer Res., 62: 4469-4477 (2002)
Lal et al., “Transcriptional response to hypoxia in human tumors,” J Natl. Cancer Inst., 93: 1337-1343 (2001)
Nordsmark and Overgaard, “A confirmatory prognostic study on oxygenation status and loco-regional control in advanced head and neck squamous cell carcinoma treated by radiation therapy,” Radiother. Oncol., 57: 39-43 (2000)
Nordsmark et al., “Hypoxia in human soft tissue sarcomas: adverse impact on survival and no association with p53 mutations,” Br. J. Cancer, 84: 1070-1075 (2001)
Olive et al., “Carbonic anhydrase 9 as an endogenous marker for hypoxic cells in cervical cancer,” Cancer Res. 61: 8924-8929 (2001)
Rafajova et al., “Induction by hypoxia combined with low glucose or low bicarbonate and high posttranslational stability upon reoxygenation contribute to carbonic anhydrase IX expression in cancer cells,” Int. J. Oncol., 24: 995-1004 (2004)
Steel G. G., Basic clinical radiobiology, 2nd ed., London: Arnold (1997)
Swinson et al., “Carbonic anhydrase IX expression, a novel surrogate marker of tumor hypoxia, is associated with a poor prognosis in non-small-cell lung cancer,” J. Clin. Oncol., 21: 473-482 (2003)
Vordermark and Brown, “Evaluation of hypoxia-inducible factor-1α (HIF-1α) as an intrinsic marker of tumor hypoxia in U87 MG human glioblastoma: in-vitro and xenograft studies,” Int. J. Radiat. Oncol. Biol. Phys., 56: 1184-1193 (2003)
Vordermark and Brown, “Endogenous markers of tumor hypoxia: predictors of clinical radiation resistance?,” Strahlenther Onkol., 179: 8018-8011 (2003)
Vordermark et al., “Cell-type specific association of hypoxia-inducible factor-1α (HIF-1α) protein accumulation and radiobiologic tumor hypoxia,” Int. J. Radiat. Oncol. Biol. Phys. 58: 1242-1250 (2004)
Vordermark et al., “Green fluorescent protein is a suitable reporter of tumor hypoxia despite an oxygen requirement for chromophore formation,” Neoplasia, 3: 527-534 (2001)
Vordermark et al., “Similar radiation sensitivities of acutely and chronically hypoxic cells in HT 1080 fibrosarcoma xenografts,” Radiat Res, 159: 94-101 (2003)
   The above-listed articles and the references cited therein are hereby incorporated by reference

Studies of the MN/CA9 promoter demonstrated that the hypoxia-inducible response is mediated by HIF, and that it is dependent on a consensus HRE or consensus HBS (depending upon the terminology applied) lying adjacent to the initiation site. Studies of the MN/CA9 promoter also demonstrated that promoter fragments close to the transcription initiation site were sufficient to convey a hypoxia-inducible response.

The MN/CA9 promoter contains neither a TATA box nor a consensus initiator sequence at the cap site. The association of that unusual anatomy with tight regulation by hypoxia renders MN/CA9 of particular clinical interest. Also unusual and of particular clinical interest is the strong hypoxia-inducibility conveyed by the minimal MN/CA9 promoter (−36/+14) [SEQ ID NO: 106] and its putative HRE [SEQ ID NO: 105] or HBS [alternatively, SEQ ID NOS: 105, 107, 108, 109 or 110, most preferably SEQ ID NO: 105]. The MN/CA9 promoter HRE or HBS comprised in various MN/CA9 promoter fragments may be of considerable utility in the refinement of gene therapy vectors seeking to target therapeutic gene expression to hypoxic regions of tumors. (Wykoff et al. 2000).

Further refinement can be envisioned by one of skill in the art by positioning enhancer elements strategically within a MN/CA9 promoter fragment comprising a HRE/HBS. Still further refinement could be envisioned as placing the MN/CA9 HRE/HBS and associated flanking sequences within a promoter for another gene as considered to be strategically advantageous.

For example, Dachs et al. Nat. Med., 3: 515-520 (1997) describes in vitro experiments in which a PGK-1 HRE promoter is used to drive the expression of a bacterial cytosine deaminase gene, which gene product in turn activates the prodrug 5-fluorocytosine (5-FC) to 5-fluorouracil (5-FU). The overall effect is to sensitize human cells to the prodrug, to which they are normally resistant. A similar system could be applied to hypoxic cells, in order to selectively sensitize tumor hypoxic cells to a prodrug by transfecting them with an activating gene driven by an MN/CA9 HRE and promoter, followed by treatment with the prodrug. The advantage of using the MN/CA9 HRE and promoter, as opposed to other HIF-regulated genes, is that MN/CA9 expression correlates uniquely well with both tumor necrosis and low pO₂ tension.

Because of the unusually tight regulation of the MN/CA9 gene by hypoxia, the hypoxia-response element of the MN/CA9 gene is considered to be useful to determine HIF activation, either by microenvironmental hypoxia or genetic events such as VHL inactivation.

Although other hypoxia-induced proteins may be useful markers of hypoxia, MN/CA IX is induced at the same oxygen tension at which HIF-1α is induced and provides a measure of the percentage of the tumor population that is hypoxic (Beasley et al., 2001). MN/CA9 expression correlates with the oxygen diffusion distance and is expressed in a perinecrotic manner in head and neck squamous cell carcinoma (HNSCC).

To investigate the unusually tight regulation of MN/CA9 mRNA by hypoxia, the oxygen-dependent function of the MN/CA9 promoter was tested. Mutational analysis of the MN/CA9 hypoxia-response element sequence was performed in HeLa and CHO cell lines. Transient transfection experiments were performed using reporter plasmids containing full or partial sequences lying about 0.1 kb 5′ to the luciferase reporter gene. Mutations were made within the consensus HRE (or HBS) sequence to confirm the importance of the putative MN/CA9 HRE.

The invention provides in one aspect for the MN/CA9 HRE sequence to be used in a vector as described herein in the treatment of a patient with a hypoxia-related condition. Such vectors according to the invention can be administered by injection of the vector construct directly into a solid tumor, in the form of naked nucleic acid, preferably DNA, vectors. Alternatively, other vectors such as retroviruses may be used. According to the invention, the vector containing the MN/CA9 HRE sequence may be injected into the solid tumor, followed by administration of a prodrug in the case of a vector encoding a pro-drug activation system.

In one embodiment of the invention, the vector containing the MN/CA9 HRE sequence may be used in treatment of solid tumors. Alternatively, the vector containing the MN/CA9 HRE sequence may be used in treatment of other types of diseases where target cells are affected by hypoxia, such as acute and chronic vascular disease and pulmonary disease. For example, the gene regulated by the MN/CA9 HRE may encode for a cytokine or a growth factor. A vascular growth factor can be used to stimulate angiogenesis in hypoxic areas.

In another embodiment of the invention, the vector containing the MN/CA9 HRE sequence may be used to monitor or measure levels of hypoxia. Examples 1-4 below further elucidate the relationship between MN/CA9 and hypoxia, and aspects of this invention relating thereto.

### MN/CA IX Assays to Predict Tissue Radioresistance

It is one discovery of the invention that the level of MN/CA IX induction at different oxygen levels, and corresponding cellular radiation resistance, varies among cell types in vitro, and that therefore the utility of assays to predict the radioresistance of a tissue from MN/CA IX levels would preferably first be tested on a cell-by-cell basis. In 2004, one of the inventors reported a similar finding for a correlation between HIF-1α levels and radiation resistance [Vordermark et al., Int. J. Rad. Oncol. Biol. Phys., 58(4): 1242-1250 (2004)]. The conclusion was reached that the use of HIF-1α as a marker for radiobiologically relevant hypoxia is cell-type-specific. However, there are many reasons why MN/CA IX would be a better hypoxic marker than HIF-1α: most importantly, MN/CA IX is a more stable protein than HIF-1α. HIF-1α returns to basal levels within 15 minutes of reoxygenation, whereas MN/CA IX persists for at least 72 hours.

The invention provides methods to predict the degree of radioresistance of an affected tissue in a patient with a preneoplastic/neoplastic disease, particularly a disease associated with hypoxic tumors. The methods include quantifying the level and/or extent of MN/CA IX protein/polypeptide, if any, present in a sample taken from a patient that has been diagnosed with prenoplastic/neoplastic disease, and predicting the degree of radioresistance of the subject sample by extrapolating from the correlation of MN/CA IX levels and degree of radioresistance in a preliminary in vitro assay. The methods can be used, for example, to aid in the selection of therapies, specifically, to decide whether or not to use radiation therapy for a cancer patient.

In one aspect, the present invention is directed to a method to predict the degree of radioresistance of an affected tissue in a patient with a preneoplastic/neoplastic disease, using two assays: I.) a preliminary in vitro screening assay which correlates MN/CA IX levels with levels of hypoxia and degrees of radioresistance found in cells comparable to the subject patient tissue; and II.) a subsequent quantitative immunoassay of MN/CA IX levels in the patient tissue which is used to extrapolate a predicted radioresistance from the correlation found in the preliminary assay.

As used herein, “hypoxia” is defined on a tissue-by-tissue basis, as an O₂ level that is below the normal physiological O₂ levels found in a specific tissue.

As used herein, “degree of radioresistance” refers to the radioresistance of a tissue or cell type to killing by radiation, and not to a relative resistance of a patient to a cure using radiotherapy.

I. Preliminary In Vitro Screening Assay of Comparable Cells for Radiobiologically Relevant Tumor Hypoxia

One premise of the present invention is that before one can use immunoassays of MN/CA IX levels in a preneoplastic/neoplastic tissue to predict its radioresistance, preferably it would be established that the MN/CA IX levels found in that tissue correlate with, and vary within the ranges of “radiobiologically relevant tumor hypoxia.” As used herein, “radiobiologically relevant tumor hypoxia” is defined as levels of hypoxia that affect the radiation sensitivity of the subject cell type.

In other words, two requirements for a preneoplastic/neoplastic tissue which can be assayed according to the immunoassays of this aspect of the invention to predict tissue radioresistance from tissue MN/CA IX levels, are that 1) MN/CA IX protein is not already maximally expressed in the tissue at about 5% O₂; and 2) between cellular oxygen levels of about 5% and 0.1% O₂, changes in MN/CA IX protein induction levels would preferably correspond with changes in the oxygen levels. The “corresponding changes” in MN/CA IX levels may be inversely proportional to changes in O₂ levels, or the MN/CA IX levels may vary qualitatively with changes in O₂ levels, so long as the lower the O₂ concentration, the higher the MN/CA IX levels.

Such preliminary in vitro screening tests can be performed on biopsy preneoplastic/neoplastic tissues of the patient being considered for radiation therapy, or can be performed on comparable cells isolated from an organism of the same taxonomic classification as the subject vertebrate. One of skill in the art would be able to select comparable cells suitable for such in vitro tests, from the model cell lines that are typically used in assays relevant to individual preneoplastic/neoplastic diseases, particularly those diseases associated with hypoxic tumors. For example, in Examples 5-7, the FaDu human pharyngeal carcinoma cell line is used as a model for head and neck cancer.

It can be appreciated by ones of skill in the art that alternate methods, in addition to those disclosed herein, can be used in the in vitro prescreening assays of comparable cells. For example, different schedules of in vitro hypoxia, other manipulations of culture conditions, and alternative time points can be chosen for assaying MN/CA IX levels in the comparable cells under the various conditions of hypoxia. To determine maximal MN/CA IX expression in controls, treatments by 0.1% O₂ or DFO (hypoxia-mimetic chelating agent desferrioxamine), among other treatments, may be used. The detection and quantitation of MN/CA IX protein or MN/CA IX polypeptide in the comparable cells can be performed, for example, by Western blots, enzyme-linked immunosorbent assays, radioimmunoassays, competition immunoassays, dual antibody sandwich assays, immunohistochemical staining assays, agglutination assays, fluorescent immunoassays, immunoelectron and scanning microscopy using immunogold, among other assays commonly known in the art.

Alternative methods for measuring hypoxic radiation resistance in the comparable cells, in addition to those disclosed herein, may be used. For example, alternative modified oxygen enhancement ratios may be used, in which the D10 (radiation dose producing a surviving fraction of 10%) of the respective hypoxic condition may be divided by the D10 at 0.1% O₂, anoxia, or some other oxygen level in between. Alternative radiation doses (ranges, multiple doses, administration of doses), in addition to those disclosed herein, may also be used in the preliminary in vitro screening assays.

Exemplary In Vitro Prescreening Assays of Comparable Cells:

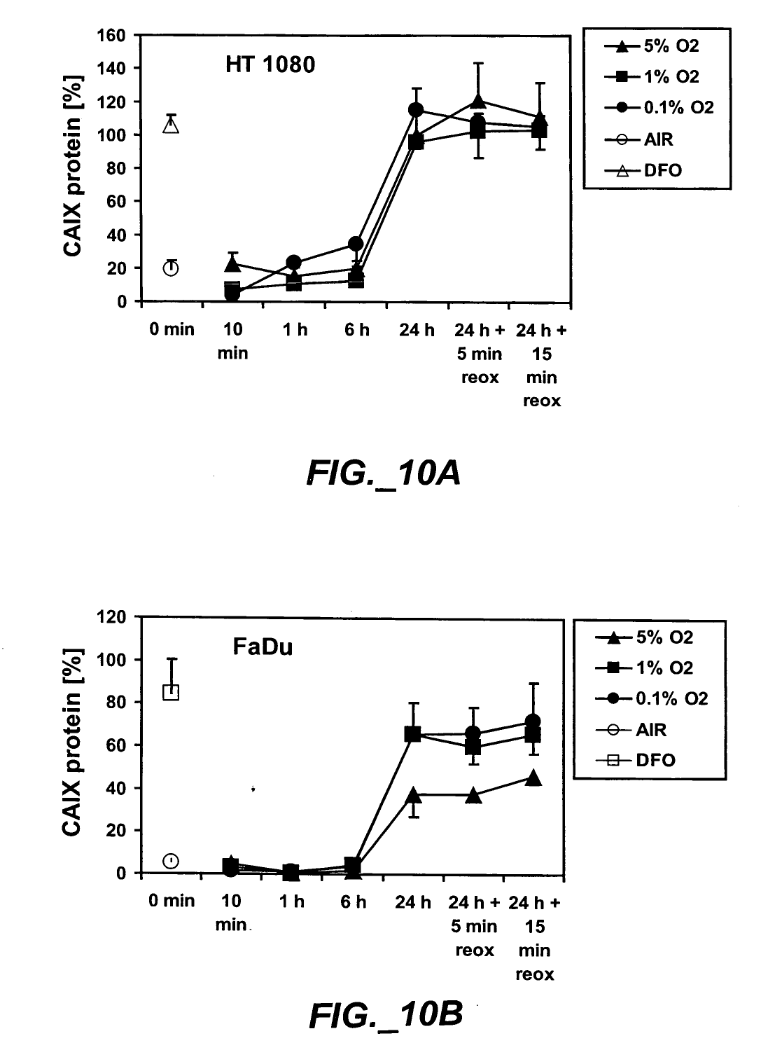

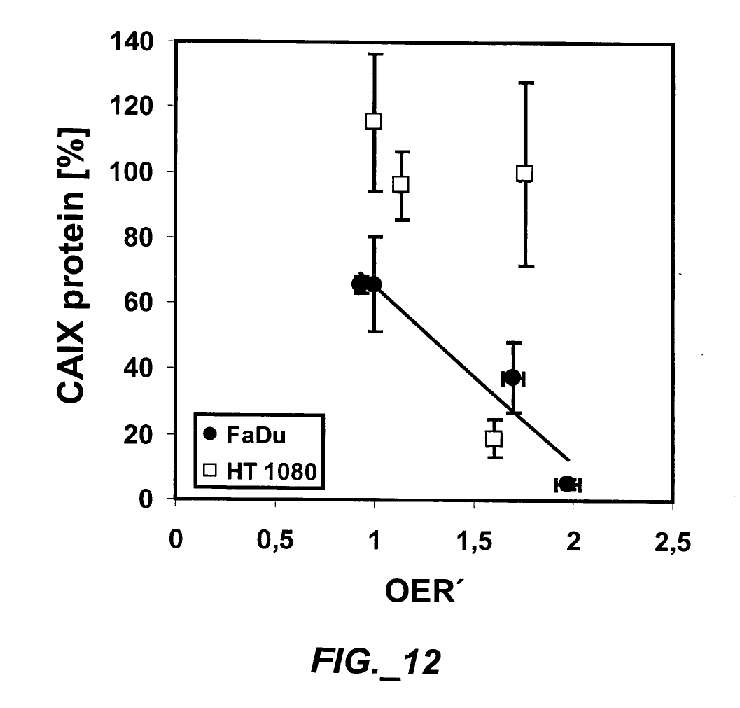

The results of exemplary in vitro prescreening assays of comparable model cells is shown in Example 5 and FIGS. 10-12 below. In the described assays, FaDu and HT 1080 cells were exposed to in vitro hypoxia at 5%, 1% and 0.1% O₂ and reoxygenated for various times, and the levels of MN/CA IX were quantitated by Western blots (using β-actin as loading control). As shown in FIG. 12, MN/CA IX levels after 24 hours of hypoxia were compared with the modified enhancement ratio (OER′), a measure of hypoxic radiation resistance, determined in the same cell lines at the same oxygen levels.

Of the two cell lines tested by such a preliminary in vitro screening assay, only the FaDu cell line met both of the above criteria. That is, MN/CA IX was submaximally expressed at 5% O₂, and changes in MN/CA IX protein induction levels corresponded with oxygen changes between 5% and 0.1% O₂. The marker protein MN/CA IX reached maximal levels of expression already at 5% O₂ in HT 1080, and only half-maximal levels in FaDu at 5%. Therefore, when these cells were later tested for a correlation of MN/CA IX and radioresistance, a reasonable association between the expression of MN/CA IX and hypoxic radioresistance was seen in FaDu but not in HT 1080. This result potentially limits the types of preneoplastic/neoplastic tissues that may be assayed according to the predictive immunoassays of the invention.

II. Immunoassays to Predict Radioresistance of a Preneoplastic/Neoplastic Tissue

Subsequent to the above in vitro cellular assay, the invention provides for quantitative immunoassays of patient samples that can be used to predict radioresistance for those preneoplastic/neoplastic tissues, wherein MN/CA IX protein/polypeptide tissue levels indicate radiobiologically relevant hypoxia. Such quantititative immunoassays comprise predicting the degree of radioresistance of the subject vertebrate tissue by comparing the MN/CA IX protein/polypeptide levels found in the subject tissue with the correlation of MN/CA IX levels with cellular radioresistance found in the comparable cells, and extrapolating therefrom a predicted degree of radioresistance of the subject vertebrate tissue.

Preneoplastic/Neoplastic Tissues

In a preferred embodiment of the invention, MN/CA IX protein/polypeptide is quantitated in a sample, from a subject vertebrate with a preneoplastic/neoplastic disease, preferably a disease associated with tumor hypoxia. Such samples can be tissue specimens, tissue extracts, body fluids, cells, cell lysates and cell extracts, among other samples. Preferred preneoplastic/neoplastic diseases are preneoplastic/neoplastic diseases of head and neck, mammary, urinary tract, kidney, bladder, ovarian, uterine, cervical, endometrial, vaginal, vulvar, prostate, liver, lung, skin, thyroid, pancreatic, testicular, brain, gastrointestinal, colon, colorectal and mesodermal tissues. Preferred tissue specimens to assay by immunohistochemical staining, for example, include cell smears, histological sections from biopsied tissues or organs, and imprint preparations among other tissue samples. An exemplary immunohistochemical staining protocol is described below in the Materials and Methods section.

Such tissue specimens can be variously maintained, for example, they can be fresh, frozen, or formalin-, alcohol- or acetone- or otherwise fixed and/or paraffin-embedded and deparaffinized. Biopsied tissue samples can be, for example, those samples removed by aspiration, bite, brush, cone, chorionic villus, endoscopic, excisional, incisional, needle, percutaneous punch, and surface biopsies, among other biopsy techniques. Preferred tissue samples are formalin-fixed, paraffin-embedded tissue samples.

It can be appreciated by those of skill in the art that various other preneoplastic/neoplastic samples can be used to quantify the MN/CA IX gene expression products. For example, the sample may be taken from a tumor or from a metastatic lesion derived from a tumor.

Detection and Quantification

A preferred method of quantifying the level of MN/CA IX protein/polypeptide in a patient sample is by immunohistochemical staining. More preferably, said MN/CA IX quantitating step comprises determining the percentage of immunoreactive cells and/or the intensity or extent of immunostaining of immunoreactive cells. Still more preferably, said MN/CA IX quantitating step comprises the use of the M75 monoclonal antibody secreted by the hybridoma VU-M75 which has been deposited under the Budapest Treaty at the American Type Culture Collection under Accession No. ATCC HB 11128.

Many formats can be adapted for use with the methods of the present invention. The detection and quantitation of MN/CA IX protein or MN/CA IX polypeptide can be performed, for example, by Western blots, enzyme-linked immunosorbent assays, radioimmunoassays, competition immunoassays, dual antibody sandwich assays, immunohistochemical staining assays, agglutination assays, fluorescent immunoassays, immunoelectron and scanning microscopy using immunogold, among other assays commonly known in the art. The quantitation of MN/CA9 gene expression products in such assays can be adapted by conventional methods known in the art; for example, if the detection method is by immunohistochemical staining, the quantitation of MN/CA IX protein or MN/CA IX polypeptide can be performed by determining the percentage of immunoreactive cells and/or the intensity or extent of immunostaining of immunoreactive cells, and can additionally comprise addition or multiplication of these values, or other mathematical calculations using these values.

It is also apparent to one skilled in the art of immunoassays that MN/CA IX proteins or polypeptides can be used to detect and quantitate MN/CA IX antigen in body tissues and/or cells of patients. In one such embodiment, an immunometric assay may be used in which a labelled antibody made to MN/CA IX protein is used. In such an assay, the amount of labelled antibody which complexes with the antigen-bound antibody is directly proportional to the amount of MN/CA IX antigen in the sample.

### MN Gene and Protein

MN/CA IX was first identified in HeLa cells, derived from human carcinoma of cervix uteri, as both a plasma membrane and nuclear protein with an apparent molecular weight of 58 and 54 kilodaltons (kDa) as estimated by Western blotting. It is N-glycosylated with a single 3 kDa carbohydrate chain and under non-reducing conditions forms S-S-linked oligomers [Pastorekova et al., Virology, 187: 620-626 (1992); Pastorek et al., Oncogene, 9: 2788-2888 (1994)]. MN/CA IX is a transmembrane protein located at the cell surface, although in some cases it has been detected in the nucleus [Zavada et al., Int. J. Cancer, 54: 268-274 (1993); Pastorekova et al., supra].

MN is manifested in HeLa cells by a twin protein, p54/58N. Immunoblots using a monoclonal antibody reactive with p54/58N (MAb M75) revealed two bands at 54 kd and 58 kd. Those two bands may correspond to one type of protein that most probably differs by post-translational processing. Herein, the phrase “twin protein” indicates p54/58N.

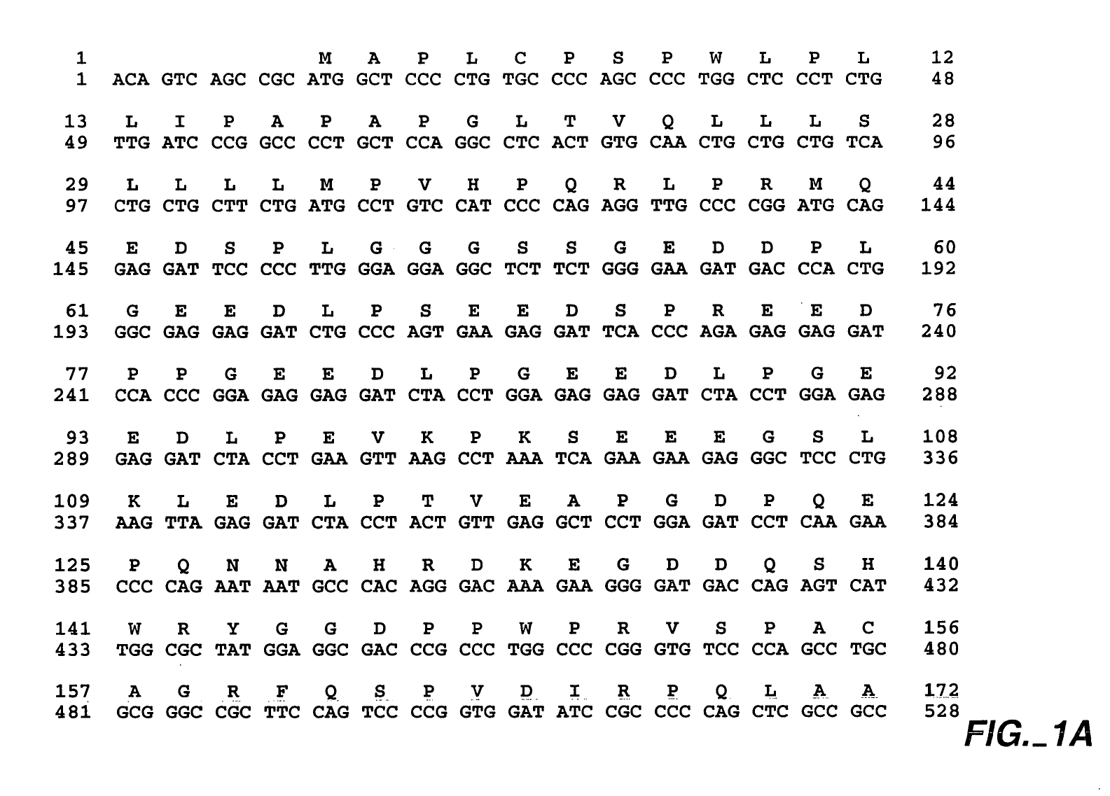

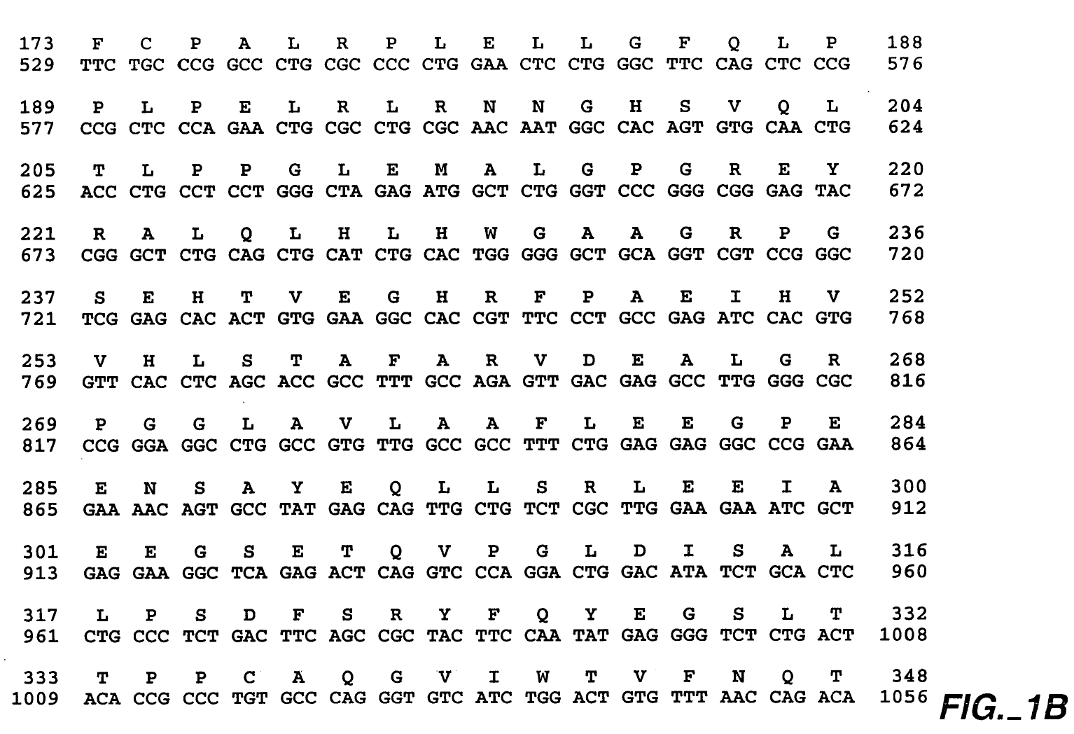

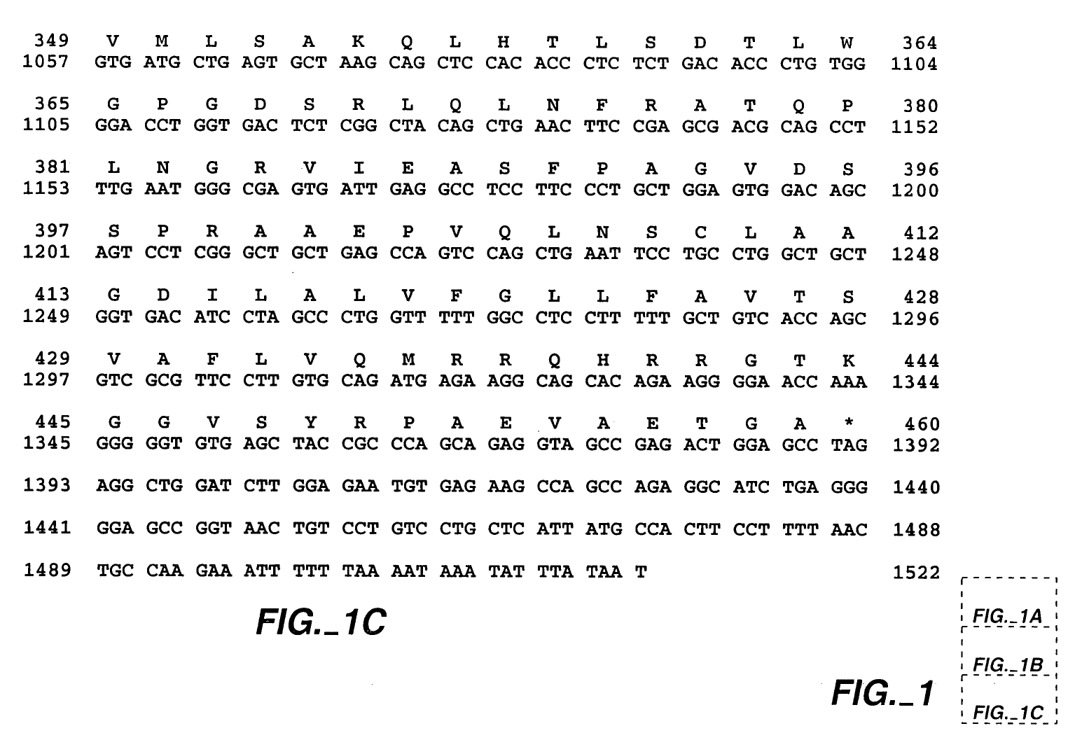

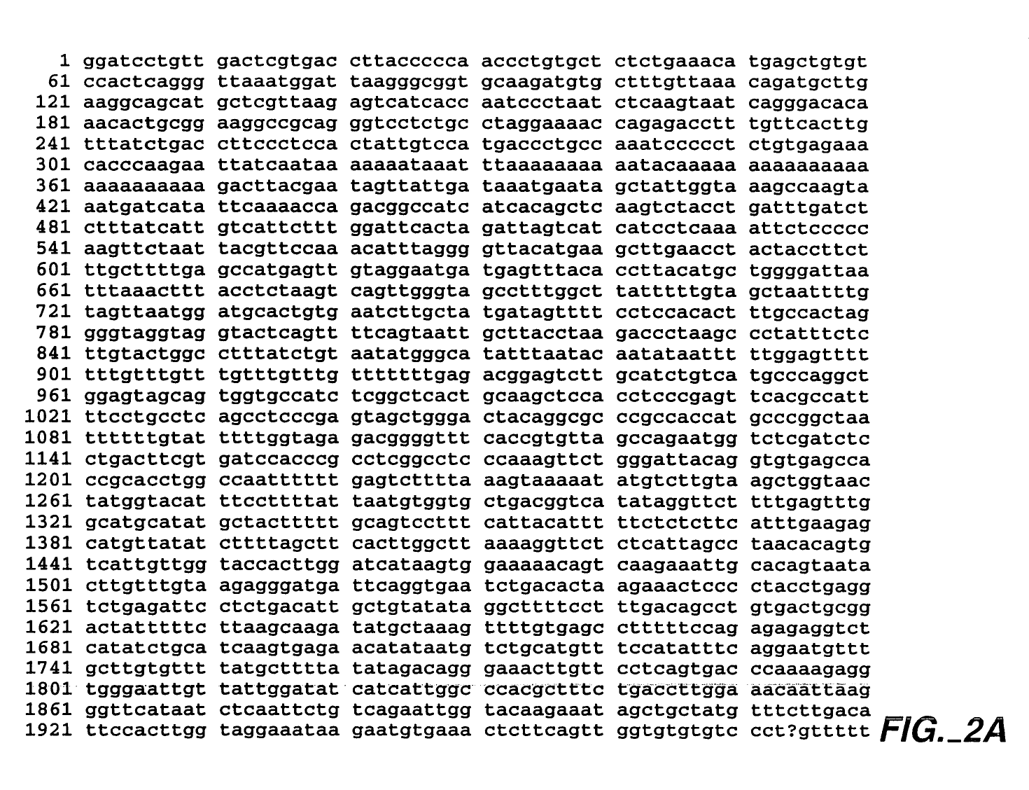

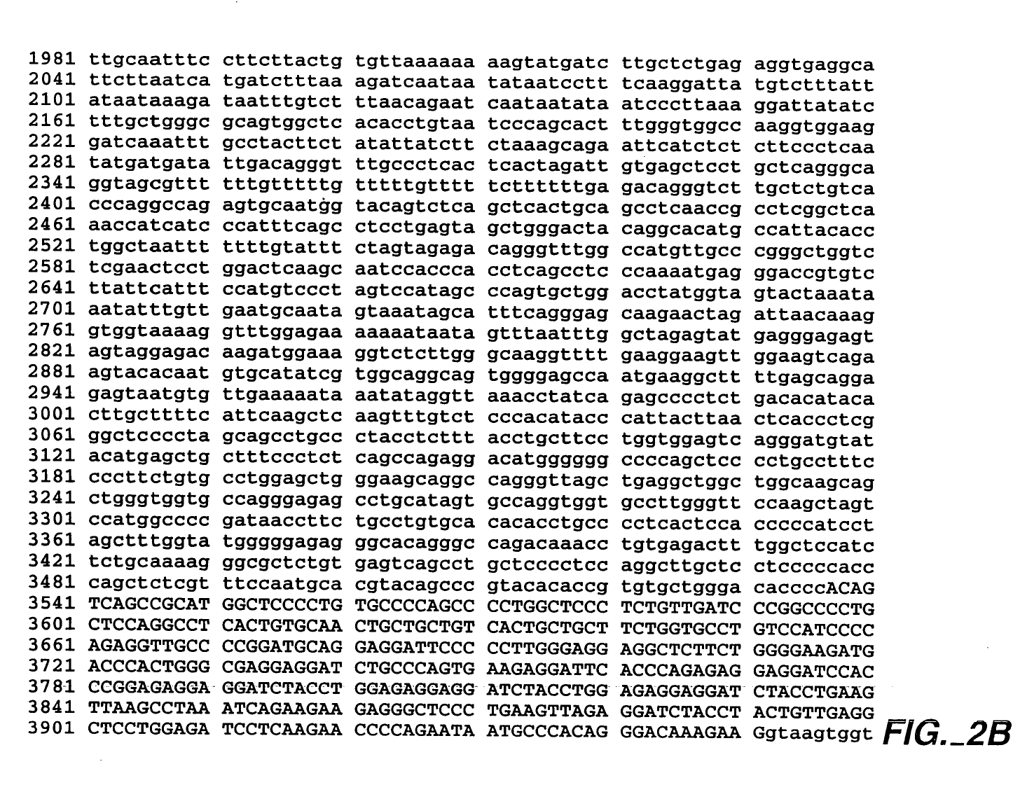

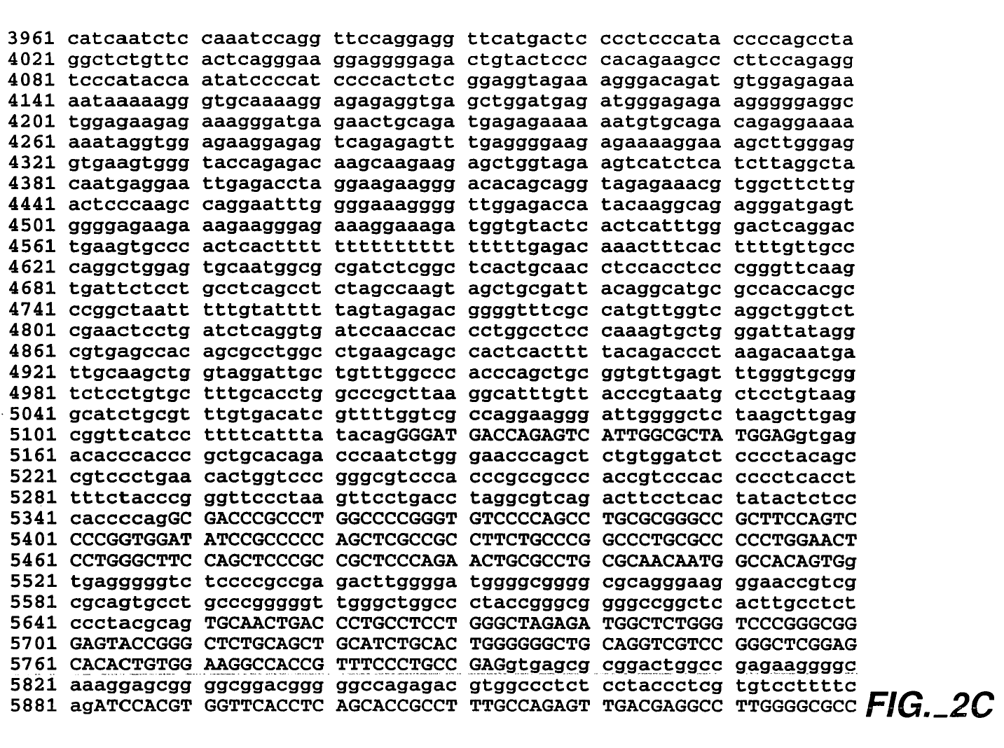

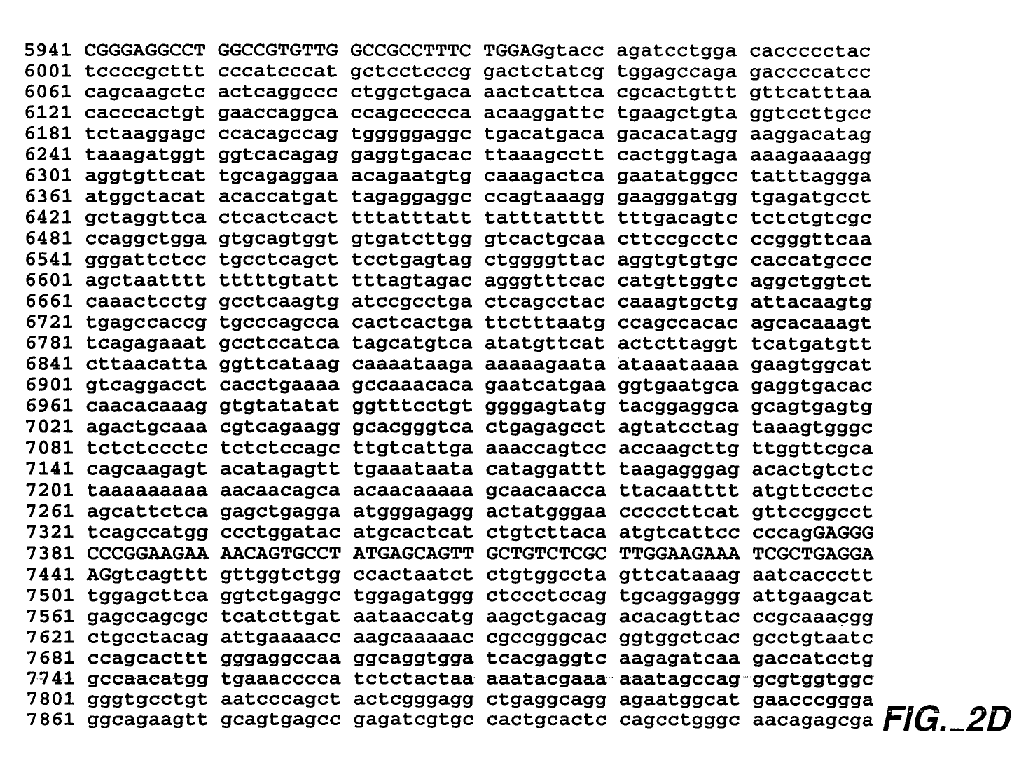

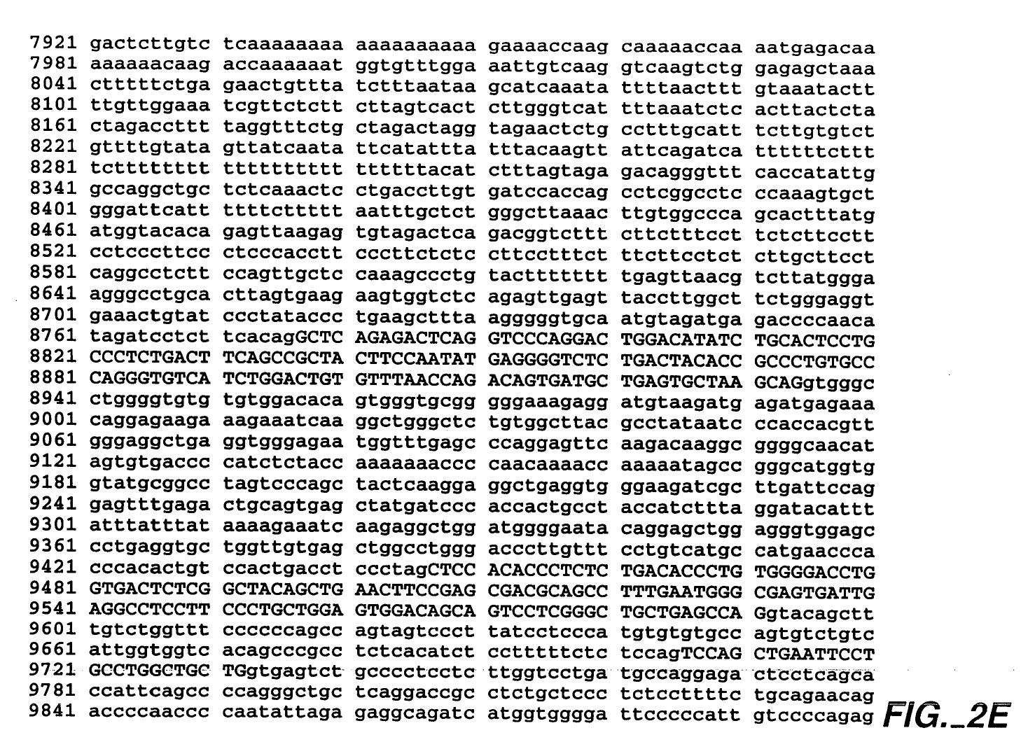

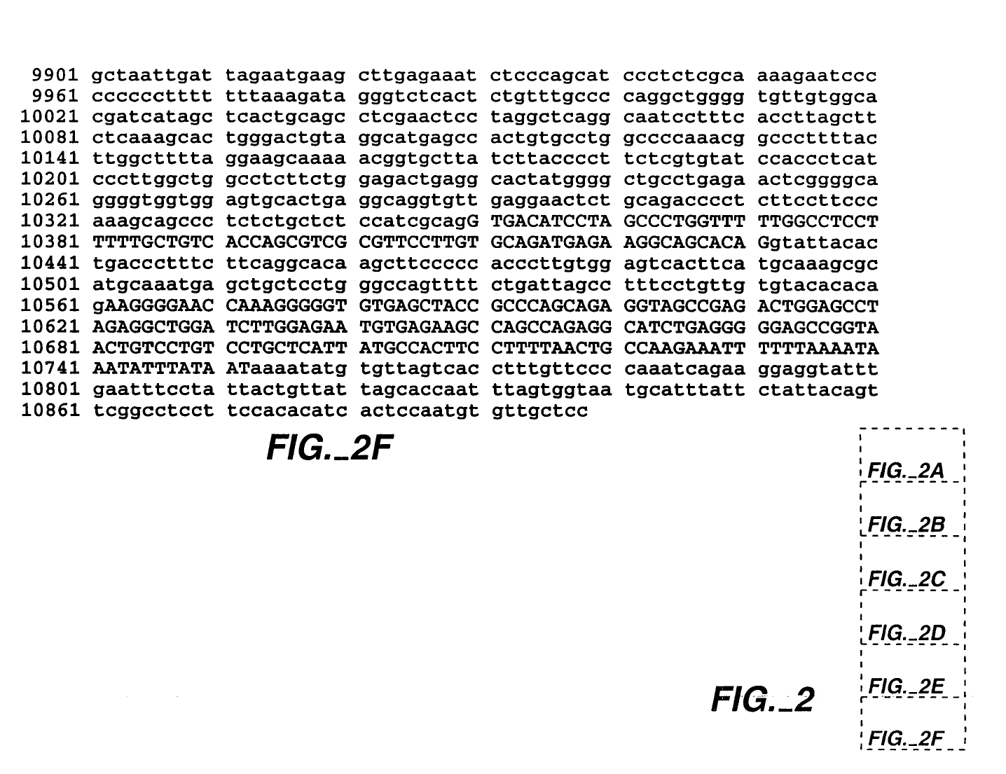

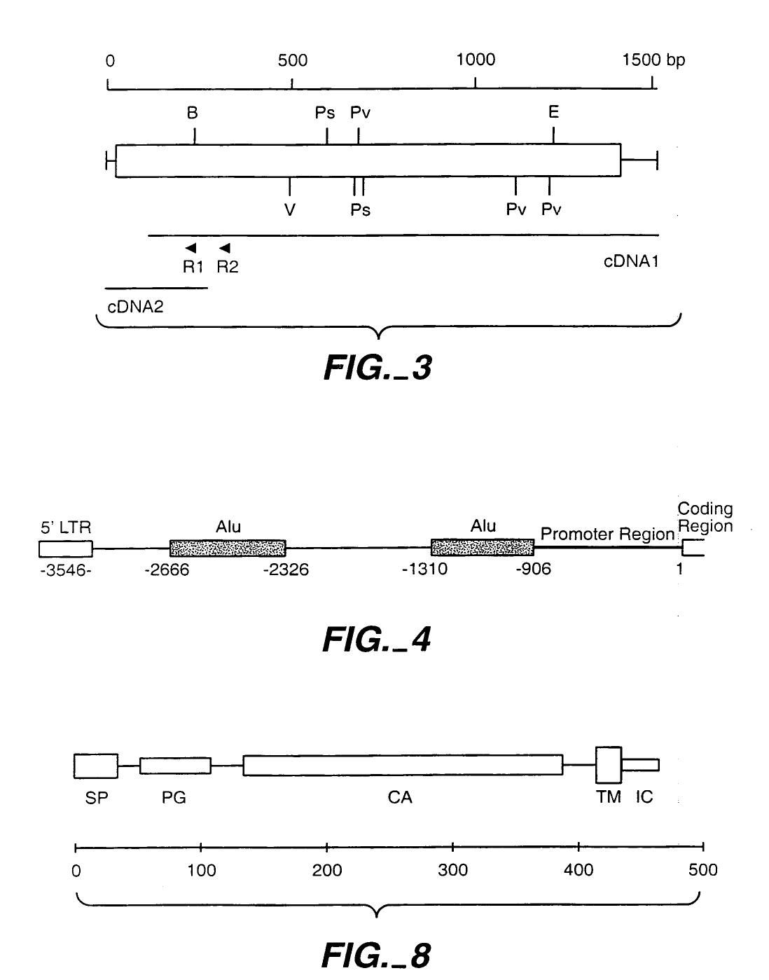

Zavada et al., WO 93/18152 and/or WO 95/34650 disclose the MN cDNA sequence (SEQ ID NO: 1) shown herein in FIG. 1A-1C, the MN amino acid sequence (SEQ ID NO: 2) also shown in FIG. 1A-1C, and the MN genomic sequence (SEQ ID NO: 5) shown herein in FIG. 2A-2F. The MN gene is organized into 11 exons and 10 introns.

The first thirty-seven amino acids of the MN protein shown in FIG. 1A-1C is the putative MN signal peptide [SEQ ID NO: 6]. The MN protein has an extracellular domain [amino acids (aa) 38-414 of FIG. 1A-1C (SEQ ID NO: 82)], a transmembrane domain [aa 415-434 (SEQ ID NO: 47)] and an intracellular domain [aa 435-459 (SEQ ID NO: 48)]. The extracellular domain contains the proteoglycan-like domain [aa 53-111 (SEQ ID NO: 45)] and the carbonic anhydrase (CA) domain [aa 135-391 (SEQ ID NO: 46)].

MN protein is considered to be a uniquely suitable target for cancer therapy for a number of reasons including the following. (1) It is localized on the cell surface, rendering it accessible. (2) It is expressed in a high percentage of human carcinomas (e.g., uterine cervical, renal, colon, breast, esophageal, lung, head and neck carcinomas, among others), but is not normally expressed to any significant extent in the normal tissues from which such carcinomas originate. (3) It is normally expressed only in the stomach mucosa and in some epithelia of the digestive tract (epithelium of gallbladder and small intestine). An anatomic barrier thereby exists between the MN-expressing preneoplastic/neoplastic and MN-expressing normal tissues. Drugs, including antibodies, can thus be administered which can reach tumors without interfering with MN-expressing normal tissues. (4) MAb M75 has a high affinity and specificity to MN protein. (5) MN cDNA and MN genomic clones which encompass the protein-coding and gene regulatory sequences have been isolated. (6) MN-specific antibodies have been shown to have among the highest tumor uptakes reported in clinical studies with antitumor antibodies in solid tumors, as shown for the MN-specific chimeric antibody G250 in animal studies and in Phase I clinical trials with renal carcinoma patients. [Steffens et al., J. Clin. Oncol. 15: 1529 (1997).] Also, MN-specific antibodies have low uptake in normal tissues.

### MN Gene—Cloning and Sequencing

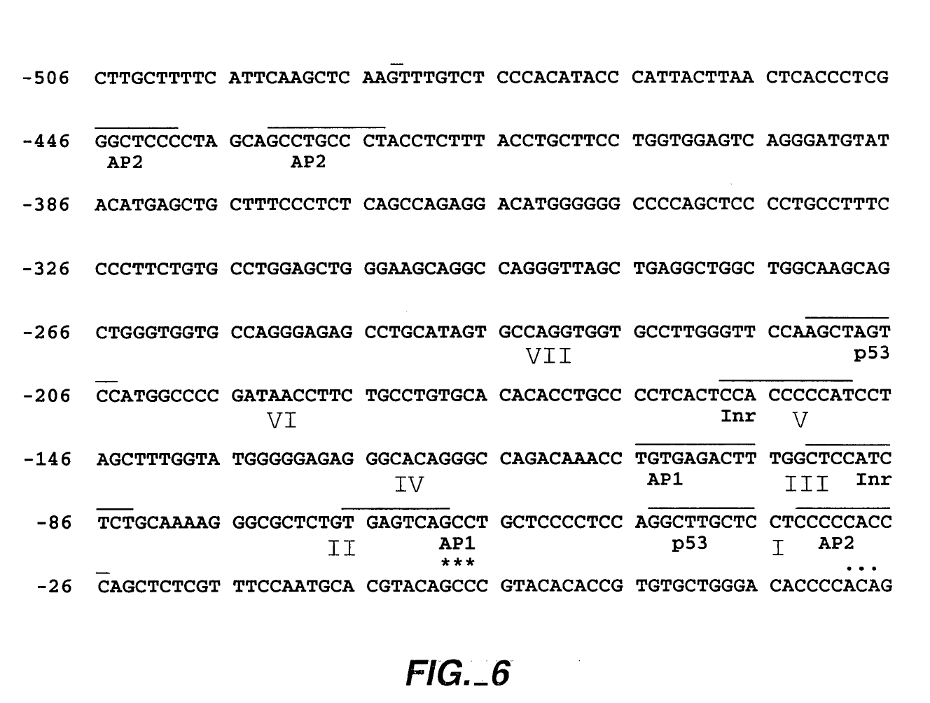

FIG. 1A-C provides the nucleotide sequence for a full-length MN cDNA clone isolated as described below [SEQ ID NO: 1]. FIG. 2A-F provides a complete MN genomic sequence [SEQ ID NO: 5]. FIG. 6 shows the nucleotide sequence for a proposed MN promoter [SEQ ID NO: 22].

It is understood that because of the degeneracy of the genetic code, that is, that more than one codon will code for one amino acid [for example, the codons TTA, TTG, CTT, CTC, CTA and CTG each code for the amino acid leucine (leu)], that variations of the nucleotide sequences in, for example, SEQ ID NOS: 1 and 5 wherein one codon is substituted for another, would produce a substantially equivalent protein or polypeptide according to this invention. All such variations in the nucleotide sequences of the MN cDNA and complementary nucleic acid sequences are included within the scope of this invention.

It is further understood that the nucleotide sequences herein described and shown in FIGS. 1, 2 and 6, represent only the precise structures of the cDNA, genomic and promoter nucleotide sequences isolated and described herein. It is expected that slightly modified nucleotide sequences will be found or can be modified by techniques known in the art to code for substantially similar or homologous MN proteins and polypeptides, for example, those having similar epitopes, and such nucleotide sequences and proteins/polypeptides are considered to be equivalents for the purpose of this invention. DNA or RNA having equivalent codons is considered within the scope of the invention, as are synthetic nucleic acid sequences that encode proteins/polypeptides homologous or substantially homologous to MN proteins/polypeptides, as well as those nucleic acid sequences that would hybridize to said exemplary sequences [SEQ. ID. NOS. 1, 5 and 22] under stringent conditions, or that, but for the degeneracy of the genetic code would hybridize to said cDNA nucleotide sequences under stringent hybridization conditions. Modifications and variations of nucleic acid sequences as indicated herein are considered to result in sequences that are substantially the same as the exemplary MN sequences and fragments thereof.

Stringent hybridization conditions are considered herein to conform to standard hybridization conditions understood in the art to be stringent. For example, it is generally understood that stringent conditions encompass relatively low salt and/or high temperature conditions, such as provided by 0.02 M to 0.15 M NaCl at temperatures of 50° C. to 70° C. Less stringent conditions, such as, 0.15 M to 0.9 M salt at temperatures ranging from 20° C. to 55° C. can be made more stringent by adding increasing amounts of formamide, which serves to destabilize hybrid duplexes as does increased temperature.

Exemplary stringent hybridization conditions are described in Sambrook et al., Molecular Cloning: A Laboratory Manual, pages 1.91 and 9.47-9.51 (Second Edition, Cold Spring Harbor Laboratory Press; Cold Spring Harbor, N.Y.; 1989); Maniatis et al., Molecular Cloning: A Laboratory Manual, pages 387-389 (Cold Spring Harbor Laboratory; Cold Spring Harbor, N.Y.; 1982); Tsuchiya et al., Oral Surgery, Oral Medicine, Oral Pathology, 71(6): 721-725 (June 1991).

Zavada et al., WO 95/34650 described how a partial MN cDNA clone, a full-length MN cDNA clone and MN genomic clones were isolated and sequenced. Also, Zavada et al., Int. J. Cancer, 54: 268 (1993) describes the isolation and sequencing of a partial MN cDNA of 1397 bp in length. Briefly, attempts to isolate a full-length clone from the original cDNA library failed. Therefore, the inventors performed a rapid amplification of cDNA ends (RACE) using MN-specific primers, R1 and R2 [SEQ ID NOS: 7 and 8], derived from the 5′ region of the original cDNA clone. The RACE product was inserted into pBluescript, and the entire population of recombinant plasmids was sequenced with an MN-specific primer ODN1 [SEQ ID NO: 3]. In that way, a reliable sequence at the very 5′ end of the MN cDNA as shown in FIG. 1 [SEQ ID NO: 1] was obtained.

Specifically, RACE was performed using 5′ RACE System [GIBCO BRL; Gaithersburg, Md. (USA)] as follows. 1 μg of mRNA (the same as above) was used as a template for the first strand cDNA synthesis which was primed by the MN-specific antisense oligonucleotide, R1 (5′-TGGGGTTCTTGAGGATCTCCAGGAG-3′) [SEQ ID NO: 7]. The first strand product was precipitated twice in the presence of ammonium acetate and a homopolymeric C tail was attached to its 3′ end by TdT. Tailed cDNA was then amplified by PCR using a nested primer, R2 (5′-CTCTAACTTCAGGGAGCCCTCTTCTT-3′) [SEQ ID NO: 8] and an anchor primer that anneals to the homopolymeric tail (5′-CUACUACUACUAGGCCACGCGTCGAC TAGTACGGGI IGGGIIGGGIIG-3′) [SEQ ID NO: 9]. The amplified product was digested with BamHI and SalI restriction enzymes and cloned into pBluescript II KS plasmid. After transformation, plasmid DNA was purified from the whole population of transformed cells and used as a template for sequencing with the MN-specific primer ODN1 [SEQ ID NO: 3; a 29-mer 5′ CGCCCAGTGGGTCATCTTCCCCAGAAGAG 3′].

To study MN regulation, MN genomic clones were isolated. One MN genomic clone (Bd3) was isolated from a human cosmid library prepared from fetal brain using both MN cDNA as a probe and the MN-specific primers derived from the 5′ end of the cDNA ODN1 [SEQ ID NO: 3, supra] and ODN2 [SEQ. ID NO.: 4; 19-mer (5′ GGAATCCTCCTGCATCCGG 3′)]. Sequence analysis revealed that that genomic clone covered a region upstream from a MN transcription start site and ending with the BamHI restriction site localized inside the MN cDNA. Other MN genomic clones can be similarly isolated.

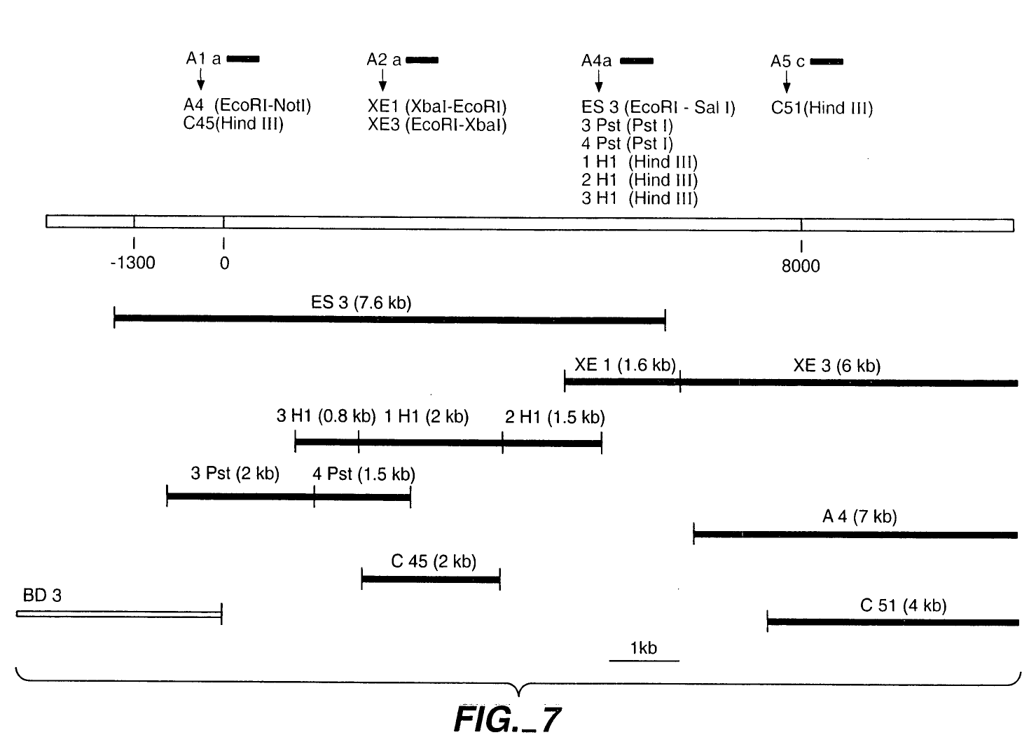

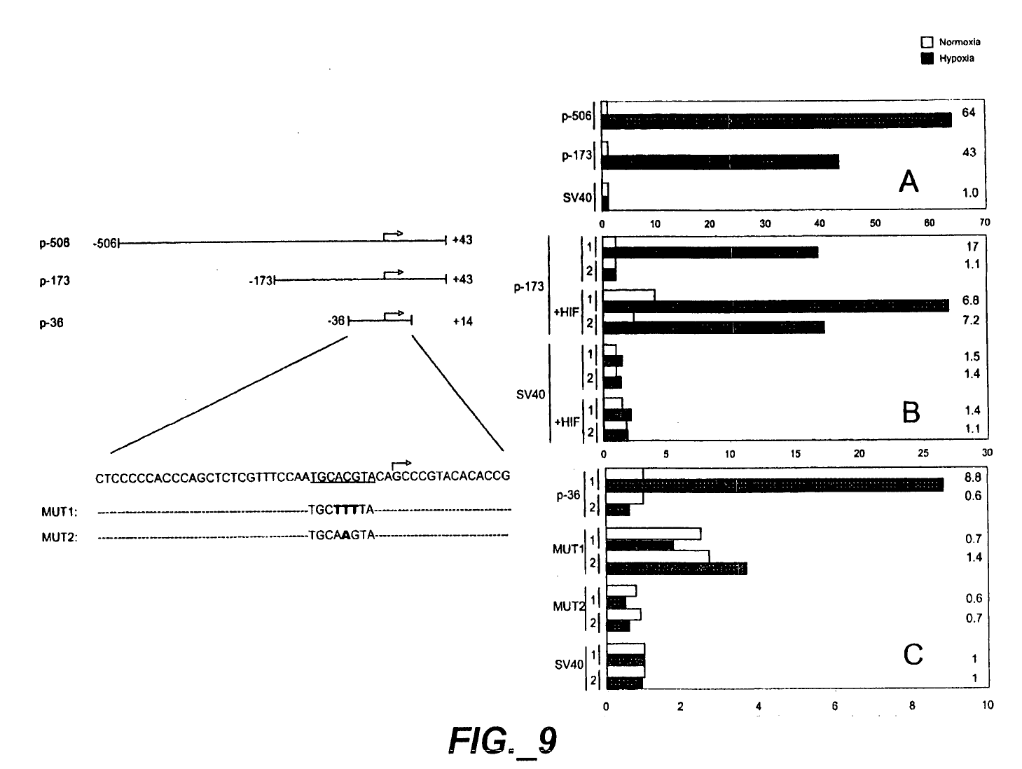

FIG. 7 provides a schematic of the alignment of MN genomic clones according to the transcription initiation site. Plasmids containing the A4a clone and the XE1 and XE3 subclones were deposited at the American Type Culture Collection (ATCC) on Jun. 6, 1995, respectively under ATCC Deposit Nos. 97199, 97200, and 97198.

Exon-Intron Structure of Complete MN Genomic Region

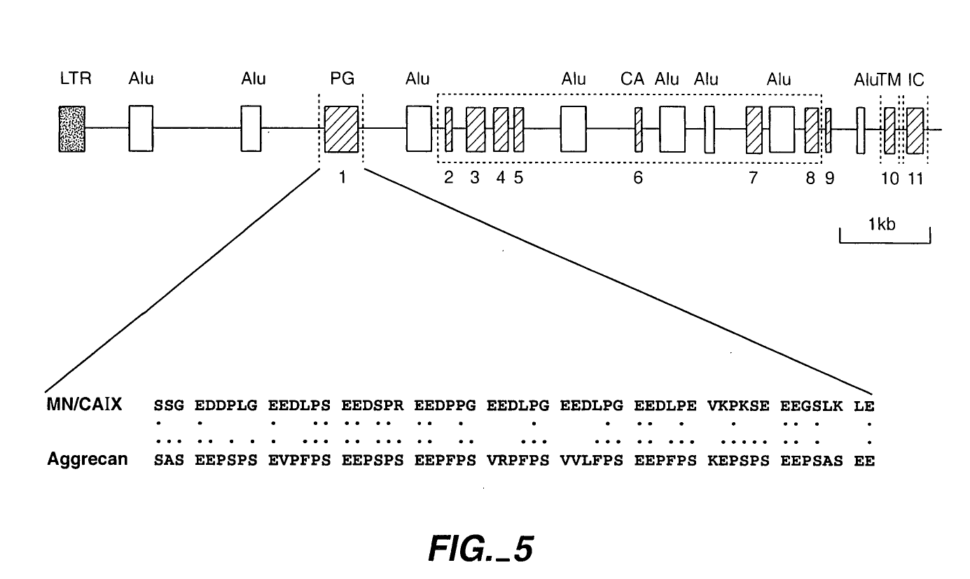

The complete sequence of the overlapping clones contains 10,898 bp (SEQ ID NO: 5). FIG. 5 depicts the organization of the human MN gene, showing the location of all 11 exons as well as the 2 upstream and 6 intronic Alu repeat elements. All the exons are small, ranging from 27 to 191 bp, with the exception of the first exon which is 445 bp. The intron sizes range from 89 to 1400 bp. The CA domain is encoded by exons 2-8, while the exons 1, 10 and 11 correspond respectively to the proteoglycan-like domain, the transmembrane anchor and cytoplasmic tail of the MN/CA IX protein. Table 1 below lists the splice donor and acceptor sequences that conform to consensus splice sequences including the AG-GT motif [Mount, Nucleic Acids Res. 10: 459-472 (1982)].

TABLE 1
Exon-Intron Structure of the Human MN Gene

| | | Genomic | SEQ | 5′splice | SEQ |
|---|---|---|---|---|---|
| Exon | Size | Position** | ID NO | donor | ID NO |
| 1 | 445 | *3507-3951 | 23 | AGAAG gtaagt | 62 |
| 2 | 30 | 5126-5155 | 24 | TGGAG gtgaga | 63 |
| 3 | 171 | 5349-5519 | 25 | CAGTC gtgagg | 64 |
| 4 | 143 | 5651-5793 | 26 | CCGAG gtgagc | 65 |
| 5 | 93 | 5883-5975 | 27 | TGGAG gtacca | 66 |
| 6 | 67 | 7376-7442 | 28 | GGAAG gtcagt | 67 |
| 7 | 158 | 8777-8934 | 29 | AGCAG gtgggc | 68 |
| 8 | 145 | 9447-9591 | 30 | GCCAG gtacag | 69 |
| 9 | 27 | 9706-9732 | 31 | TGCTG gtgagt | 70 |
| 10 | 82 | 10350-10431 | 32 | CACAG gtatta | 71 |
| 11 | 191 | 10562-10752 | 33 | ATAAT end | |
| | | Genomic | SEQ | 3′splice | SEQ |
| Intron | Size | Position** | ID NO | acceptor | ID NO |
| 1 | 1174 | 3952-5125 | 34 | atacag GGGAT | 72 |
| 2 | 193 | 5156-5348 | 35 | ccccag GCGAC | 73 |
| 3 | 131 | 5520-5650 | 36 | acgcag TGCAA | 74 |
| 4 | 89 | 5794-5882 | 37 | tttcag ATCCA | 75 |
| 5 | 1400 | 5976-7375 | 38 | ccccag GAGGG | 76 |
| 6 | 1334 | 7443-8776 | 39 | tcacag GCTCA | 77 |
| 7 | 512 | 8935-9446 | 40 | ccctag CTCCA | 78 |
| 8 | 114 | 9592-9705 | 41 | ctccag TCCAG | 79 |
| 9 | 617 | 9733-10349 | 42 | tcgcag GTGACA | 80 |
| 10 | 130 | 10432-10561 | 43 | acacag AAGGG | 81 |

**positions are related to nt numbering in whole genomic sequence including the 5′ flanking region [FIG. 2A-F]
*number corresponds to transcription initiation site determined below by RNase protection assay

Mapping of MN Gene Transcription Initiation and Termination Sites

Zavada et al., WO 95/34650 describes the process of mapping the MN gene transcription initiation and termination sites. A RNase protection assay was used for fine mapping of the 5′ end of the MN gene. The probe was a uniformly labeled 470 nucleotide copy RNA (nt −205 to +265) [SEQ ID NO: 50], which was hybridized to total RNA from MN-expressing HeLa and CGL3 cells and analyzed on a sequencing gel. That analysis has shown that the MN gene transcription initiates at multiple sites, the 5′ end of the longest MN transcript being 30 nt longer than that previously characterized by RACE.

Characterization of the 5′ Flanking Region

The Bd3 genomic clone isolated from human fetal brain cosmid library was found to cover a region of 3.5 kb upstream from the transcription start site of the MN gene. It contains no significant coding region. Two Alu repeats are situated at positions −2587 to −2296 [SEQ ID NO: 51] and −1138 to −877 [SEQ ID NO: 52] (with respect to the transcription start determined by RNP).

Nucleotide sequence analysis of the DNA 5′ to the transcription start (from nt −507) revealed no recognizable TATA box within the expected distance from the beginning of the first exon. However, the presence of potential binding sites for transcription factors suggests that this region might contain a promoter for the MN gene. There are several consensus sequences for transcription factors AP1 and AP2 as well as for other regulatory elements, including a p53 binding site [Locker and Buzard, J., DNA Sequencinq and Mapping, 1: 3-11 (1990); Imagawa et al. Cell, 51: 251-260 (1987); El Deiry et al., Nat. Genet., 1: 44-49 (1992)]. Although the putative promoter region contains 59.3% C+G, it does not have additional attributes of CpG-rich islands that are typical for TATA-less promoters of housekeeping genes [Bird, Nature, 321: 209-213 (1986)]. Another class of genes lacking TATA box utilizes the initiator (Inr) element as a promoter. Many of these genes are not constitutively active, but they are rather regulated during differentiation or development. The Inr has a consensus sequence of PyPyPyCAPyPyPyPyPy [SEQ ID NO: 20] and encompasses the transcription start site [Smale and Baltimore, Cell, 57: 103-113 (1989)]. There are two such consensus sequences in the MN putative promoter; however, they do not overlap the transcription start (FIG. 6).

An interesting region was found in the middle of the MN gene. The region is about 1.4 kb in length [nt 4,600-6,000 of the genomic sequence; SEQ ID NO: 44] and spans from the 3′ part of the 1st intron to the end of the 5th exon. The region has the character of a typical CpG-rich island, with 62.8% C+G content and 82 CpG: 131 GpC dinucleotides. Moreover, there are multiple putative binding sites for transcription factors AP2 and Sp1 [Locker and Buzard, supra; Briggs et al., Science, 234: 47-52 (1986)] concentrated in the center of this area. Particularly the 3rd intron of 131 bp in length contains three Sp1 and three AP2 consensus sequences. That data indicates the possible involvement of that region in the regulation of MN gene expression. However, functionality of that region, as well as other regulatory elements found in the proposed 5′ MN promoter, remains to be determined.

MN Promoter

Study of the MN promoter has shown that it is TATA-less and contains regulatory sequences for AP-1, AP-2, as well as two p53 binding sites. The sequence of the 5′ end of the 3.5 kb flanking region upstream of the MN gene has shown extensive homology to LTR of HERV-K endogenous retroviruses. Basal transcription activity of the promoter is very weak as proven by analyses using CAT and neo reporter genes. However, expression of the reporter genes is severalfold increased when driven from the 3.5 kb flanking region, indicating involvement of putative enhancers.

Functional characterization of the 3.5 kb MN 5′ upstream region by deletion analysis lead to the identification of the [−173, +31] fragment [SEQ ID NO: 19] (also alternatively, but less preferably, the nearly identical −172, +31 fragment [SEQ ID NO: 84]) as the MN promoter. In vitro DNase I footprinting revealed the presence of five protected regions (PR) within the MN promoter. Detailed deletion analysis of the promoter identified PR 1 and 2 (numbered from the transcription start) as the most critical for transcriptional activity. PR4 [SEQ ID NO: 102] negatively affected transcription as its deletion led to increased promoter activity and was confirmed to function as a promoter-, position- and orientation-independent silencer element. Mutational analysis indicated that the direct repeat AGGGCacAGGGC [SEQ ID NO: 103] is required for efficient repressor binding. Two components of the repressor complex (35 and 42 kDa) were found to be in direct contact with PR4 by UV crosslinking. Increased cell density, known to induce MN expression, did not affect levels of PR4 binding in HeLa cells. Significantly reduced repressor level seems to be responsible for MN up-regulation in the case of tumorigenic CGL3 as compared to non-tumorigenic CGL1 HeLa x normal fibroblast hybrid cells.

### Utility of MN Promoter and MN Promoter Fragments as Tumor-Specific Promoters for Gene Therapy

Being investigated is whether the MN gene promoter and MN/CA9 promoter fragments can be used as tumor-specific promoters to drive the expression of a suicide gene [for example, thymidine kinase (tk) of HSV)] and mediate the direct and bystander killing of tumor cells. HSVtk gene transferred to tumor cells converts nucleoside analogue ganciclovir (GCV) to toxic triphosphates and mediates the death of transduced and also neighboring tumor cells. The control of HSVtk by the MN gene promoter or a MN/CA9 promoter fragment would allow its expression only in tumor cells, which are permissive for the biosynthesis of MN protein, and selectively kill such tumor cells, but not normal cells in which MN expression is repressed.

A plasmid construct in which HSVtk was cloned downstream of the MN promoter region Bd3, containing both proximal and distant regulatory elements of MN, was prepared. That plasmid pMN-HSVtk was transfected to Rat2TK− cells and C33 human cervical carcinoma cells using calcium phosphate precipitation and lipofection, respectively. Transfectants were tested for expression of HSVtk and GVC sensitivity. Analysis of the transfectants has shown the remarkable cytotoxic in vitro effect of GVC even in low concentrations (up to 95% of cells killed).

Polyclonal rabbit antiserum against HSVtk, using fusion protein with GST in pGEX-3X, has been prepared to immunodetect HSVtk synthesized in transfected cells. This model system is being studied to estimate the bystander effect, the inhibition of cloning efficiency and invasiveness of transduced and GVC-treated cells to collagen matrices. A recombinant retroviral vector with the MN promoter-driven HSVtk is to be prepared to test its in vivo efficacy using an animal model (e.g., SCID-mouse).

MN Promoter Analysis

Since the MN promoter is weak, a classical approach to study it would be limited due to the relatively low efficiency of transient transfections (up to 10%). Therefore, stable clonal cell lines expressing constructs containing the MN promoter fused to the CAT gene were prepared. In such clonal lines, 100% of the cells express the CAT gene driven from the MN promoter, and thus, the activity of the promoter is detectable easier than in transient experiments. Also, the promoter activity can be analysed repeatedly in the same cells under different conditions or treated by different factors and drugs. This approach allows for the study of the mechanisms underlying MN regulation at the level of transcription initiation.

Several types of transfections were performed with promoter constructs linked to a reporter CAT gene (calcium precipitation, DEAE dextran combined with DMSO shock and/or chloroquine, as well as electroporation), using different methods of CAT activity assay (scintillation method, thin layer chromatography) and several recipient cell lines differing in the level of MN expression and in transfection efficiency (HeLa, SiHa, CGL3, KATO III, Rat2TK⁻ and C33 cells). Activity of the MN promoter was detected preferably by the electroporation of CGL3 cells and thin layer chromatography. Further preferably, C33 cells cotransfected with MN promoter-CAT constructs and pSV2neo were used.

1. To detect basal activity of the MN promoter and to estimate the position of the core promoter, expression of the CAT gene from constructs pMN1 to pMN7 after transfection to CGL3 cells was analyzed. Plasmids with progressive 5′ deletions were transfected into CGL3 cells and activity was analyzed by CAT assay. [8 μg of DNA was used for transfection in all cases except pBLV-LTR (2 μg).]

Only very weak CAT activity was detected in cells transfected by pMN1 and pMN2 (containing respectively 933 bp and 600 bp of the promoter sequence). A little higher activity was exhibited with the constructs pMN3, pMN4 and pMN6 (containing respectively 446 bp, 243 bp and 58 bp of the promoter). A slight peak of activity was obtained with pMN5 (starting at position −172 with respect to the transcription start.) Thus, the function of the MN core promoter can be assigned to a region of approximately 500 bp immediately upstream from the MN transcription initiation site.

Interestingly, the activity of the large Bd3 region (covering 3.5 kbp upstream of the transcription start) was severalfold higher than the activity of the core promoter. However, its level was still much lower than that exhibited by a positive control, i.e., BLV-LTR transactivated by Tax, and even lower than the activity of BLV-LTR without transactivation. That the activity of Bd3 was elevated in comparison to the core promoter suggests the presence of some regulatory elements. Such elements are most probably situated in the sequence between pMN1 and Bd3 (i.e. from −1 kbp to −3.5 kbp) [SEQ ID NO: 53]. The cloning and transfection of several deletion versions of Bd3 covering the indicated region can be used to determine the location of the putative regulatory elements.

Similar results were obtained from transfecting KATO III cells with Bd3 and pMN4. The transfected cells expressed a lower level of MN than the CGL3 cells. Accordingly, the activity of the MN promoter was found to be lower than in CGL3 cells.

2. In a parallel approach to study the MN promoter, an analysis based on G418 selection of cells transfected by plasmids containing the promoter of interest cloned upstream from the neo gene was made. This approach is suitable to study weak promoters, since its sensitivity is much higher than that of a standard CAT assay. The principle underlying the method is as follows: an active promoter drives expression of the neo gene which protects transfected cells from the toxic effect of G418, whereas an inactive promoter results in no neo product being made and the cells transfected thereby die upon the action of G418. Therefore, the activity of the promoter can be estimated according to the number of cell colonies obtained after two weeks of selection with G418. Three constructs were used in the initial experiments—pMN1neo, pMN4neo and pMN7neo. As pMN7neo contains only 30 bp upstream of the transcription start site, it was considered a negative control. As a positive control, pSV2neo with a promoter derived from SV40 was used. Rat2TK⁻ cells were chosen as the recipient cells, since they are transfectable with high efficiency by the calcium precipitation method.

After transfection, the cells were subjected to two weeks of selection. Then the medium was removed, the cells were rinsed with PBS, and the colonies were rendered visible by staining with methylene blue. The results obtained from three independent experiments corroborated the data from the CAT assays. The promoter construct pMN4neo exhibited higher transcriptional activity than pMN1 neo. However, the difference between the positive control and pMN4neo was not so striking as in the CAT assay. That may have been due to both lower promoter activity of pSV2neo compared to Tax-transactivated pBLV-LTR and to different conditions for cell growth after transfection. From that point of view, stable transfection is probably more advantageous for MN expression, since the cells grow in colonies with close cell to cell contact, and the experiment lasts much longer, providing a better opportunity to detect promoter activity.

3. Stable transfectants expressing MN promoter-CAT chimeric genes were prepared by the cotransfection of relevant plasmids with pSV2neo. As recipient cells, HeLa cells were used first. However, no clones expressing the promoter-CAT constructs were obtained. That negative result was probably caused by homologic recombination of the transfected genomic region of MN (e.g. the promoter) with the corresponding endogenous sequence. On the basis of that experience, C33 cells derived from a HPV-negative cervical carcinoma were used. C33 cells do not express MN, since during the process of tumorigenesis, they lost genetic material including chromosomal region 9p which contains the MN gene. In these experiments, the absence of the MN gene may represent an advantage as the possibility of homologic recombinations is avoided.

C33 Cells Transfected with MN Promoter-CAT Constructs

C33 cells expressing the CAT gene under MN promoter regions Bd3 (−3500/+31) [SEQ ID NO: 83] and MN5 (−172/+31) [SEQ ID NO: 84] were used for initial experiments to analyze the influence of cell density on the transcriptional activity of the MN promoter. The results indicated that signals generated after cells come into close contact activate transcription of the CAT protein from the MN promoter in proportion to the density of the cell culture. Interestingly, the data indicated that the MN protein is not required for this phase of signal transduction, since the influence of density is clearly demonstrated in MN-negative C33 cells. Rather, it appears that MN protein acts as an effector molecule produced in dense cells in order to perform a certain biological function (i.e., to perturb contact inhibition). Also interestingly, the MN promoter activity is detectable even in very sparse cell cultures suggesting that MN is expressed at a very low level also is sparse subconfluent culture.

Deletion Variants. Deletion variants of the Bd3-CAT promoter construct were then prepared. The constructs were cotransfected with pSV2neo into C33 cervical cells. After selection with G418, the whole population of stably transfected cells were subjected to CAT ELISA analysis. Expression of the deletion constructs resulted in the synthesis of similar levels of CAT protein to that obtained with the Bd3-CAT construct. On the basis of that preliminary data, the inventors proposed that sequences stimulating transcription of MN are located between −3506 and −3375 bp [SEQ ID NO: 85] upstream from the transcription start. That is the sequence exhibiting homology to HERV-K LTR.

However, transient transfection studies in CGL3 cells repeatedly revealed that the LTR region is not required for the enhancement of basal MN promoter activity. Further, results obtained in CGL3 cells indicate that the activating element is localized in the region from −933 to −2179 [SEQ ID NO: 97] with respect to transcription initiation site (the position of the region having been deduced from overlapping sequences in the Bd3 deletion mutants).

Interaction of Nuclear Proteins with MN Promoter Sequences

In order to identify transcription factors binding to the MN promoter and potentially regulating its activity, a series of analyses using an electrophoretic mobility shift assay (EMSA) and DNase I footprinting analysis (FTP) were performed.

EMSA

In the EMSA, purified promoter fragments MN4 (−243/+31) [SEQ ID NO: 86], MN5 (−172/+31) [SEQ ID NO: 84], MN6 (−58/+31) [SEQ ID NO: 87] and MN7 (−30/+30) [SEQ ID NO: 88], labeled at the 3′ ends by Klenow enzyme, were allowed to interact with proteins in nuclear extracts prepared from CGL1 and CGL3 cells. [40 μg of nuclear proteins were incubated with 30,000 cpm end-labeled DNA fragments in the presence of 2 μg poly(dIdC).] DNA-protein complexes were analysed by PAGE (native 6%), where the complexes created extra bands that migrated more slowly than the free DNA fragments, due to the shift in mobility which is dependent on the moiety of bound protein.

The EMSA of the MN4 and MN5 promoter fragments revealed several DNA-protein complexes; however, the binding patterns obtained respectively with CGL1 and CGL3 nuclear extracts were not identical. There is a single CGL-1 specific complex.

The EMSA of the MN6 promoter fragment resulted in the formation of three identical complexes with both CGL1 and CGL3 nuclear extracts, whereas the MN7 promoter fragment did not bind any nuclear proteins.

The EMSA results indicated that the CGL1 nuclear extract contains a specific factor, which could participate in the negative regulation of MN expression in CGL1 cells. Since the specific DNA-protein complex is formed with MN4 (−243/+31) [SEQ. ID NO.: 86] and MN5 (−172/+31) [SEQ. ID NO.: 84] promoter fragments, but not with MN6 (−58/+31) [SEQ ID NO: 87], it appears that the binding site of the protein component of that specific complex is located between −173 and −58 bp [SEQ. ID NO.: 89] with respect to transcription initiation.

The next step was a series of EMSA analyses using double stranded (ds) oligonucleotides designed according to the protected regions in FTP analysis. A ds oligonucleotide derived from the protected region PR2 [covering the sequence from −72 to −56 bp (SEQ ID NO: 98)] of the MN promoter provided confirmation of the binding of the AP-1 transcription factor in competitive EMSA using commercial ds olignucleotides representing the binding site for AP-1.

EMSA of ds oligonucleotides derived from the protected regions of PR1 [−46 to −24 bp (SEQ ID NO: 99)], PR2 [−72 to −56 bp (SEQ ID NO: 98)], PR3 [−102 to −85 (SEQ ID NO: 100)] and PR5 [−163 to −144 (SEQ ID NO: 101)] did not reveal any differences in the binding pattern of nuclear proteins extracted from CGL1 and CGL3 cells, indicating that those regions do not bind crucial transcription factors which control activation of the MN gene in CGL3, or its negative regulation in CGL1. However, EMSA of ds oligonucleotides from the protected region PR4 [−133 to −108; SEQ ID NO: 102] repeatedly showed remarkable quantitative differences between binding of CGL1 and CGL3 nuclear proteins. CGL1 nuclear proteins formed a substantially higher amount of DNA-protein complexes, indicating that the PR4 region contains a binding site for specific transcription factor(s) that may represent a negative regulator of MN gene transcription in CGL1 cells. That fact is in accord with the previous EMSA data which showed CGL-1 specific DNA-protein complex with the promoter fragments pMN4 (−243/+31; SEQ ID NO: 86) and pMN5 (−172/+31; SEQ ID NO: 84), but not with pMN6 (−58/+31; SEQ ID NO: 87).

To identify the protein involved or the formation of a specific complex with the MN promoter in the PR4 region, relevant ds oligonucleotides covalently bound to magnetic beads will be used to purify the corresponding transcription factor. Alternatively the ONE Hybrid System® [Clontech (Palo Alto, Calif. (USA)] will be used to search for and clone transcription factors involved in regulation of the analysed promoter region. A cDNA library from HeLa cells will be used for that investigation.

FTP

To determine the precise location of cis regulatory elements that participate in the transcriptional regulation of the MN gene, FTP was used. Proteins in nuclear extracts prepared respectively from CGL1 and CGL3 cells were allowed to interact with a purified ds DNA fragment of the MN promoter (MN4, −243/+31) [SEQ ID NO: 86] which was labeled at the 5′ end of one strand. [MN4 fragments were labeled either at Xho1 site (−243/+31*) or at Xba1 site (*−243/+31).] The DNA-protein complex was then subjected to DNase I attack, which causes the DNA chain to break at certain bases if they are not in contact with proteins. [A control used BSA instead of DNase.] Examination of the band pattern of the denatured DNA after gel electrophoresis [8% denaturing gel] indicates which of the bases on the labeled strand were protected by protein.

FTP analysis of the MN4 promoter fragment revealed 5 regions (I-V) protected at both the coding and noncoding strand, as well as two regions (VI and VII) protected at the coding strand but not at the noncoding strand. FIG. 6 indicates the general regions on the MN promoter that were protected.

The sequences of the identified protected regions (PR) were subjected to computer analysis using the SIGNALSCAN program to see if they corresponded to known consensus sequences for transcription factors. The data obtained by that computer analyses are as follows:
PR I—coding strand—AP-2, p53, GAL4 noncoding strand—JCV-repeated
PR II—coding strand—AP-1, CGN4 noncoding strand—TCF-1, dFRA, CGN4
PR III—coding strand—no known consensus sequence, only partial overlap of AP1 noncoding strand—2 TCF-1 sites
PR IV—coding strand—TCF-1, ADR-1 noncoding strand—CTCF, LF-A1, LBP-1
PR V—coding strand—no known consensus motif noncoding strand—JCV repeated
PR VI—coding strand—no known consensus motif noncoding strand—T antigen of SV 40, GAL4
PR VII—coding strand—NF-uE4, U2snRNA.2 noncoding strand—AP-2, IgHC.12, MyoD.

In contrast to EMSA, the FTP analysis did not find any differences between CGL1 and CGL3 nuclear extracts. However, the presence of specific DNA-protein interactions detected in the CGL1 nuclear extracts by EMSA could have resulted from the binding of additional protein to form DNA protein-protein complex. If that specific protein did not contact the DNA sequence directly, its presence would not be detectable by FTP.

### Sequence Similarities

Computer analysis of the MN cDNA sequence was carried out using DNASIS and PROSIS (Pharmacia Software packages). GenBank, EMBL, Protein Identification Resource and SWISS-PROT databases were searched for all possible sequence similarities. In addition, a search for proteins sharing sequence similarities with MN was performed in the MIPS databank with the FastA program [Pearson and Lipman, PNAS (USA), 85: 2444 (1988)].

The proteoglycan-like domain [aa 53-111 (SEQ ID NO: 45)], which is between the signal peptide and the CA domain, shows significant homology (38% identity and 44% positivity) with a keratan sulphate attachment domain of a human large aggregating proteoglycan aggrecan [Doege et al., J. Biol. Chem., 266: 894-902 (1991)].

The CA domain [aa 135-391 (SEQ ID NO: 46)] is spread over 265 aa and shows 38.9% amino acid identity with the human CA VI isoenzyme [Aldred et al., Biochemistry, 30: 569-575 (1991)]. The homology between MN/CA IX and other isoenzymes is as follows: 35.2% with CA II in a 261 aa overlap [Montgomery et al., Nucl. Acids. Res., 15: 4687 (1987)], 31.8% with CA I in a 261 aa overlap [Barlow et al., Nucl. Acids Res., 15: 2386 (1987)], 31.6% with CA IV in a 266 aa overlap [Okuyama et al., PNAS (USA) 89: 1315-1319 (1992)], and 30.5% with CA III in a 259 aa overlap (Lloyd et al., Genes. Dev., 1: 594-602 (1987)].

In addition to the CA domain, MN/CA IX has acquired both N-terminal and C-terminal extensions that are unrelated to the other CA isoenzymes. The amino acid sequence of the C-terminal part, consisting of the transmembrane anchor and the intracytoplasmic tail, shows no significant homology to any known protein sequence.

The MN gene was clearly found to be a novel sequence derived from the human genome. The overall sequence homology between the cDNA MN sequence and cDNA sequences encoding different CA isoenzymes is in a homology range of 48-50% which is considered by ones in the art to be low. Therefore, the MN cDNA sequence is not closely related to any CA cDNA sequences.

Only very closely related nt sequences having a homology of at least 80-90% would hybridize to each other under stringent conditions. A sequence comparison of the MN cDNA sequence shown in FIG. 1 and a corresponding cDNA of the human carbonic anhydrase II (CA II) showed that there are no stretches of identity between the two sequences that would be long enough to allow for a segment of the CA II cDNA sequence having 25 or more nucleotides to hybridize under stringent hybridization conditions to the MN cDNA or vice versa.

A search for nt sequences related to MN gene in the EMBL Data Library did not reveal any specific homology except for 6 complete and 2 partial Alu-type repeats with homology to Alu sequences ranging from 69.8% to 91% [Jurka and Milosavljevic, J. Mol. Evol. 32: 105-121 (1991)]. Also a 222 bp sequence proximal to the 5′ end of the genomic region is shown to be closely homologous to a region of the HERV-K LTR.

In general, nucleotide sequences that are not in the Alu or LTR-like regions, of preferably 25 bases or more, or still more preferably of 50 bases or more, can be routinely tested and screened and found to hybridize under stringent conditions to only MN nucleotide sequences. Further, not all homologies within the Alu-like MN genomic sequences are so close to Alu repeats as to give a hybridization signal under stringent hybridization conditions. The percent of homology between MN Alu-like regions and a standard Alu-J sequence are indicated as follows:

| Region of Homology within | | |
|---|---|---|
| MN Genomic Sequence | SEQ. | |
| [SEQ ID NO: 5; | ID. | |
| FIG. 2A-F] | NOS. | |
| | | % Homology to |
| | | Entire Alu-J |
| | | Sequence |
| 921-1212 | 54 | 89.1% |
| 2370-2631 | 55 | 78.6% |
| 4587-4880 | 56 | 90.1% |
| 6463-6738 | 57 | 85.4% |
| 7651-7939 | 58 | 91.0% |
| 9020-9317 | 59 | 69.8% |
| | | % Homology to |
| | | One Half of |
| | | Alu-J Sequence |
| 8301-8405 | 60 | 88.8% |
| 10040-10122 | 61 | 73.2%. |

### MN Proteins and/or Polypeptides

The phrase “MN proteins and/or polypeptides” (MN proteins/polypeptides) is herein defined to mean proteins and/or polypeptides encoded by an MN gene or fragments thereof. An exemplary and preferred MN protein according to this invention has the deduced amino acid sequence shown in FIG. 1. Preferred MN proteins/polypeptides are those proteins and/or polypeptides that have substantial homology with the MN protein shown in FIG. 1. For example, such substantially homologous MN proteins/polypeptides are those that are reactive with the MN-specific antibodies of this invention, preferably the Mabs M75, MN12, MN9 and MN7 or their equivalents.

A “polypeptide” or “peptide” is a chain of amino acids covalently bound by peptide linkages and is herein considered to be composed of 50 or less amino acids. A “protein” is herein defined to be a polypeptide composed of more than 50 amino acids. The term polypeptide encompasses the terms peptide and oligopeptide.

MN proteins exhibit several interesting features: cell membrane localization, cell density dependent expression in HeLa cells, correlation with the tumorigenic phenotype of HeLa x fibroblast somatic cell hybrids, and expression in several human carcinomas among other tissues. MN protein can be found directly in tumor tissue sections but not in general in counterpart normal tissues (exceptions noted infra as in normal gastric mucosa and gallbladder tissues). MN is also expressed sometimes in morphologically normal appearing areas of tissue specimens exhibiting dysplasia and/or malignancy. Taken together, these features suggest a possible involvement of MN in the regulation of cell proliferation, differentiation and/or transformation.

It can be appreciated that a protein or polypeptide produced by a neoplastic cell in vivo could be altered in sequence from that produced by a tumor cell in cell culture or by a transformed cell. Thus, MN proteins and/or polypeptides which have varying amino acid sequences including without limitation, amino acid substitutions, extensions, deletions, truncations and combinations thereof, fall within the scope of this invention. It can also be appreciated that a protein extant within body fluids is subject to degradative processes, such as, proteolytic processes; thus, MN proteins that are significantly truncated and MN polypeptides may be found in body fluids, such as, sera. The phrase “MN antigen” is used herein to encompass MN proteins and/or polypeptides.

It will further be appreciated that the amino acid sequence of MN proteins and polypeptides can be modified by genetic techniques. One or more amino acids can be deleted or substituted. Such amino acid changes may not cause any measurable change in the biological activity of the protein or polypeptide and result in proteins or polypeptides which are within the scope of this invention, as well as, MN muteins.

The MN proteins and polypeptides of this invention can be prepared in a variety of ways according to this invention, for example, recombinantly, synthetically or otherwise biologically, that is, by cleaving longer proteins and polypeptides enzymatically and/or chemically. A preferred method to prepare MN proteins is by a recombinant means. Particularly preferred methods of recombinantly producing MN proteins are described below for the GST-MN, MN 20-19, MN-Fc and MN-PA proteins.

Preparation of MN-Specific Antibodies

The term “antibodies” is defined herein to include not only whole antibodies but also biologically active fragments of antibodies, preferably fragments containing the antigen binding regions. Further included in the definition of antibodies are bispecific antibodies that are specific for MN protein and to another tissue-specific antigen.

Zavada et al., WO 93/18152 and WO 95/34650 describe in detail methods to produce MN-specific antibodies, and detail steps of preparing representative MN-specific antibodies as the M75, MN7, MN9, and MN12 monoclonal antibodies. Preferred MN antigen epitopes comprise: aa 62-67 (SEQ ID NO: 10); aa 61-66, aa 79-84, aa 85-90 and aa 91-96 (SEQ ID NO: 91); aa 62-65, aa 80-83, aa 86-89 and aa 92-95 (SEQ ID NO: 92); aa 62-66, aa 80-84, aa 86-90 and aa 92-96 (SEQ ID NO: 93); aa 63-68 (SEQ ID NO: 94); aa 62-68 (SEQ ID NO: 95); aa 82-87 and aa 88-93 (SEQ ID NO: 96); aa 55-60 (SEQ ID NO: 11); aa 127-147 (SEQ ID NO: 12); aa 36-51 (SEQ ID NO: 13); aa 68-91 (SEQ ID NO: 14); aa 279-291 (SEQ ID NO: 15); and aa 435-450 (SEQ ID NO: 16).

Bispecific Antibodies. Bispecific antibodies can be produced by chemically coupling two antibodies of the desired specificity. Bispecific MAbs can preferably be developed by somatic hybridization of 2 hybridomas. Bispecific MAbs for targeting MN protein and another antigen can be produced by fusing a hybridoma that produces MN-specific MAbs with a hybridoma producing MAbs specific to another antigen. For example, a cell (a quadroma), formed by fusion of a hybridoma producing a MN-specific MAb and a hybridoma producing an anti-cytotoxic cell antibody, will produce hybrid antibody having specificity of the parent antibodies. [See. e.g., Immunol. Rev. (1979); Cold Spring Harbor Symposium Quant. Biol., 41: 793 (1977); van Dijk et al., Int. J. Cancer, 43: 344-349 (1989).] Thus, a hybridoma producing a MN-specific MAb can be fused with a hybridoma producing, for example, an anti-T3 antibody to yield a cell line which produces a MN/T3 bispecific antibody which can target cytotoxic T cells to MN-expressing tumor cells.

It may be preferred for therapeutic and/or imaging uses that the antibodies be biologically active antibody fragments, preferably genetically engineered fragments, more preferably genetically engineered fragments from the V_{H} and/or V_{L} regions, and still more preferably comprising the hypervariable regions thereof. However, for some therapeutic uses bispecific antibodies targeting MN protein and cytotoxic cells would be preferred.

Epitopes

The affinity of a MAb to peptides containing an epitope depends on the context, e.g. on whether the peptide is a short sequence (4-6 aa), or whether such a short peptide is flanked by longer aa sequences on one or both sides, or whether in testing for an epitope, the peptides are in solution or immobilized on a surface. Therefore, it would be expected by ones of skill in the art that the representative epitopes described herein for the MN-specific MAbs would vary in the context of the use of those MAbs.

The term “corresponding to an epitope of an MN protein/polypeptide” will be understood to include the practical possibility that, in some instances, amino acid sequence variations of a naturally occurring protein or polypeptide may be antigenic and confer protective immunity against neoplastic disease and/or anti-tumorigenic effects. Possible sequence variations include, without limitation, amino acid substitutions, extensions, deletions, truncations, interpolations and combinations thereof. Such variations fall within the contemplated scope of the invention provided the protein or polypeptide containing them is immunogenic and antibodies elicited by such a polypeptide or protein cross-react with naturally occurring MN proteins and polypeptides to a sufficient extent to provide protective immunity and/or anti-tumorigenic activity when administered as a vaccine.

Epitope for M75 MAb

The M75 epitope is considered to be present in at least two copies within the 6× tandem repeat of 6 amino acids [aa 61-96 (SEQ ID NO: 90)] in the proteglycan domain of the MN protein. Exemplary peptides representing that epitope depending on the context may include the following peptides from that tandem repeat: EEDLPS (SEQ ID NO: 10; aa 62-67); GEEDLP (SEQ ID NO: 91; aa 61-66; aa 79-84; aa 85-90; aa 91-96); EEDL (SEQ ID NO: 92; aa 62-65; aa 80-83; aa 86-89; aa 92-95); EEDLP (SEQ ID NO. 93; aa 62-66; aa 80-84; aa 86-90; aa 92-96); EDLPSE (SEQ ID NO: 94; aa 63-68); EEDLPSE (SEQ ID NO: 95; aa 62-68); and DLPGEE (SEQ ID NO: 96; aa 82-87, aa 88-93).

Other Epitopes

Mab MN9. Monoclonal antibody MN9 (Mab MN9) reacts to the same epitope as Mab M75, as described above. As Mab M75, Mab MN9 recognizes both the GST-MN fusion protein and native MN protein equally well.

Mabs corresponding to Mab MN9 can be prepared reproducibly by screening a series of mabs prepared against an MN protein/polypeptide, such as, the GST-MN fusion protein, against the peptides representing the epitope for Mabs M75 and MN9. Alternatively, the Novatope system [Novagen] or competition with the deposited Mab M75 could be used to select mabs comparable to Mabs M75 and MN9.

Mab MN12. Monoclonal antibody MN12 (Mab MN12) is produced by the mouse lymphocytic hybridoma MN 12.2.2 which was deposited under ATCC HB 11647. Antibodies corresponding to Mab MN12 can also be made, analogously to the method outlined above for Mab MN9, by screening a series of antibodies prepared against an MN protein/polypeptide, against the peptide representing the epitope for Mab MN12. That peptide is aa 55-aa 60 of FIG. 1 [SEQ ID NO: 11]. The Novatope system could also be used to find antibodies specific for said epitope.

Mab MN7. Monoclonal antibody MN7 (Mab MN7) was selected from mabs prepared against nonglycosylated GST-MN as described above. It recognizes the epitope represented by the amino acid sequence from aa 127 to aa 147 [SEQ ID NO: 12] of the FIG. 1 MN protein. Analogously to methods described above for Mabs MN9 and MN12, mabs corresponding to Mab MN7 can be prepared by selecting mabs prepared against an MN protein/polypeptide that are reactive with the peptide having SEQ ID NO: 12, or by the stated alternative means.

### MN-Specific Intrabodies—Targeted Tumor Killing Via Intracellular Expression of MN-Specific Antibodies to Block Transport of MN Protein to Cell Surface

The gene encoding antibodies can be manipulated so that the antigen-binding domain can be expressed intracellularly. Such “intrabodies” that are targeted to the lumen of the endoplasmic reticulum provide a simple and effective mechanism for inhibiting the transport of plasma membrane proteins to the cell surface. [Marasco, W. A., “Review—Intrabodies: turning the humoral immune system outside in or intracellular immunization,” Gene Therapy, 4: 11-15 (1997); Chen et al., “Intracellular antibodies as a new class of therapeutic molecules for gene therapy,” Hum. Gene Ther., 5(5): 595-601 (1994); Mhashilkar et al., EMBO J., 14: 1542-1551 (1995); Mhashilkar et al., J. Virol., 71: 6486-6494 (1997); Marasco (Ed.), Intrabodies: Basic Research and Clinical Gene Therapy Applications, (Springer Life Sciences 1998; ISBN 3-540-64151-3) (summarizes preclinical studies from laboratories worldwide that have used intrabodies); Zanetti and Capra (Eds.), “Intrabodies: From Antibody Genes to Intracellular Communication,” The Antibodies: Volume 4, [Harwood Academic Publishers; ISBN 90-5702-559-0 (Dec. 1997)]; Jones and Marasco, Advanced Drug Delivery Reviews, 31 (1-2): 153-170 (1998); Pumphrey and Marasco, Biodrugs, 9(3): 179-185 (1998); Dachs et al., Oncology Res., 9(6-7); 313-325 (1997); Rondon and Marasco, Ann. Rev. Microbiol., 51: 257-283 (1997)]; Marasco, W. A., Immunotechnology, 1(1): 1-19 (1995); and Richardson and Marasco, Trends in Biotechnology, 13(8): 306-310 (1995).]

MN-specific intrabodies may prevent the maturation and transport of MN protein to the cell surface and thereby prevent the MN protein from functioning in an oncogenic process. Antibodies directed to MN's EC, TM or IC domains may be useful in this regard. MN protein is considered to mediate signal transduction by transferring signals from the EC domain to the IC tail and then by associating with other intracellular proteins within the cell's interior. MN-specific intrabodies could disrupt that association and perturb that MN function.

Inactivating the function of the MN protein could result in reversion of tumor cells to a non-transformed phenotype. [Marasco et al. (1997), supra.] Antisense expression of MN cDNA in cervical carcinoma cells, as demonstrated herein, has shown that loss of MN protein has led to growth suppression of the transfected cells. It is similarly expected that inhibition of MN protein transport to the cell surface would have similar effects. Cloning and intracellular expression of the M75 MAb's variable region is to be studied to confirm that expectation.

Preferably, the intracellularly produced MN-specific antibodies are single-chain antibodies, specifically single-chain variable region fragments or scFv, in which the heavy- and light-chain variable domains are synthesized as a single polypeptide and are separated by a flexible linker peptide, preferably (Gly₄-Ser)₃ [SEQ ID NO: 115].

MN-specific intracellularly produced antibodies can be used therapeutically to treat preneoplastic/neoplastic disease by transfecting preneoplastic/neoplastic cells that are abnormally expressing MN protein with a vector comprising a nucleic acid encoding MN-specific antibody variable region fragments, operatively linked to an expression control sequence. Preferably said expression control sequence would comprise the MN gene promoter.

### Antibody-Mediated Gene Transfer Using MN-Specific Antibodies or Peptides for Targeting MN-Expressing Tumor Cells

An MN-specific antibody or peptide covalently linked to polylysine, a polycation able to compact DNA and neutralize its negative charges, would be expected to deliver efficiently biologically active DNA into an MN-expressing tumor cell. If the packed DNA contains the HSVtk gene under control of the MN promoter, the system would have double specificity for recognition and expression only in MN-expressing tumor cells. The packed DNA could also code for cytokines to induce CTL activity, or for other biologically active molecules. The M75 MAb (or, for example, as a single chain antibody, or as its variable region) is exemplary of such a MN-specific antibody.

### EXAMPLES CONCERNING MN AND HYPOXIA

The following examples are for purposes of illustration only and are not meant to limit the invention in any way.

### Materials and Methods for Examples 1-4

Construction of Reporter Plasmids

To generate plasmids p-506 and p-173, sequences of the MN/CA9 gene between −506 and +43 relative to the transcriptional start site were amplified by PCR from genomic DNA. PCR products were ligated into pGL3-basic, a promoterless and enhancerless luciferase expression vector (Promega). To generate plasmids p-36, MUTI, and MUT2, complementary oligonucleotides with ends corresponding to the 5′ restriction cleavage overhangs of Bg/II and MluI were annealed and ligated into Bg/II/MluI-digested pGL3-basic. Oligonucleotides (sense strand) were: p-36 (forward), 5′-cgcgCTCCCCCACCCAGCTCTCGTTTCCAATGCACGTACAGCCCGTACACACCG-3′; [SEQ ID NO: 112] MUTI (forward), 5′-cgcgCTCCCCCACCCAGCTCTCGTTTCC-MTGCTTTTACAGCCCGTACACACCG-3′; [SEQ ID NO: 113] MUT2 (forward), 5′-cgcgCTCCCCCACCCAGCTCTCGTTTCCAATGCAAGTACAGCCCGTACACACCG-3′ [SEQ ID NO: 114]. Nucleotides introduced for cloning are lowercase; mutations are underlined. All MN/CA9 promoter sequences were confirmed by dideoxy sequence analysis.

Transient Expression Assays

Cells at ˜70% confluence in 60-mm dishes were transfected with 1 μg of a luciferase reporter construct and 0.4 μg of control plasmid, pCMV-βgal (Promega), using FuGENE 6 (Roche Diagnostic) according to the manufacturer's instructions. Cells were then incubated at 20% O₂ for 8 h, followed by 20% or 0.1% O₂ for 16 h.

Luciferase activity was determined in cell lysates using a commercial assay system (Promega) and a TD-20e luminometer (Turner Designs). βgal activity in cell lysates was measured using o-nitrophenyl-β-D-galactopyranoside as substrate in a 0.1 M phosphate buffer (pH 7.0) containing 10 mM KC1, 1 mM MgSO₄, and 30 mM β-mercaptoethanol. To correct for viable transfection efficiencies between experimental conditions, the luciferase: βgal ratio was determined for each sample. For cotransfection assays, cells also received 0.1-1 μg each of pCDNA3/HIF-1α or pCDNA3/HIF-2α containing the entire human HIF-1α or HIF-2α open reading frame, respectively. Transfections were balanced with various amounts of pCDNA3 (Invitrogen) and pCDNA3/HIF-α such that all cells received the same total quantity of DNA.

### Example 1

### Oxygen-Dependent Function of MN/CA9 Promoter

To investigate the unusually tight regulation of MN/CA9 mRNA by hypoxia, the oxygen-dependent function of the MN/CA9 promoter was tested. In the first set of experiments, luciferase reporter genes containing ˜0.5 kb of MN/CA9 5′ flanking sequences (−506 to +43) [SEQ ID NO: 104] and a deletion to nucleotide −173 (−173 to +43) [SEQ ID NO: 111] were tested in transiently transfected HeLa cells. Both constructs showed very low levels of activity in normoxic cells but were induced strongly by hypoxia. By contrast, a similar reporter linked to a minimal SV40 promoter showed no induction by hypoxia.

### Example 2

### Dependence of MN/CA9 Promoter on HIF-1

To test whether these responses were dependent on HIF-1, further transfections were performed using a CHO mutant cell (Ka13) that is functionally defective for the HIF-1α subunit and cannot form the HIF-1 transcriptional complex. [Wood et al., “Selection and analysis of a mutant cell line defective in the hypoxia-inducible factor-1α subunit (HIF-1α),” J. Biol. Chem., 273: 8360-8368 (1998).] In the CHO wild-type parental subline C4.5, the −173 nucleotide promoter [SEQ ID NO: 111] conferred 17-fold transcriptional induction by hypoxia. In contrast, in the HIF-1α-deficient Ka13 subline, this hypoxic induction was absent. Cotransfection of human HIF-1α restored hypoxia-inducible activity to the MN/CA9 promoter in the Ka13 cells and increased normoxic activity in both C4.5 and Ka13. In C4.5 and Ka13 cells at 0.1% O₂, luciferase expression was increased 1.6- and 17-fold, respectively, by cotransfection of human HIF-1α Thus, hypoxia-inducible activity of the MN/CA9 promoter is completely dependent on HIF-1 and strongly influenced by the level of HIF-1α. Activity of the MN/CA9 promoter in Ka13 cells could also be restored by cotransfection of HIF-2α, although normoxic activity was higher and fold induction by hypoxic stimulation was reduced.

### Example 3

### Response of Putative MN/CA9 HRE to Hypoxia

Inspection of the MN/CA9 5′ flanking sequences revealed a consensus HRE beginning 3 bp 5′ to the transriptional start site, oriented on the antisense strand, reading 5′-TACGTGCA-3′ [SEQ ID NO: 105]. To test the importance of this site, a MN/CA9 minimal promoter was constructed containing this sequence (−36 to +14) [SEQ ID NO: 106]. This minimal promoter retained hypoxia-inducible activity in C4.5 cells but had no inducible activity in Ka13 cells. Absolute levels of activity were lower in comparison to the −173 nucleotide promoter [SEQ ID NO: 111] construct, being reduced −8 fold, indicating that although sequences −173 to −36 amplified promoter activity, responsiveness to hypoxia was conveyed by the minimal sequence containing the MN/CA9 HRE.

### Example 4

### Mutational Analysis of MN/CA9 HRE

To confirm the importance of the MN/CA9 HRE, two mutations were made within its core (antisense strand): a 3-bp substitution from CGT→AAA (MUT1), and a single substitution of G→T (MUT2). Both mutations completely ablated hypoxia-inducible activity, although basal activity was preserved or slightly increased for MUT1.

### EXAMPLES CONCERNING MN/CA IX AND RADIOBIOLOGICAL TUMOR HYPOXIA

### Materials and Methods for Examples 5-7

Cell Culture

Early-passage HT 1080 human fibrosarcoma cells and FaDu human pharyngeal carcinoma from the American Type Culture Collection (ATCC, Manassas, Va.) were maintained under standard conditions [Vordermark et al., Int. J. Radiat. Oncol. Biol. Phys. 58: 1242-1250 (2004); Vordermark et al., Cancer Lett. (in press)] as follows: cells were maintained in a Dulbecco's modified Eagle's medium and α-MEM, respectively, supplemented with 10% fetal bovine serum and 100,000 U/L penicillin and 100 mg/L streptomycin (all from Sigma, St Louis, Mo.) with 5% CO₂ in a well-humidified incubator.

For hypoxia experiments, early-passage cells were split and seeded into 80-mm glass petri dishes at 1×10⁶ cells per dish. For experiments analyzing the effect of cell density, 2×10⁵, 1×10⁶ or 5×10⁶ cells were seeded per dish, corresponding to 4,000, 20,000 or 100,000 cells, respectively, per cm². The following day, hypoxia (5%, 1% or 0.1% O₂) was achieved inside a Ruskinn (Cincinnatti, Ohio) Invivo₂ hypoxic workstation before transfer of the cells by calibrating the oxygen probe against air according to the manufacturer's instructions, adjusting the instrument settings to the desired O₂ concentration, 5% CO₂ and 37° C., and subsequent flooding of the chamber with an appropriate gas mixture of pressurized air, N₂ and CO₂ through an automated gas mixing module. Cells were treated under hypoxic conditions for the times indicated. Aerobic conditions were maintained in a well-humidified incubator at 5% CO₂ and 20% O₂. The effect of reoxygenation was analyzed in cells returned to the incubator after 24 h at 0.1% O₂. Cells treated with the chelating agent desferrioxamine (DFO; Sigma) at 100 μM under aerobic conditions for 24 h served as positive controls. Aliquots from one sample of whole-cell lysates or nuclear extracts of HT 1080 cells treated with DFO, for MN/CA IX and HIF-1α experiments, respectively, were stored at −20° C. and run with each Western blot as an additional positive control to allow quantitative comparison between experiments.

In one series of experiments, culture conditions were manipulated to simulate non-hypoxic tumor microenvironment conditions. Medium was exchanged immediately before initiation of hypoxic or control aerobic conditions. To simulate acidosis, pH of DMEM buffered with 20 mM TRIS base, 20 mM MES and 0.52 g/l NaHCO₃ (all from Sigma) was adjusted to 6.7 or, for control conditions, to 7.4. Stability of pH under both aerobic and hypoxic conditions was monitored. Fetal bovine serum (Sigma) was added to the medium at a final concentration of 10% or omitted (0%). Glucose (Sigma) was added at the normal concentration of 5.5 mM or omitted (0 mM). In subsequent experiments, intermediate glucose concentrations were studied.

Western Blot Analysis

Immediately following the respective treatment, cells were placed one ice. For analysis of MN/CA IX protein, whole-cell lysates were prepared in 1% Triton X-100 lysis buffer [Vordermark et al., Neoplasia, 3: 527-534 (2001)]. In a separate series of cell density experiments, nuclear extracts were prepared for HIF-1α analysis as follows: culture dishes were placed on ice, washed once with ice-cold PBS, and lysed in buffer A: 10 mM Tris (pH 7.4), 1 mM EDTA, 0.15 M sodium chloride, 0.5% NP-40 with protease inhibitors 20 μg/mL aprotinin, 5 μg/mL leupeptin, and 1 mM phenylmethane sulfonyl fluoride (PMSF). Lysates were scraped and centigufed for 5 min at 500 g at 4° C. The supernatant was discarded and the pellet suspended in lysis buffer B: 50 mM HEPES (pH 7.9), 0.4 M sodium chloride, 1 mM EDTA, protease inhibitors as above plus 1 mM dithiothreitol. The suspension was centrifuged for 5 min at 20,000 g at 4° C. [Vordermark et al., Int. J. Radiat. Oncol. Biol. Phys. 58: 1242-1250 (2004)]. Protein content of samples was determined using the Bio-Rad DC protein assay (Bio-Rad, Hercules, Calif.). Nuclear extracts (10 μg/lane) were separated by sodium dodecyl sulfate-polyacrylamide gel electrophoresis (SDS-PAGE) (4-12% polyacrylamide gel, Invitrogen, Carlsbad, Calif.) and transferred to a nitrocellulose membrane using a Novex Xcell II tank blotter (both Invitrogen). Membranes were blocked overnight with 4% nonfat dry milk and 0.05% Tween-20 in PBS.

For detection of MN/CA IX protein, membranes were incubated with the mouse monoclonal M75 antibody against MN/CA IX [Pastorekova et al., Virology 187: 620-626 (1992)] at a concentration of 0.75 μg/ml [Kaluz et al., Cancer Res 62: 4469-4477 (2002)] in blocking solution for 2 h at 4° C. and subsequently with the secondary goat anti-mouse IgG (H+L) AP antibody (DAKO, Carpinteria, Calif.) at 1:2000 dilution in blocking solution for 1 h. After treatment with stripping buffer (100 mM β-mercaptoethanol, 2% sodium dodecyl sulphate, 62.5 mM Tris HCl pH 6.7) at 60° C. for 30 min, membranes were reprobed with anti-β-actin mouse monoclonal antibody (Sigma, 1:10,000) and secondary antibody as above as a loading control. For HIF-1α detection, membranes were treated accordingly, with anti-HIF-1α mouse monoclonal antibody (BD Transduction Laboratories, Lexington, Ky.) at 1:500 dilution as primary. Membranes were reprobed as described above, with anti-β-tubulin mouse monoclonal antibody (Sigma, 1:2000 dilution) and secondary antibody as above as a loading control.

Detection was performed with ECL plus Western blotting detection system (Amersham, Piscataway, N.J.), and band density was quantified using Kodak (New Haven, Conn.) 1D software.

MN/CA IX Flow Cytometry, Irradiation and Clonogenic Survival

Following treatment with hypoxic (24 h, 0.1% O₂) or control aerobic conditions, glass dishes were enclosed in custom-made, airtight perspex shells inside the hypoxic workstation and transferred to the linear accelerator. Cells were irradiated with a single dose of 10 Gy (dose rate 2.5 Gy/min, 6 MV photons, room temperature). Aerobic cells were irradiated in similar perspex shells with holes permitting unimpaired air exchange.

Immediately following irradiation, cells were scraped from glass dishes and washed twice in PBS. Cells were counted and mixtures of aerobic and hypoxic cells prepared (0%, 1%, 10%, 50%, 90%, 99% and 100% hypoxic cells). Samples of 2×10⁶ unfixed cells were blocked with PBS containing 5% normal goat serum and 0.1% bovine serum albumin (both Sigma) for 20 min at room temperature. Cells were then incubated with the mouse monoclonal M75 antibody against MN/CA IX (1:100 in above blocking solution), washed twice and incubated with a fluorescein-isothiocyanate-(FITC-) conjugated F(ab′)₂ fragment of goat-anti-mouse antibody (Alexa Fluor 488 from Molecular Probes, Eugene, Oreg.; 1:100 in above blocking solution). Cells were washed twice and resuspended in PBS for analysis.

Flow cytometry was performed on a Becton Dickinson FacsCalibur under standard conditions [Vordermark et al., Int J Radiat Oncol Biol Phys 56: 1184-1193 (2003)]. 10,000 events per sample were acquired. For quantification of the percentage of MN/CA IX-negative and positive populations in mixed samples, regions were defined based on the extension of the populations in unmixed aerobic or hypoxic cell suspensions. Appropriate numbers of cells from above mixed cell suspensions were plated in triplicate into plastic dishes for colony-forming assay. On day 14, cells were stained with crystal violet and colonies consisting of at least 50 cells were counted. Mean (±SEM) plating efficiency was 50.9±3.0% for HT 1080 and 13.6±1.5% for FaDu.

Statistical Analysis

For quantification of MN/CA IX and HIF-1α protein content, the ratio of MN/CA IX/β-actin and HIF-1α/β-tubulin band densities, respectively, of a control sample of DFO-treated HT 1080 cells, aliquots of which were run with each blot, were defined as 100% and used as a reference for quantification. Means±SEM were calculated for each condition. MN/CA IX protein levels were related to a modified oxygen enhancement ratio (OER′) published previously for HT 1080 and FaDu cells [Vordermark et al, Int. J. Radiat. Oncol. Biol. Phys. 58: 1242-1250 (2004)]. This value was calculated from clonogenic survival curves of cells irradiated at different O₂ concentrations, as follows.

For irradiation experiments, the plating efficiency of unirradiated aerobic cells was set to define a surviving fraction of 1 for each treatment day, against which all other conditions were compared. Cell survival data for each condition and cell line were fit to a linear-quadratic equation using Origin version 2.75 (OriginLab, Northampton, Mass.) software. For each condition, D10 (radiation dose producing a surviving fraction of 10%) was calculated. Based on D10 values, a modified oxygen enhancement ratio (OER′) was determined by dividing the D10 for the respective condition by the D10 at 0.1% O₂, which was the most hypoxic condition investigated. This value was denoted OER′, because the oxygen enhancement ratio by definition requires comparision with cell survival under anoxic conditions. [Steel, G. G., Basic Clinical Radiobiology, 2^{nd} ed., London: Arnold; 1997, pp. 158-159].

Differences between specific conditions and correlation between assays were evaluated using Mann-Whitney U test and Spearman-rank test, respectively, using Statistica 6.0 (Statsoft, Tulsa, Okla.) software (p<0.05 considered significant).

### Example 5

MN/CA IX Protein Accumulation under In-Vitro Hypoxia and Stability during Reoxygenation

The time course of MN/CA IX protein levels in both HT 1080 human fibrosarcoma and FaDu human pharyngeal carcinoma cells exposed to in-vitro hypoxia at 5%, 1% or 0.1% O₂, with or without reoxygenation and in comparison with aerobic conditions (negative control) and the hypoxia-mimetic chelating agent desferrioxamine (DFO) is shown in FIG. 10, summarizing Western blot data. In both cell lines, a similar time course was observed with no accumulation of MN/CA IX protein during the first 6 h of hypoxia and increase to maximal levels at 24 h. In HT 1080, MN/CA IX protein was indistinguishable between the O₂ concentrations of 5%, 1% and 0.1%. In FaDu, MN/CA IX was approximately half-maximal under 5% O₂ with comparable maximal values for the 1% and 0.1% conditions. In both cell lines MN/CA IX was stable during 15 min of reoxygenation.

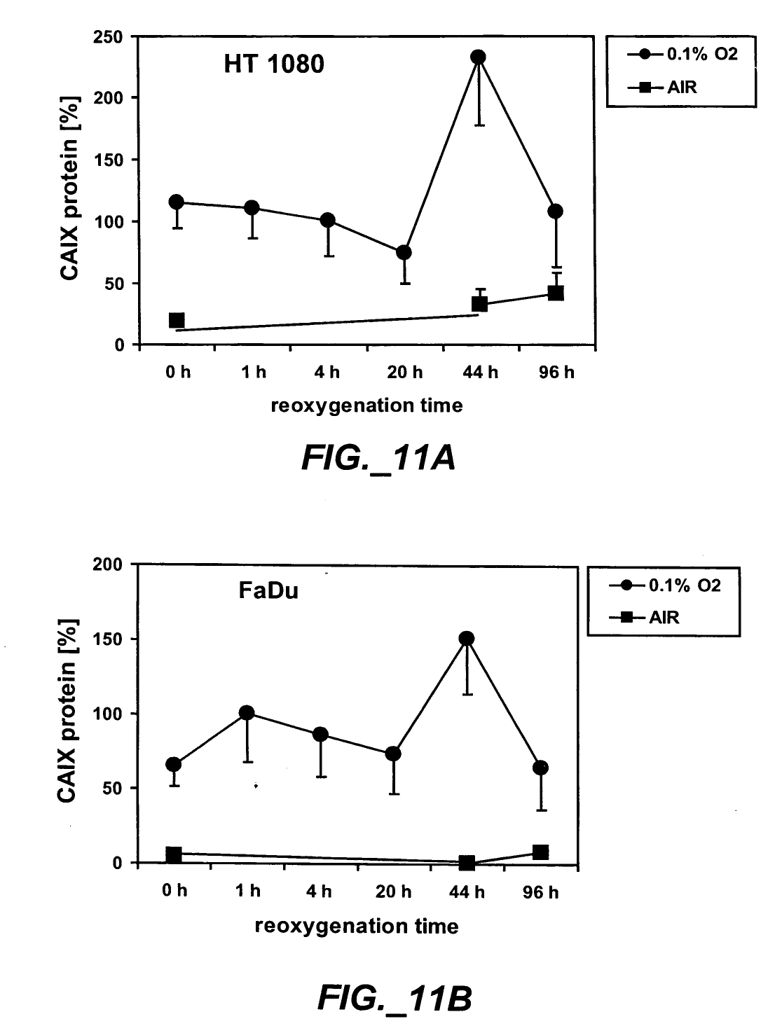

A separate series of experiments showed that MN/CA IX protein was stable during much longer periods of reoxygenation after 24 h at 0.1% O₂, up to 96 h (FIG. 11). In both cell lines, however, long-term incubation under aerobic conditions led to significant MN/CA IX protein levels even in positive control cells never exposed to hypoxia.

To investigate the potential of MN/CA IX to indicate radiobiological tumor hypoxia, the MN/CA IX protein levels after 24 h of hypoxia were compared with the previously published OER′ values, indicators of hypoxic radiation resistance, calculated for both cell lines [Vordermark et al, Int. J. Radiat. Oncol. Biol. Phys., 58: 1242-1250 (2004)]. Whereas in FaDu a reasonable correlation was observed, no correlation was seen in HT 1080 (FIG. 12). This difference is mainly caused by the maximal MN/CA IX protein levels in HT 1080 and half-maximal levels in FaDu at 5% O₂, a condition not associated with significant increase in radiation resistance.

### Example 6

### Effect of Non-Hypoxic Stimuli on MN/CA IX Protein Levels

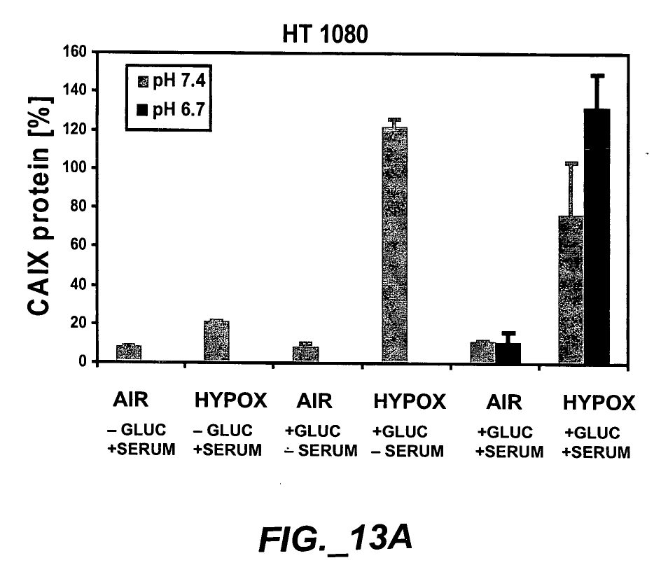

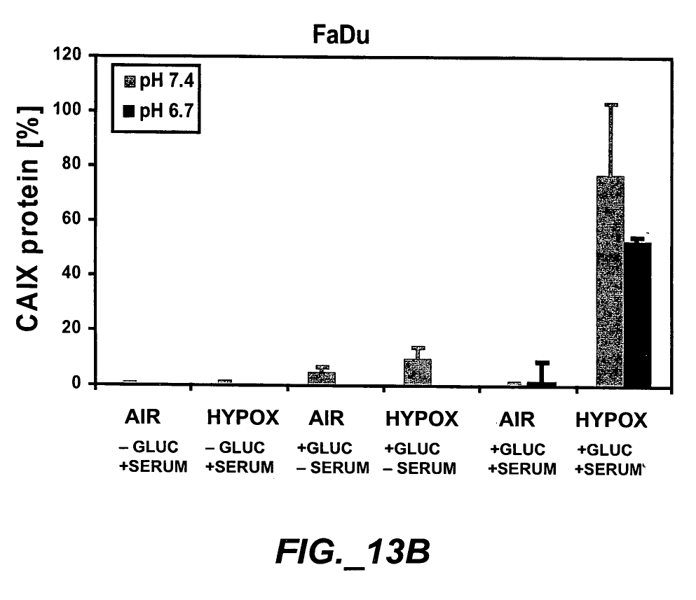

Aerobic exposure of HT 1080 or FaDu cells to glucose-deprived, serum-deprived or acid (pH 6.7) conditions alone had no detectable effect on MN/CA IX protein levels (FIG. 13A-B). Response to severe hypoxia (24 h, 0.1% O₂), however, was strongly modified. In particular, in HT 1080 cells a lack of glucose, but not a lack of serum, abolished the MN/CA IX protein response almost completely (FIG. 13A). In FaDu, both serum and glucose availability were essential for any significant MN/CA9 response to hypoxia (FIG. 13B).

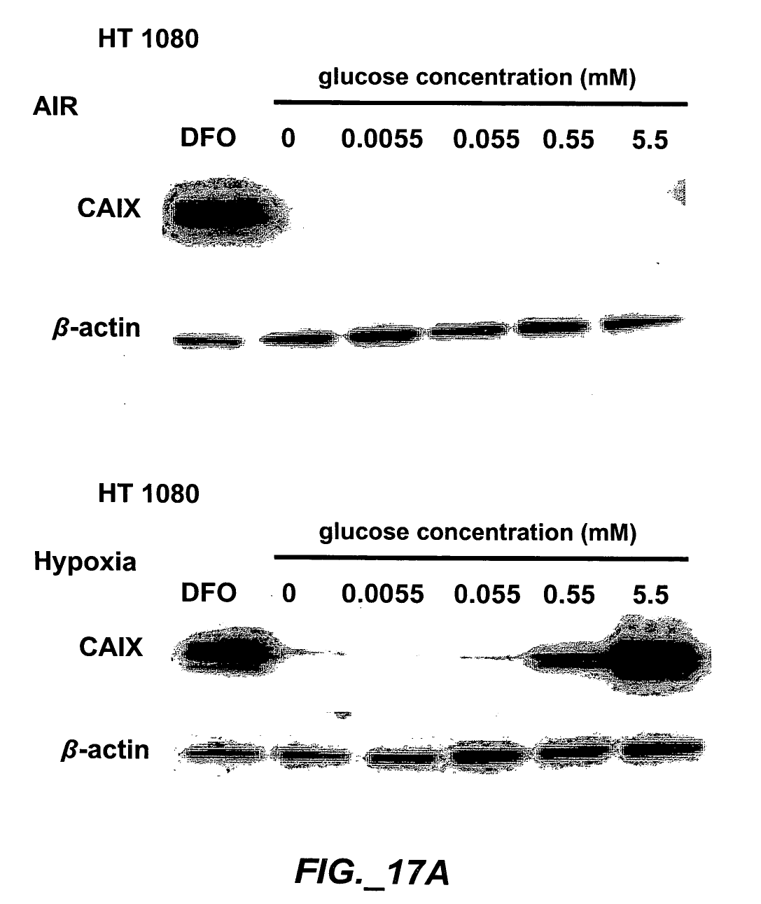

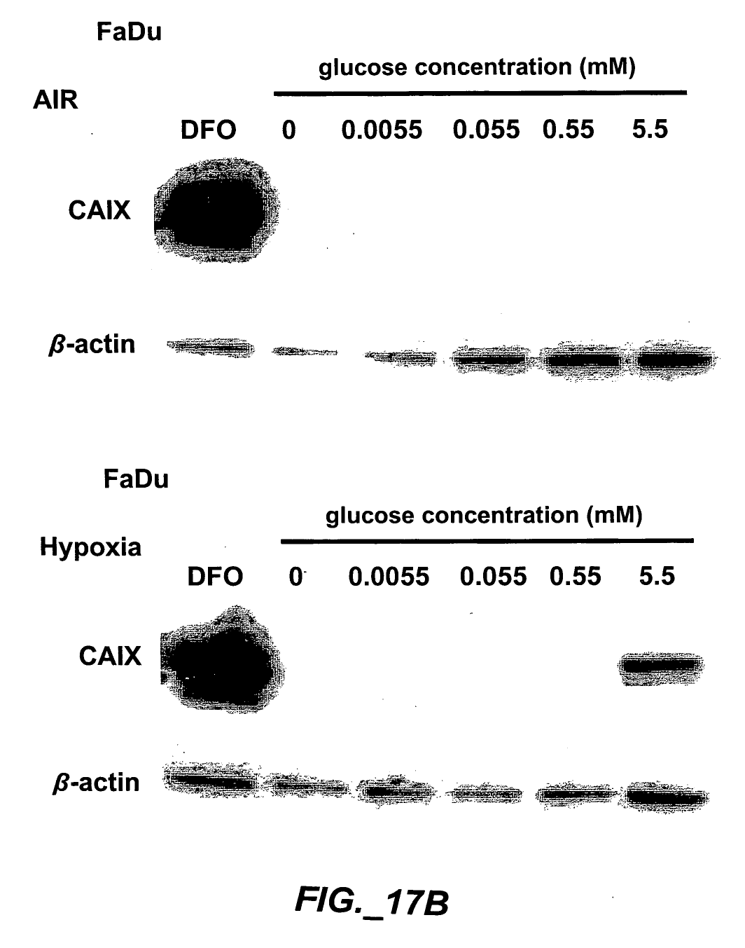

Investigation of different glucose concentrations showed that reduction from the standard concentration of 5.5 mM to 0.55 mM strongly reduced MN/CA IX protein levels under hypoxia in HT 1080 and eliminated the hypoxic response in FaDu cells (FIG. 17A-B).

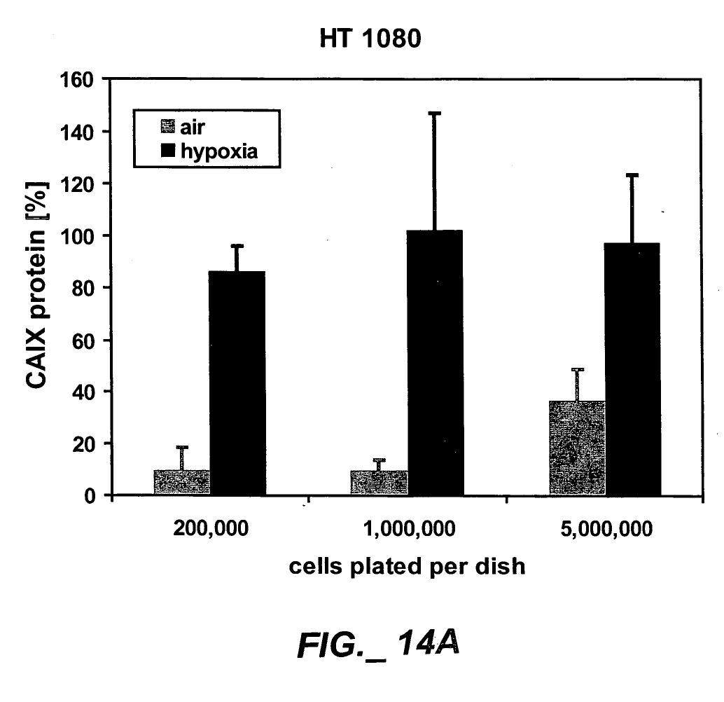

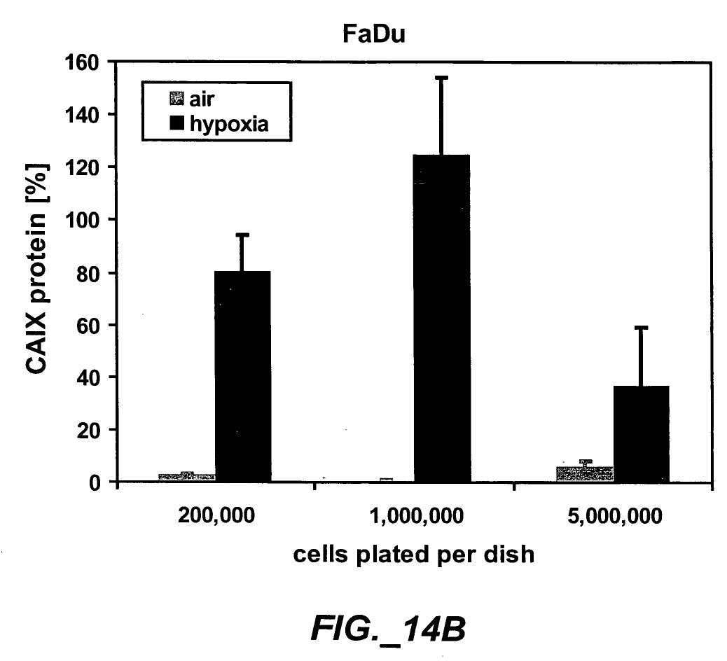

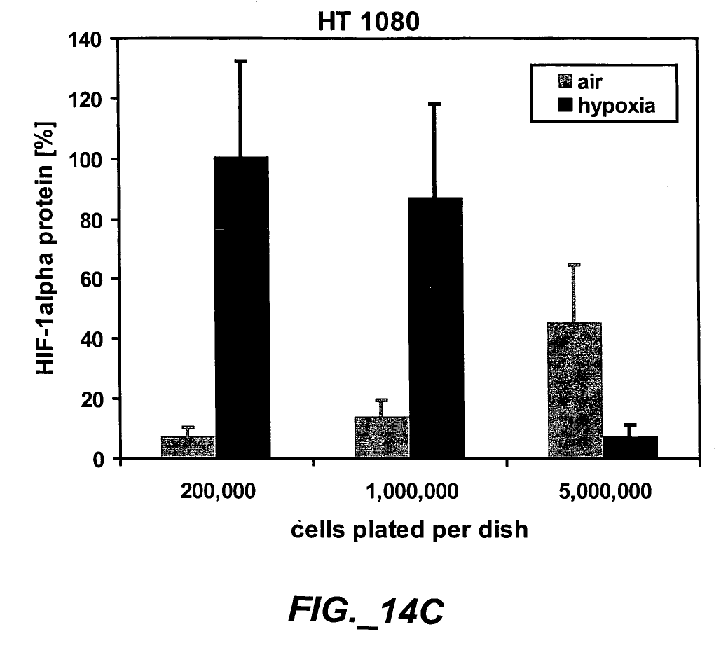

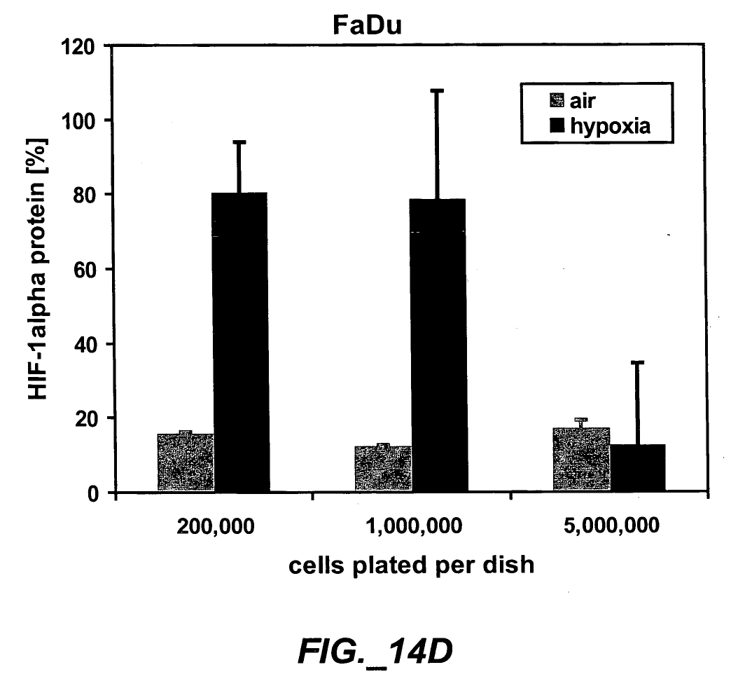

Plating of cells at high density (5×10⁶ per 80-mm dish) led to a strong increase in MN/CA IX protein levels compared to standard density (10⁶ per dish) under aerobic conditions in HT 1080 but not in FaDu cells (FIG. 14A-B). In HT 1080, the hypoxic MN/CA9 response was not affected by cell density. In FaDu high density caused a loss of MN/CA IX protein after hypoxia compared to standard density (FIG. 14A-B). Although whole-cell lysates were prepared from adherent monolayer cells, the high density caused significant detachment of cells in this cell line and loss of viability may have been preceded by disintegration of the membrane protein MN/CA IX.

To compare effects of cell density on MN/CA IX protein and on HIF-1α, a key transcription factor subunit regulating CA9 expression, HIF-1α protein was quantified in nuclear extracts from cells treated identically (FIG. 14C-D). HIF-1α protein also accumulated under aerobic high-density conditions (5×10⁶ per dish) in HT 1080 cells. In both HT 1080 and FaDu, a complete loss of the hypoxic HIF-1α response was observed at high cell density.

### Example 7

### MN/CA IX Flow Cytometry of Mixed Cell Suspensions and Comparison with Radiosensitivity

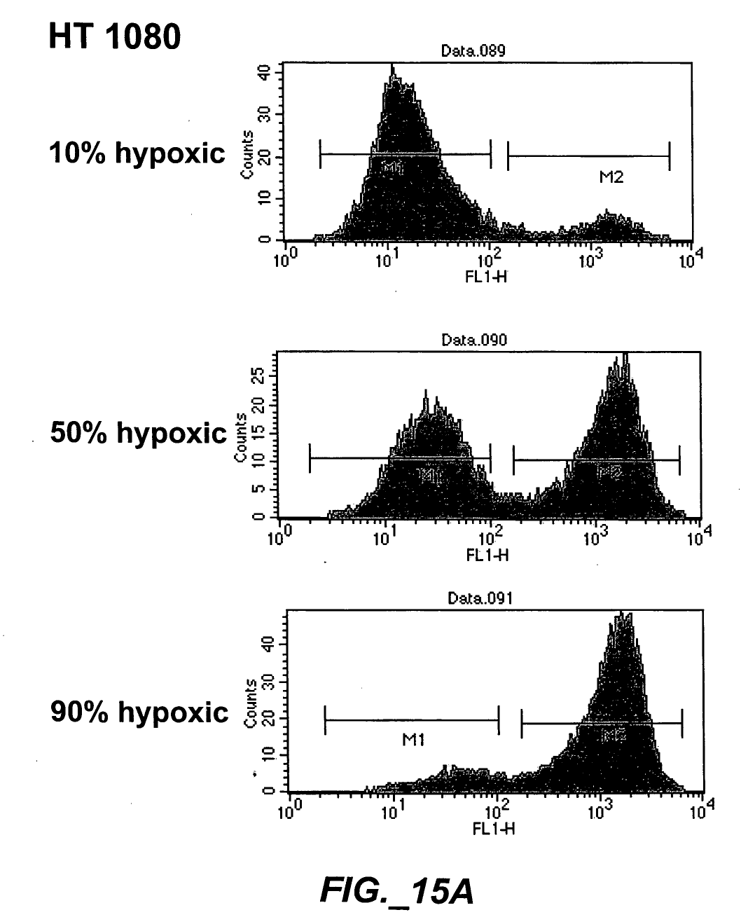

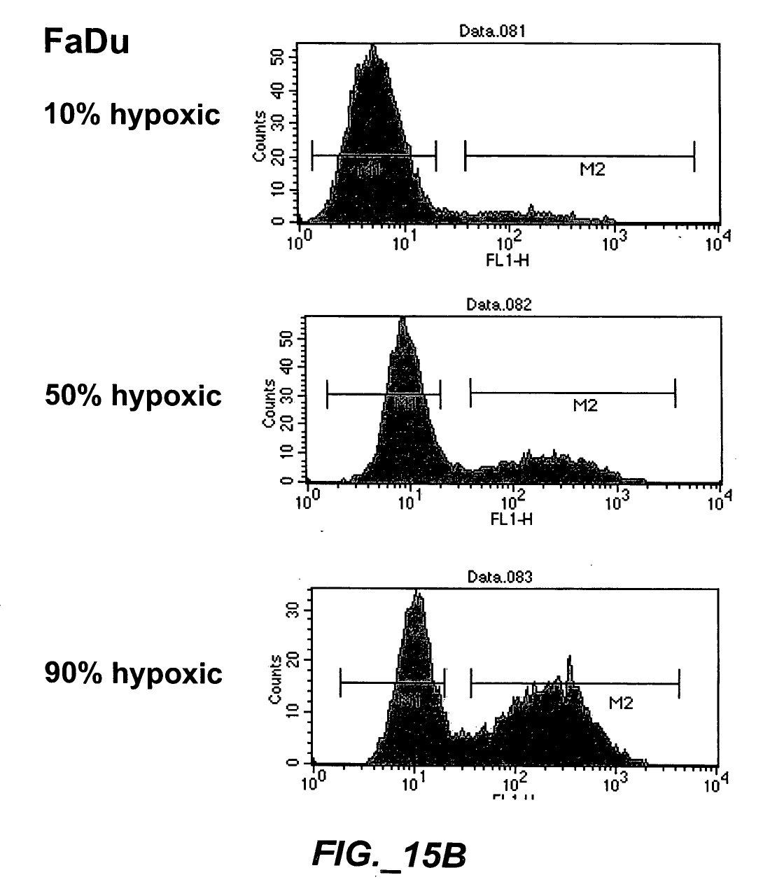

An initial protocol for MN/CA IX flow cytometry has been described by Olive et al. [Olive et al., Cancer Res., 61: 8924-8929 (2001)]. After testing various modifications, among them different modes of single-cell suspension preparation, different concentrations of the M75 monoclonal antibody against MN/CA IX and different secondary antibodies at various concentrations, the described protocol was found to give the largest spectrum between MN/CA IX-negative and -positive cell populations and lowest unspecific staining with flow cytometry. The FITC-anti-MN/CA IX fluorescence differed between positive and negative cells by a factor of 70 in HT 1080 and 30 in FaDu (FIG. 15A-B). In these experiments, the use of non-enzymatic preparation of cell suspensions was important for high FITC-anti-MN/CA IX fluorescence. For instance, in HT 1080 cells the above factor was reduced from 70 to about 25 with mild trypsin treatment. In FaDu, a factor of less than 3 was observed with mild trypsin treatment.

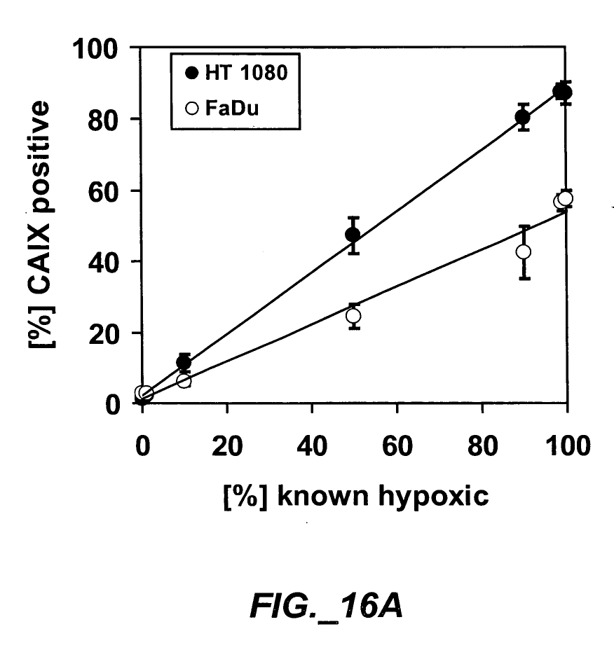

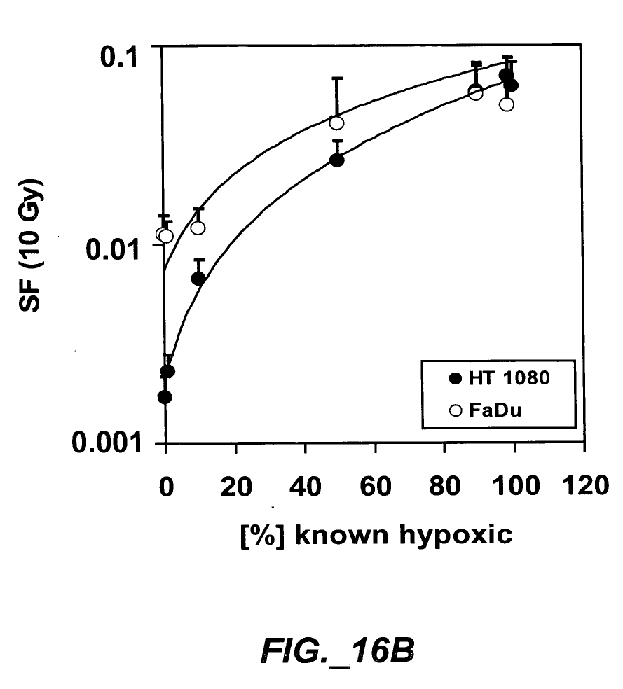

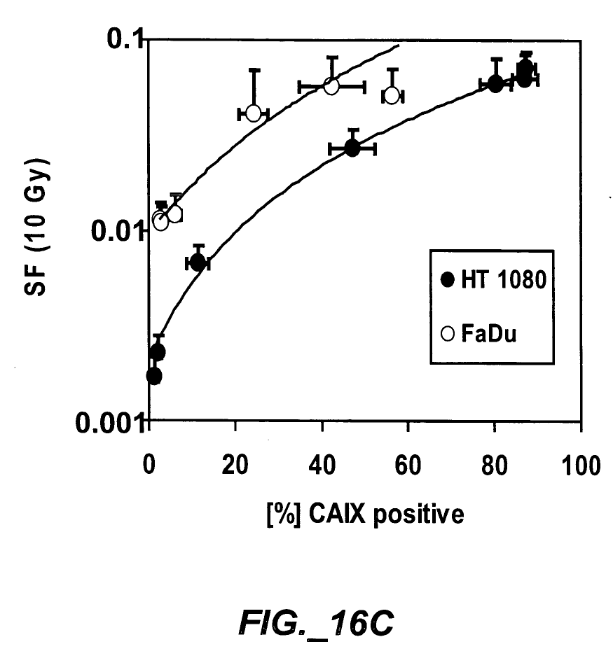

Using the final protocol, HT 1080 cells kept under aerobic conditions and cells treated with hypoxia (24 h, 0.1% O₂) could accurately be discriminated by MN/CA IX flow cytometry (FIG. 15A), and the measured percentages of MN/CA IX-positive cells closely reflected the known percentages of hypoxic cells (FIG. 16A). In FaDu, the percentage of MN/CA IX-positive cells was always lower than the known percentage of hypoxic cells (FIG. 15B), although there was still a linear correlation between the two parameters (FIG. 16A).

Plating of cell mixtures previously irradiated with a single dose of 10 Gy under aerobic or hypoxic conditions for clonogenic survival resulted in survival curves showing increased radiation resistance with increasing percentage of hypoxic cells (FIG. 16B).

When plotting the measured percentages of MN/CA IX-positive cells (from FIG. 16A) against the surviving fractions (from FIG. 16B), with each data point representing a known percentage of hypoxic cells, a better association of percentage of MN/CA IX-positive cells with radiation resistance was seen in HT 1080 than in FaDu (FIG. 16C). This was mainly a result of the more accurate detection of the percentage of hypoxic cells by MN/CA IX flow cytometry in HT 1080 (FIG. 16A).

Discussion

In the first part of the present study, the MN/CA IX protein levels were systematically analyzed after different schedules of in-vitro hypoxia and other manipulations of culture conditions in two cell lines, HT 1080 human fibrosarcoma and FaDu human pharyngeal carcinoma. The goal was to aid in the interpretation of the significance of MN/CA IX-positive cells in tumor sections and thereby define the role of MN/CA IX as a marker of tumor hypoxia, both for clinical and radiobiological applications. HT 1080 and FaDu have been previously used to characterize the patterns of accumulation of hypoxia-inducible factor-1α (HIF-1α) [Vordermark et al., Int. J. Radiat. Oncol. Biol. Phys., 58: 1242-1250 (2004); Vordermark et al., Cancer Lett., (in press)], a transcription factor subunit involved in the regulation of CA9 expression and also a potential hypoxia marker [reviewed in Vordermark and Brown, Strahlenther. Onkol., 179: 8018-8011 (2003)]. Therefore, the present results can be used to compare the properties of the two markers.

In both cell lines, a typical pattern of MN/CA IX protein accumulation after >6 h of hypoxia was observed (FIG. 10) with stability of the protein over 96 h of reoxygenation (FIG. 10-11). A concentration of 5% O₂ led to similar MN/CA IX protein levels as more severe hypoxia in HT 1080 cells, but to only half-maximal levels in FaDu (FIG. 10). This finding is the main explanation for the correlation of MN/CA IX protein levels with the radiation resistance of cells at the respective oxygen concentration in FaDu but not in HT 1080 (FIG. 12). The association of oxygen concentration and marker expression seen in MN/CA IX was nearly identical to that previously observed for HIF-1α [Vordermark et al, Int. J. Radiat. Oncol. Biol. Phys., 58: 1242-1250 (2004)], suggesting a tight regulation of CA9 by HIF-1. HIF-1α protein, however, showed a strong response after 1 h of hypoxia in a similar experimental setting [id.], and detectable HIF-1α protein has been reported by others within 2 min of hypoxia [Jewell et al., FASEB J., 15: 1312-1314 (2001)]. Detection of MN/CA IX in tumor sections, in contrast, must be regarded as an indicator of current or previous chronic hypoxia, as subsequent reoxygenation of up to 96 h, and possibly more, does not lead to degradation of the marker protein.

Previous studies in single cell lines have suggested a requirement of chronic hypoxia conditions for MN/CA IX overexpression. In an analysis of the transcriptional response to hypoxia by real-time polymerase-chain reaction (RT-PCR) in D247-MG human glioma cells, CA9 showed the strongest induction of all genes identified after 12 h of hypoxia [Lal et al., J Natl Cancer Inst., 93: 1337-1343 (2001)]. In U87 human glioma cells, upregulation of CA9 mRNA was 4-fold after 6 h and 13-fold after 12 h of hypoxia [Ivanov et al., Am. J. Pathol., 158: 905-919 (2001)]. An upregulation of CA9 mRNA and corresponding increase in MN/CA IX protein after 16 h at 0.1% O₂ was described for several cell lines, among them A549 and HeLa [Wykoff et al., Cancer Res, 60: 7075-7083 (2000)]. Although shorter treatments were not tested, graded hypoxia experiments in A549 showed an increase of MN/CA IX protein between 2.5% and 0.1% O₂ (16 h). Others have shown stability of CA9 mRNA over 8 h and of MN/CA IX protein over 72 h after reoxygenation in A549 [Turner et al., Br J Cancer, 86: 1276-1282 (2002)] and in HeLa cells the half-life of MN/CA IX protein has been estimated to be 38 h [Rafajova et al., Int. J. Oncol., 24: 995-1004 (2004)]. To the inventors' knowledge, these data are the first indicating that after 6 h of hypoxia, irrespective of its severity, no effect on MN/CA IX is seen at the protein level. Corresponding to the findings for HIF-1α, the fact that MN/CA IX protein was maximal after hypoxic treatment at 5% O₂ in HT 1080 cells, a condition hardly affecting radiosensitivity [Steel, Basic Clinical Radiobiology, 2^{nd} ed. London: Arnold (1997)], questions the use of MN/CA IX as a marker of radiobiologically relevant tumor hypoxia at least in the HT 1080 cell line.

Manipulation of glucose and serum availability, not of pH, all simulating in-vitro conditions of the tumor microenvironment, had dramatic effects on the hypoxic response of MN/CA IX protein (FIG. 13). Especially glucose concentration, even if only moderately reduced, led to a drastic decrease in MN/CA IX levels after 24 h of hypoxia at 0.1% O₂ (FIG. 17A-B). These findings for MN/CA IX are paralleled by similar results for HIF-1α in the same cell lines [Vordermark et al., Cancer Lett. (in press)]. Although interactions of different tumor microenvironment parameters were expected due to the role of carbonic anhydrases in the maintenance of pH homeostasis in and around tumor cells [Ivanov et al., Am. J. Pathol., 158: 905-919 (2001)], the reason for this finding is not clear and contrary findings have recently been reported. In HeLa cells, both hypoxia-induced transcription and MN/CA IX protein level were increased when glucose concentration was reduced [Rafajova et al., Int. J. Oncol., 24: 995-1004 (2004)]. This apparent discrepancy in the observed effects of glucose concentration of MN/CA IX induction may be explained by noting that the “low glucose” condition of Rafajova et al. (1.0 mg/ml) was equivalent to the “high glucose” condition (5.5 mM) of the experiments described here.

Finally, cell density also impacted on both aerobic and hypoxic MN/CA IX levels. In HT 1080, high cell density resulted in increased MN/CA IX protein under aerobic conditions with no effect in hypoxia (FIG. 14). In FaDu, the effect of high density in air was minimal, but the hypoxic response of CA9 was reduced, the latter associated with beginning detachment of cells. The induction of MN/CA IX in dense cultures has been characterized in detail by Kaluz and co-workers. They found that this induction requires the separate but interdependent pathways of phosphatidylinositol 3′-kinase (PI3K) and minimal HIF-1α levels [Kaluz et al., Cancer Res., 62: 4469-4477 (2002)] and that binding of transcription factors to both hypoxia-responsive elements (HREs) and an SP1/SP3 protected region (PR1) is required for cell-density dependent MN/CA IX induction [Kaluzova et al., Biotechniques, 36: 228-234 (2004)]. This group suggested that so-called “pericellular hypoxia” too mild for HIF-1α stabilization occurred in dense cultures. In fact, treatment of dense HeLa cultures with normobaric hyperoxia (50% O₂) abolished MN/CA IX expression [Chrastina et al., Neoplasma, 50: 251-256 (2003)].

The pattern of MN/CA IX protein under the different in-vitro conditions may explain some of the observations made in clinical series evaluating patient tumor material. Firstly, in the majority of reports, MN/CA IX immunostaining is described in regions compatible with chronic hypoxia. The Oxford group has found MN/CA IX positive areas to start at mean or median distances from the nearest blood vessel of 80 or 90 μm in head-and-neck squamous cell carcinoma [Beasley et al., Cancer Res., 61: 5262-5267 (2001)], non-small cell lung cancer [Swinson et al., J. Clin. Oncol., 21: 473-482 (2003)] and bladder cancer [Turner et al., Br J Cancer, 86: 1276-1282 (2002)]. They calculated that this distance equaled O₂ concentrations of approximately 1%.

Several groups have compared the labeling pattern of MN/CA IX with that of the injectable hypoxia marker pimonidazole which is thought to form intracellular adducts under chronic hypoxia conditions below about 1.5% O₂ [Gross et al., Int. J. Cancer, 61: 567-573 (1995)]. In cervix cancer sections, while reporting a good correlation between the two markers, Olive et al. found that MN/CA IX-positive regions extended beyond pimonidazole-positive areas in almost all cases [Olive et al., Cancer Res., 61: 8924-8929 (2001)], suggesting that MN/CA IX induction occurs at higher O₂ concentrations than 1.5%. Also in cervix cancer, Airley et al. noted distinctly similar staining patterns for MN/CA IX and pimonidazole, although the calculated correlation of the expression of the two markers was only of borderline significance [Airley et al., Int. J. Cancer, 104: 85-91 (2003)]. In head and neck cancer, Kaanders et al. found that MN/CA IX staining was more prominent in the regions 25-50 μm and 50-100 μm from the nearest blood vessel and pimonidazole staining was more intense in areas >100 μm from the vessel [Kaanders et al., Cancer Res., 62: 7066-7074 (2002)]. Considering the requirement of nutrients for a hypoxic MN/CA IX response shown in the present in-vitro study, one is tempted to speculate that such requirement may be the cause of decreased MN/CA IX staining in severely hypoxic but also nutrient-deprived tumor regions described by Kaanders et al. In the same study, a weak correlation of the two markers was seen, but only pimonidazole, not MN/CA IX, was prognostic for outcome. This again may have a possible explanation in the lack of MN/CA IX in some severely hypoxic and treatment-resistant tumor cells.

The present results for MN/CA IX are strikingly similar to previous results from this laboratory for HIF-1α obtained in the same cell lines [Vordermark et al, Int. J. Radiat. Oncol. Biol. Phys., 58: 1242-1250 (2004); Vordermark et al., Cancer Lett. (in press)], with the exception of the delayed time course of MN/CA IX induction. In patient material, only two groups have investigated the coexpression of the two markers. In studies of serial sections of nasopharyngeal carcinoma, 58% of cells showed typical nuclear staining for HIF-1α, and 57% had positive membrane staining for MN/CA IX, although without convincing overlap [Hui et al., Clin. Cancer Res., 8: 2595-2604 (2002)]. In non-small cell lung cancer 74% of MN/CA IX-positive cases were classified as having high HIF-1α expression, although the actual colocalization was not specified [Giatromanolaki et al., Cancer Res., 61: 7992-7998 (2001)].

The requirement of long-term hypoxia for MN/CA IX induction could well explain a lack of association between MN/CA IX staining in sections and measurement of tumor oxygenation with the Eppendorf probe, which is thought to measure both chronic and acute or intermittent forms of hypoxia. In a first study of advanced carcinoma of the cervix, patients treated with radiotherapy alone, however, the extent of MN/CA IX staining staining was siginificantly associated with all oxygenation parameters (HP2.5, HP5, HP10, median pO₂) and with disease-free and metastasis-free survival [Loncaster et al., Cancer Res., 61: 6394-6399 (2001)]. In a similar cohort of cervix cancer patients, a second group found neither a correlation of MN/CA IX staining with oxygenation nor an association of MN/CA IX with outcome [Hedley et al., Clin. Cancer Res., 9: 5666-5674 (2003)]. The authors discussed technical differences and intratumor heterogeneity as possible reasons for this discrepancy and suggested that factors additional to hypoxia might influence MN/CA IX levels. The latter assumption is well supported by the present in-vitro data.

Despite the concerns regarding the influence of non-hypoxic factors and the MN/CA IX accumulation already under mild hypoxia raised by this and previous studies, MN/CA IX still appears to be one of the most promising endogenous hypoxia markers due to its high stability. The fact that it only labels chronically hypoxic tumor cells may adversely affect its role in predicting treatment response in patients because acutely hypoxic cells remain undetected. However, this property is beneficial for studies of the radiation resistance of chronically hypoxic cells. The question whether chronically hypoxic cells in tumors are equally radiation resistant as acutely hypoxic cells or possibly similarly radiosensitive as normoxic cells, due to diminished DNA repair capacities, is an issue of debate [Vordermark et al., Radiat Res., 159: 94-101 (2003); Denekamp et al., Acta Oncol., 38: 903-918 (1999)]. Isolation of live chronically hypoxic cells from irradiated tumors on the basis of MN/CA IX status provides an elegant approach to determine the relative radiosensitivity of such cells. In contrast to previous studies from the inventor's laboratory, with xenografts from HT 1080 cells transfected with GFP under the control of a hypoxia-responsive promoter [Vordermark et al., Radiat. Res., 159: 94-101 (2003)], this method could be applied to experimental tumors from non-transfected cells and clinical tumors. Olive et al. have shown that sorting of live tumor cells by fluorescence-activated cell sorting (FACS) based on anti-MN/CA IX fluorescence is possible [Olive et al., Cancer Res., 61: 8924-8929 (2001)]. The 10% of cells from SiHa cervical carcinoma xenografts with the highest anti-MN/CA IX fluorescence were more radiation resistant than the 10% with the lowest fluorescence. Cells with high anti-MN/CA IX fluorescence had higher percentages of pimonidazole positivity.

In the second part of this study, a modified MN/CA IX flow cytometry protocol was developed, starting from the published procedure of Olive et al. [id.]. In addition to minor variations of the choice of secondary antibody and antibody incubation details, the observation was made that non-enzymatic preparation of single-cell suspensions from monolayer cell cultures was beneficial in preservation of the membrane protein MN/CA IX. This modification appears to have led to an improved width of fluorescence spectrum between MN/CA IX-positive and -negative cells. The Olive et al. group achieved a factor of about 20 in in-vitro experiments (P. Olive, personal communication) compared to a factor of about 70 (HT 1080) and 30 (FaDu) in the present study. Although this comparison is dependent on cell line used and experimental design, in the present experiments avoiding a trypsin treatment greatly improved separability of MN/CA IX-positive and -negative cells.

The final protocol was tested on mixed cell suspensions with known percentages of hypoxic cells prepared immediately after 24 h of hypoxia (0.1% O₂). In both cell lines, a linear correlation between percentage of MN/CA IX-positive cells and known percentage of hypoxic cells was observed (FIG. 16A). In FaDu, a fixed portion of hypoxic cells remained MN/CA IX-negative, whereas in HT 1080 the percentage of MN/CA IX-positive cells provided an accurate estimate of the percentage of hypoxic cells. Although the Western blots showed maximal MN/CA IX levels after 24 h at 0.1% in FaDu, this does not exclude a loss of the marker protein in some of the cells. The dramatic loss of hypoxic protein induction at high cell density in FaDu (FIG. 14B) may indicate that even at standard density, as used in the flow cytometry experiments, loss of MN/CA IX protein may be observable at the single-cell level.

Finally, it was shown that the known percentage of hypoxic cells in mixtures had a similar association with radiosensitivity (FIG. 16B) as did the percentage of MN/CA IX-positive cells (FIG. 16C) in these samples. Although the use of mixed cell suspensions is a simplified model of the oxygenation of cells expected within a tumor—excluding intermediate oxygen concentrations and any effects of non-hypoxic stimuli—these data document that the flow cytometry protocol described is capable of distinguishing hypoxic, MN/CA IX-positive, radioresistant HT 1080 and FaDu cells from better oxygenated, MN/CA IX-negative, radiosensitive cells. This is a prerequisite for the application of this protocol to sort cells by MN/CA IX expression from experimental tumors grown from these cells.

In conclusion, it has been shown through a systematic evaluation of MN/CA IX protein levels in HT 1080 and FaDu cells that MN/CA IX is a marker of chronic hypoxia, whether current or previous, but not acute hypoxia, that is stable during at least 96 h of reoxygenation, but influenced by nutrient availability and cell density. It is chronic hypoxia that is considered relevant for determining whether cells in a preneoplastic/neoplastic tissue are radioresistant. A modified protocol has been provided for MN/CA IX flow cytometry of non-enzymatically prepared cell suspensions that can be used to sort live chronically hypoxic cells from tumors by FACS.

### ATCC Deposits

The materials listed below were deposited with the American Type Culture Collection (ATCC) now at 10810 University Blvd., Manassus, Va. 20110-2209 (USA). The deposits were made under the provisions of the Budapest Treaty on the International Recognition of Deposited Microorganisms for the Purposes of Patent Procedure and Regulations thereunder (Budapest Treaty). Maintenance of a viable culture is assured for thirty years from the date of deposit. The hybridomas and plasmids will be made available by the ATCC under the terms of the Budapest Treaty, and subject to an agreement between the Applicants and the ATCC which assures unrestricted availability of the deposited hybridomas and plasmids to the public upon the granting of patent from the instant application. Availability of the deposited strain is not to be construed as a license to practice the invention in contravention of the rights granted under the authority of any Government in accordance with its patent laws.

| | Deposit Date | ATCC # |
|---|---|---|
| Hybridoma | | |
| VU-M75 | Sep. 17, 1992 | HB 11128 |
| MN 12.2.2 | Jun. 9, 1994 | HB 11647 |
| Plasmid | | |
| A4a | Jun. 6, 1995 | 97199 |
| XE1 | Jun. 6, 1995 | 97200 |
| XE3 | Jun. 6, 1995 | 97198 |

The description of the foregoing embodiments of the invention have been presented for purposes of illustration and description. They are not intended to be exhaustive or to limit the invention to the precise form disclosed, and obviously many modifications and variations are possible in light of the above teachings. The embodiments were chosen and described in order to explain the principles of the invention and its practical application to enable thereby others skilled in the art to utilize the invention in various embodiments and with various modifications as are suited to the particular use contemplated.

All references cited herein are hereby incorporated by reference.

1. A method for determining the degree of hypoxia in a tissue in a subject vertebrate, comprising:
(a) isolating a sample from said tissue from said vertebrate; and
(b) immunologically detecting and quantifying the level of MN/CA IX protein/polypeptide in said tissue;
wherein the level of said MN/CA IX protein/polypeptide found in said tissue in step (b), relative to the level of MN/CA IX protein/polypeptide found at 0.1% O₂ in comparable cells isolated from an organism of the same taxonomic classification as the subject vertebrate, indicates the degree of hypoxia in said tissue.

2. The method of claim 1, wherein said tissue is a preneoplastic/neoplastic tissue.

3. A method to predict the degree of radioresistance of an affected tissue in a subject vertebrate with a preneoplastic/neoplastic disease, wherein MN/CA IX protein/polypeptide levels in said preneoplastic/neoplastic tissue can be used to indicate radiobiologically relevant tumor hypoxia in said tissue, comprising:
(a) performing an in vitro test of comparable preneoplastic/neoplastic cells, correlating the MN/CA IX protein/polypeptide levels in said cells with degrees of cellular radioresistance, wherein said cells are isolated from an organism of the same taxonomic classification as the subject vertebrate;
(b) isolating a sample from the affected tissue in said subject vertebrate;
(c) immunologically detecting and quantitating the MN/CA IX protein/polypeptide level in said vertebrate sample; and
(d) predicting the degree of radioresistance of the subject vertebrate tissue by comparing the MN/CA IX protein/polypeptide level found in step (c) with the MN/CA IX protein/polypeptide levels of step (a), and extrapolating therefrom a predicted degree of radioresistance of the subject vertebrate tissue.

4. The method of claim 3, wherein said comparable preneoplastic/neoplastic cells are isolated from the subject vertebrate.

5. The method of claim 3, wherein said method is used as an aid in the selection of treatment for said preneoplastic/neoplastic disease afflicting said vertebrate.

6. The method of claim 5, wherein the predicted degree of radioresistance of the affected vertebrate tissue is high, and the decision is made not to use radiation therapy, or wherein the predicted degree of radioresistance of the affected vertebrate tissue is low, and the decision is made to use radiation therapy.

7. The method of claim 3, wherein said preneoplastic/neoplastic disease afflicting said subject vertebrate is selected from the group consisting of preneoplastic/neoplastic diseases of head and neck, mammary, urinary tract, kidney, bladder, ovarian, uterine, cervical, endometrial, vaginal, vulvar, prostate, liver, lung, skin, thyroid, pancreatic, testicular, brain, gastrointestinal, colon, colorectal and mesodermal tissues.

8. The method of claim 3 wherein said preneoplastic/neoplastic disease is head and neck preneoplastic/neoplastic disease.

9. The method of claim 3 wherein said subject vertebrate is a human patient.

10. The method of claim 9 wherein said neoplastic disease is a tumor, and said sample is taken from said tumor and/or from a metastatic lesion derived from said tumor.

11. The method of claim 3, wherein said sample is a formalin-fixed, paraffin-embedded tissue sample.

12. The method of claim 3, wherein said correlating step (a), and said detecting and quantitating step (c), comprise the use of an assay selected from the group consisting of Western blots, enzyme-inked immunosorbent assays, radioimmunoassays, competition immunoassays, dual antibody sandwich assays, immunohistochemical staining assays, agglutination assays, and fluorescent immunoassays.

13. The method according to claim 3, wherein said correlating step (a), and said detecting and quantitating step (c), comprise the use of the M75 monoclonal antibody secreted by the hybridoma VU-M75 which has Accession No. ATCC HB 11128.

14. The method of claim 3, wherein said detecting and quantitating step (c) comprises the use of immunohistochemical staining with MN/CA IX-specific antibody to detect the levels of MN/CA IX protein in the sample.

15. The method according to claim 14, wherein said detecting and quantitating step (c) further comprises determining the percentage of MN/CA IX immunoreactive cells and/or the intensity of immunostaining of immunoreactive cells.

16. The method of claim 3, wherein said correlating step (a) comprises determining the oxygen enhancement ratio (OER) or modified oxygen enhancement ratio (OER′) in said comparable preneoplastic/neoplastic cells.

17. The method of claim 9, wherein said preneoplastic/neoplastic disease is head and neck cancer, and wherein said comparable preneoplastic/neoplastic cells are FaDu human pharyngeal carcinoma cells.

18. The method of claim 9, wherein said preneoplastic/neoplastic disease is head and neck cancer, and wherein said comparable preneoplastic/neoplastic cells are isolated from the subject human.

19. A method to detect cells that are both hypoxic and metabolically active in a prenoplastic/neoplastic tissue derived from a subject vertebrate, comprising:
(a) isolating a sample from the affected tissue in said subject vertebrate; and
(b) immunologically detecting and quantitating the MN/CA IX protein/polypeptide level in said vertebrate sample;
wherein if detectable MN/CA IX protein/polypeptide is found in step (b), concluding that the cells in the vertebrate sample are both hypoxic and metabolically active; or if no detectable MN/CA IX protein/polypeptide is found in step (b), concluding that the cells in the vertebrate sample are not hypoxic and/or not metabolically active.

20. The method of claim 19, wherein if no detectable MN/CA IX protein/polypeptide is found in step (b), further comprising screening said vertebrate sample for a second hypoxic marker;
wherein if said second hypoxic marker indicates that the cells are hypoxic, further concluding that said cells in the vertebrate sample are hypoxic but not metabolically active.

21. The method of claim 19, wherein said method is used as an aid in the selection of treatment for said preneoplastic/neoplastic disease afflicting said vertebrate.
